(19) United States
(12) Patent Application Publication
(10) Pub. No.: US 2022/0235292 A1
(43) Pub. Date: Jul. 28, 2022

(54) SYSTEMS AND METHODS FOR STABILIZING EMULSIONS

(71) Applicant: Dropworks, Inc., Boulder, CO (US)

(72) Inventors: Matthew Ryan Dunn, Louisville, CO (US); Andrew Carl Larsen, Superior, CO (US); Christopher Michael Perkins, Boulder, CO (US)

(21) Appl. No.: 17/615,782

(22) PCT Filed: Jun. 3, 2020
(86) PCT No.: PCT/US20/70116

§ 371 Date: Dec. 1, 2021

Related U.S. Application Data
(60) Provisional application No. 62/856,660, filed on Jun. 3, 2019.

(51) Int. Cl. C11D 1/94; C09K 23/08

(52) U.S. Cl. CPC C11D 1/94; C09K 23/08

(57) ABSTRACT

Provided herein are methods and compositions related to forming cross-links between surfactant molecules at an interface between dispersed phase partitions and a continuous phase in an emulsion, and uses of such cross-linked emulsions.

### REFERENCE TO RELATED APPLICATIONS

This application claims the benefit of the priority date of U.S. provisional application 62/856,660, filed Jun. 3, 2019, the contents of which are incorporated herein in their entirety.

### BACKGROUND

Emulsion systems made up of partitions of dispersed phase in a continuous phase are useful in numerous applications. Potential drawbacks of emulsions systems include movement of molecules from on partition to another, coalescence, and the like.

### SUMMARY

In one aspect, provided herein are compositions.

In certain embodiments, provided herein is a composition comprising a plurality of first surfactant molecules comprising a tail portion and a head portion, wherein the first surfactant molecules comprise an average of 2-10 of a first linkage moiety per surfactant molecule wherein the first linkage moieties are configured to either (i) link with each other under suitable conditions, (ii) to link with a second linkage moiety under suitable conditions, wherein the second linkage moiety is attached to a second surfactant molecule comprising a tail portion and a head portion, and wherein the second linkage moiety is different from the first linkage moiety, or (iii) to link with an intermediate linkage moiety under suitable conditions, or a combination thereof. The first linkage moieties are attached to the tail portion of the first surfactant molecules. Alternatively, the first linkage moieties can be attached to the head portion of the first surfactant molecules. The second linkage moieties can be attached to the head portion of the second surfactant molecules. Alternatively, the second linkage moieties can be attached to the tail portion of the second surfactant molecules. In certain embodiments, the linkage moieties are configured to form one or more covalent bonds under suitable conditions. In certain embodiments, the linkage moieties are configured to form one or more noncovalent bonds under suitable conditions. In certain embodiments the composition further comprises a plurality of the second surfactant molecules, wherein the second surfactant molecules comprise an average of 2-10 of the second linkage moiety per surfactant molecule. In certain embodiments, the first and second linkage moieties are oppositely charged. In certain embodiments, the composition further comprises a continuous phase containing the first surfactant molecules. In certain embodiments, the composition further comprises a continuous phase containing the second surfactant molecules. In certain embodiments, the composition further comprises a dispersed phase; in some of these embodiments, the dispersed phase does not contain the first surfactant molecule; in some of these embodiments, the dispersed phase does not contain the second surfactant molecule. In certain embodiments where an intermediate linkage moiety is used, the dispersed phase contains the intermediate linkage moiety. In certain embodiments, the composition further comprises a dispersed phase containing the first surfactant molecules. In certain of these embodiments, the composition further comprises a continuous phase. In certain embodiments the composition further comprises a dispersed phase containing the second surfactant molecules. In certain of these embodiments, the composition further comprises a continuous phase, for example a continuous phase that does not contain the first surfactant molecule and/or a continuous phase does not contain the second surfactant molecule. In certain embodiments, the continuous phase contains the intermediate linkage moiety. In certain embodiments, the surfactant moieties comprise the first linkage moiety comprising biotin and the intermediate linkage moieties comprising one or more biotin-binding moieties, such as streptavidin or a streptavidin derivative. In certain embodiments, the surfactant molecules form micelles in the continuous phase, such as a continuous phase comprising an oil. In certain embodiments, the oil is a hydrocarbon or a silicon oil. In certain embodiments, the oil comprises a fluorinated oil. In certain embodiments, the surfactant is a fluorosurfactant.

In certain embodiments provided herein is composition comprising an emulsion comprising partitions of a dispersed phase in a continuous phase, wherein the partitions of the dispersed phase comprise a plurality of surfactant molecules comprising a tail portion and a head portion that are situated at the interface of the partitions with the continuous phase to form a layer of surfactant molecules, and wherein the plurality of surfactant molecules are cross-linked to each other to form a cross-linked network of surfactant molecules. In certain embodiments, the degree of cross-linking of the cross-linked network of surfactant molecules is 20-100%. In certain embodiments, the cross-linking is via cross links having an average length of 5-500% of the length of the tail portion of the surfactant. In certain embodiments, the cross-linking is via cross links having an average length of 5-500% of the length of the head portion of the surfactant. In certain embodiments, the surfactant molecules are cross-linked to each other at the tail portions of the surfactant molecules. In certain embodiments, the surfactant molecules are cross-linked to each other at the head portions of the surfactant molecules. In certain embodiments, a first portion of the surfactant molecules comprise a first linkage moiety that forms part of the cross-links, and wherein the average number of first linkage moieties per surfactant molecule is 2-10. In certain embodiments, a second portion of the surfactant molecules comprise a second linkage moiety, different from the first linkage moiety, that forms part of the cross-links, and wherein the average number of second linkage moieties per surfactant molecule is 2-10. In certain embodiments, the surfactant molecules are cross-linked via cross-links, and where the average length of the cross-links is 1-100 nm. In certain embodiments the dispersed phase comprises an aqueous phase and the continuous phase comprises an oil, in certain cases the oil comprises a fluorinated oil. In certain embodiments, the surfactant comprises a fluorosurfactant. In certain embodiments, the surfactant molecules are cross-linked by noncovalent bonds. In certain embodiments, the surfactant molecules are cross-linked by covalent bonds. In certain embodiments, the surfactant molecules comprise biotin moieties that are cross-linked by biotin-binding moieties. In certain embodiments the biotin-binding moieties comprise streptavidin or a streptavidin derivative. In certain embodiments, the surfactant molecules comprise an average of 2-10 biotin moieties per surfactant molecule. In certain embodiments, the cross-linked network of surfactant molecules increases the stability of the partitions in the emulsion, compared to the same emulsion without a cross-linked surfactant network, as measured by a decrease in dye diffusion of at least 20% in a dye diffusion test comprising, such as in one of the dye diffusion assays described herein, e.g., diffusion of rodamine CG, or diffusion of resorufin, or diffusion of fluorescein. In certain embodiments, cross-linked network of surfactant molecules increases the stability of the partitions in the emulsion, compared to the same emulsion without a cross-linked surfactant network, as measured by a PCR test, such as a PCR test as described herein, of at least 20%. In certain embodiments, the cross-linked network of surfactant molecules increases the stability of the partitions in the emulsion, compared to the same emulsion without a cross-linked surfactant network, as measured by a coalescence assay, such as one of the coalescence assays described herein, of at least 20%.

In certain embodiments provided herein is a composition comprising a continuous phase wherein the continuous phase comprises a plurality of surfactant molecules that comprise linkage moieties attached to the surfactant molecules, wherein the moieties form cross-links with each other under suitable conditions. In certain embodiments the continuous phase is to be used in the preparation of an emulsion. In certain embodiments the moieties do not substantially interact to cross-link in the continuous phase. In certain embodiments the moieties form non-covalent cross-links under suitable conditions. In certain embodiments the moieties form covalent cross-links under suitable conditions. In certain embodiments the continuous phase comprises an oil. In certain embodiments the oil is a fluorinated oil. In certain embodiments the fluorinated oil is (3-ethoxy-1,1,1,2,3,4,4,5,5,6,6,6-dodecafluoro-2-trifluoromethyle-hexane), methyl nonafluorobutyl ether, methyl nonafluoroisobutyl ether, ethyl nonafluoroisobutyl ether, ethyl nonofluorobutul ether, (pentane, 1,1,1,2,2,3,4,5,5,5-decafluoro-3-methoxy-4-(trifluoromethyl-)), isopropyl alcohol, (1,2-trans-dicholorethylene), (butane,1,1,1,2,2,3,3,4,4-nonafluoro-4-methoxy-), (1,1,1,2,2,4,5,5,5-nonafluoro-4-(trifluoromethyl)-3-pentanone), (furan,2,3,3,4,4-pentafluorotetrahydro-5-methoxy-2,5-bis[1,2,2,2-tetrafluoro-1-(trifluoromethyl)ethyl]-), perfluoro compounds comprising between 5 and 18 carbon atoms, polychlorotrifluoroethylene, (2,2,2-trifluoroethanol), Novec 8200™, Novec 71DE™, Novec 7100™, Novec 7200DL™, Novec 7300DL™, Novec 71 IPA™, Novec 72FL™, Novec 7500™, Novec 71DA™, Novec 7100DL™, Novec 7000™, Novec 7200™, Novec 7300™, Novec 72DA™, Novec 72DE™, Novec 649™, Novec 73DE™, Novec 7700™, Novec 612™, FC-40™, FC-43™, FC-70™, FC-72™, FC-770 ™, FC-3283™, FC-3284™, PF-5056™, PF-5058™, Halocarbon 0.8™, Halocarbon 1.8™, Halocarbon 4.2™, Halocarbon 6.3™, Halocarbon 27™, Halocarbon 56™, Halocarbon 95™, Halocarbon 200™, Halocarbon 400™, Halocarbon 700™, Halocarbon 1000N™, Uniflor 4622R™, Uniflor 8172™, Uniflor 8472CP™, Uniflor 8512S™, Uniflor 8731™, Uniflor 8917™, Uniflor 8951™, TRIFLUNOX 3005™, TRIFLUNOX 3007™, TRIFLUNOX 3015™, TRIFLUNOX 3032™, TRIFLUNOX 3068™, TRIFLUNOX 3150™, TRIFLUNOX 3220™, or TRIFLUNOX 3460™ or a combination thereof. In certain embodiments the fluorinated oil is (3-ethoxy-1,1,1,2,3,4,4,5,5,6,6,6-dodecafluoro-2-trifluoromethyle-hexane); (furan,2,3,3,4,4-pentafluorotetrahydro-5-methoxy-2,5-bis[1,2,2,2-tetrafluoro-1-(trifluoromethyl)ethyl]-); perfluoro compounds comprising between 5 and 18 carbon atoms; or a combination thereof. In certain embodiments the surfactants comprise fluorosurfactants. In certain embodiments the fluorosurfactant comprises fluorosurfactants having head and tail moieties linked by ether, amide, or carbamide bonds; fluorosurfactants having a polyethylene moiety linked to a fluorocarbon moiety through a carbamide, ether, or amide bond or a combination thereof. In certain embodiments the fluorosurfactant comprises a polyethylene moiety linked to a fluorocarbon moiety with a carbamide, amide, or ether bond. In certain embodiments the cross-links formed under suitable conditions comprise direct cross-links. In certain embodiments the cross-links formed under suitable conditions comprise indirect cross-links. In certain embodiments the linkage moieties comprise biotin. In certain embodiments each surfactant molecule comprises an average of at least 2 linkage moieties. In certain embodiments each surfactant molecule comprises an average of at least 4 linkage moieties. In certain embodiments the linkage moieties of the surfactant molecules are such that when they react under suitable conditions, the resulting cross-link between surfactant molecules is 1-100 nm. In certain embodiments the cross-link that forms under suitable conditions between linkage moieties has a bond strength of between 20 and 200 kJ/mole. In certain embodiments the concentration of surfactant molecules is 0.5% to 2% w/v.

In certain embodiments provided herein is a composition comprising a dispersed phase for use in formation of an emulsion of partitions of the dispersed phase in a continuous phase wherein the partition interface with the continuous phase in the emulsion comprises a plurality of surfactant molecules comprising cross-linking moieties, wherein the dispersed phase comprises one or more components that initiate and/or promote a cross-linking process between the cross-linking moieties under suitable conditions. In certain embodiments the dispersed phase is not in contact with a continuous phase. In certain embodiments the dispersed phase comprises an aqueous phase. In certain embodiments the dispersed phase further comprises one or more additional components that initiate and/or promote a non-cross-linking process under suitable conditions. In certain embodiments the non-cross-linking process comprises a chemical reaction. In certain embodiments the components comprise nucleic acids and components for conducting polymerase chain reaction. In certain embodiments the cross-linking process comprises a covalent interaction. In certain embodiments the cross-linking process comprises a non-covalent process. In certain embodiments the cross-linking process comprises linking linkage moieties of the surfactant molecules through one or more intermediate linker moieties, and wherein the dispersed phase further comprises a plurality of the intermediate linker moieties. In certain embodiments the intermediate linker moiety comprises a biotin-binding moiety. In certain embodiments the biotin-binding moiety comprises avidin, streptavidin, a streptavidin derivative, or a combination thereof. In certain embodiments the concentration of the one or more components is 10 nmolar to 10 mmolar.

In certain embodiments provided herein is an emulsion composition comprising a plurality of partitions of a dispersed phase in a continuous phase, wherein (i) interfaces between the dispersed phase partitions and the continuous phase comprises a plurality of surfactant molecules; (ii) the surfactant molecules of the interfaces are cross-linked by one or more linkage moieties between surfactant molecules; (iii) at least a portion of the partitions contain first components that, under suitable conditions, undergo a process to produce second components; and (iv) the continuous phase comprises reporter moieties that interact with the second components to produce a signal indicating the presence and/or abundance of the second components. In certain embodiments the first and second components are the same or nearly the same. In certain embodiments the first component is a nucleic acid of interest and the second component is a product of amplification of the nucleic acid. In certain embodiments the linkage moieties have one or more properties that promote entrance of the reporter molecule into the partitions.

In certain embodiments provided herein is a composition comprising a plurality of first surfactant molecules comprising a tail portion and a head portion, wherein the first surfactant molecule comprises a first linkage moiety attached to the first surfactant molecule, wherein the first linkage moiety is configured to participate in formation of cross-links between the first surfactant molecule and a second surfactant comprising a second linkage moiety under suitable conditions. In certain embodiments the first and second linkage moieties have the same structure. In certain embodiments the first surfactant molecule comprises a plurality of linkage moieties. In certain embodiments the first surfactant molecule is attached to an average of 2-10 linkage moieties. In certain embodiments the first linkage moiety is attached to the tail portion of the surfactant molecule. In certain embodiments the first linkage moiety is attached to the head portion of the surfactant molecule. In certain embodiments the length of the first linkage moiety is 5-500% of the length of the head portion of the surfactant molecule. In certain embodiments the first surfactant molecule is a nonionic, anionic, cationic, or zwitterionic surfactant. In certain embodiments the first surfactant molecule is a fluorosurfactant. In certain embodiments the linkage moiety is covalently attached to the surfactant molecule. In certain embodiments the linkage moiety is configured to bind to an intermediate moiety under suitable conditions and not to another linkage moiety. In certain embodiments the linkage moiety comprises biotin. In certain embodiments the intermediate moiety comprises a biotin-binding moiety. In certain embodiments the plurality of first surfactant molecules is contained in a continuous phase.

In certain embodiments provided herein is a kit comprising(i) a container comprising a surfactant for use in forming an emulsion, wherein the surfactant molecules comprise a plurality of linkage moieties for cross-linking to other surfactant molecules; (ii) packaging that contains the container of (i).

In one aspect, provide herein are methods.

In certain embodiments, provided herein is method of conducting a process in an emulsion of partitions of a dispersed phase in a continuous phase, comprising (i) providing the emulsion of partitions of dispersed phase in continuous phase, wherein the partitions of the dispersed phase comprise a plurality of surfactant molecules comprising a tail portion and a head portion that are situated at an interface of the partitions with the continuous phase to form a layer of surfactant molecules, and wherein the plurality of surfactant molecules are cross-linked to each other to form a cross-linked network of surfactant molecules; and (ii) performing the process on the partitions. In certain embodiments, the degree of cross-linking of the cross-linked network of surfactant molecules is 20-100%. In certain embodiments, the cross-linking is via cross links having an average length of 5-500% of the length of the tail portion of the surfactant, or of the head portion of the surfactant. In certain embodiments, the cross-linking is via cross links having an average length of 5-500% of the length of the head portion of the surfactant, or of the head portion of the surfactant. In certain embodiments, the surfactant molecules are cross-linked to each other at the tail portions of the surfactant molecules. In certain embodiments, the surfactant molecules are cross-linked to each other at the head portions of the surfactant molecules. In certain embodiments, a first portion of the surfactant molecules comprise a first linkage moiety that forms part of the cross-links, and wherein the average number of first linkage moieties per surfactant molecule is 2-10. In certain embodiments, a second portion of the surfactant molecules comprise a second linkage moiety, different from the first linkage moiety, that forms part of the cross-links, and wherein the average number of second linkage moieties per surfactant molecule is 2-10. In certain embodiments, the surfactant molecules are cross-linked via cross-links, and where the average length of the cross-links is 1-100 nm. In certain embodiments, the dispersed phase is an aqueous phase and the continuous phase is a oil. In certain embodiments, the oil is a fluorinated oil. In certain embodiments, the surfactant comprises a fluorosurfactant. In certain embodiments, the surfactant molecules are cross-linked by noncovalent bonds. In certain embodiments, the surfactant molecules are cross-linked by covalent bonds. In certain embodiments, the surfactant molecules are cross-linked by biotin-biotin binding moiety interactions. In certain embodiments, the biotin-binding moieties comprise streptavidin or a streptavidin derivative. In certain embodiments, the surfactant molecules comprise an average of 15, 1-10, 2-10 biotin moieties per surfactant molecule. In certain embodiments, the method further comprises forming the cross-linked network of surfactant molecules in the partitions. In certain embodiments, the cross-linked network of surfactant molecules has been formed by contacting continuous phase comprising a plurality of surfactant molecules that comprise at least one linkage moiety with a dispersed phase under conditions wherein the dispersed phase forms a plurality of partitions in the continuous phase, and providing conditions during and/or after the formation of the partitions that initiate and/or promote formation of cross-links comprising the linkage moieties, wherein the linkage moieties form cross-links from one surfactant molecule to at least one other surfactant molecule, to form a cross-linked network of surfactant molecules. In certain embodiments, dispersed phase comprises one or more components that initiate and/or promote formation of cross-links comprising the linkage moieties when in contact with the linkage moieties. In certain embodiments, the partitions are exposed to an external stimulus that initiates and/or promotes formation of cross-links comprising the linkage moieties. In certain embodiments, the process comprises chemical analysis; protein or strain engineering; nucleic acid, protein, or cell-based assays; sorting; separations; or chemical and/or biochemical synthesis; or a combination thereof. In certain embodiments, the process comprises a nucleic acid assay. In certain embodiments, the process comprises polymerase chain reaction (PCR). In certain embodiments, the cross-linked network of surfactant molecules increases the stability of the partitions in the emulsion, compared to the same emulsion without a cross-linked surfactant network, as measured by a decrease in dye diffusion of at least 20% in a dye diffusion test, such as one of the dye diffusion tests described herein, e.g., diffusion of rodamine CG, or diffusion of resorufin, or diffusion of fluorescein. In certain embodiments, the cross-linked network of surfactant molecules increases the stability of the partitions in the emulsion, compared to the same emulsion without a cross-linked surfactant network, as measured by a PCR test, such as a PCR test as described herein, of at least 20%.

In certain embodiments provided herein is a method for producing an emulsion of partitions of dispersed phase in continuous phase, wherein the partitions of the dispersed phase comprise a plurality of surfactant molecules comprising a tail portion and a head portion that are situated at an interface of the partitions with the continuous phase to form a layer of surfactant molecules, comprising (i) contacting continuous phase with a dispersed phase, wherein either (a) the continuous phase comprises a plurality of surfactant molecules that comprise at least one linkage moiety, or (b) the dispersed phase comprises a plurality of surfactant molecules that comprise at least one linkage moiety, or (c) both (a) and (b) under conditions wherein the dispersed phase forms a plurality of partitions in the continuous phase; and (ii) providing conditions during and/or after the formation of the partitions that initiate and/or promote formation of cross-links between surfactant molecules comprising the linkage moieties, to form a cross-linked network of surfactant molecules. In certain embodiments, the continuous phase comprises surfactant molecules comprising linkage moieties and the dispersed phase does not. In certain embodiments, the linkage moieties and other components of the cross-links, if present, form cross-links that have a length that is 5-500% of the length of the tail portion of the surfactant, or 5-500% of the length of the head portion of the surfactant. In certain embodiments, the surfactant molecules comprise an average of 2-10 linkage moieties per surfactant molecule. In certain embodiments, the conditions and/or number of linkage moieties are such that the degree of completion of the cross-linked network of surfactant molecules is 20-100%. In certain embodiments, the linkage moieties are attached to the tail portions of the surfactant molecules and the cross-links form between the tail portions of the surfactant molecules. In certain embodiments, the linkage moieties are attached to the head portions of the surfactant molecules and the cross-links form between the head portions of the surfactant molecules. In certain embodiments, dispersed phase comprises one or more components that initiate and/or promote formation of cross-links comprising the linkage moieties when in contact with the linkage moieties. In certain embodiments, the one or more components comprise one or more intermediate linkage moieties that form one or more bonds with the surfactant linkage moieties. In certain embodiments, the surfactant linkage moieties comprise biotin and the intermediate linkage moieties comprise biotin-binding moieties. In certain embodiments, the biotin-binding moieties comprise streptavidin and/or streptavidin derivatives. In certain embodiments, the partitions are exposed to an external stimulus that initiates and/or promotes formation of cross-links comprising the linkage moieties. In certain embodiments, the external stimulus comprises light. In certain embodiments, the continuous phase comprise an oil and the dispersed phase comprises an aqueous phase. In certain embodiments, the oil comprises a fluorinated oil. In certain embodiments, the surfactant comprises a fluorosurfactant. In certain embodiments, the cross-linked network of surfactant molecules increases the stability of the partitions in the emulsion, compared to the same emulsion without a cross-linked surfactant network, as measured by a decrease in dye diffusion of at least 20% in a dye diffusion test such as in one of the dye diffusion assays described herein, e.g., diffusion of rodamine CG, or diffusion of resorufin, or diffusion of fluorescein. In certain embodiments, the cross-linked network of surfactant molecules increases the stability of the partitions in the emulsion, compared to the same emulsion without a cross-linked surfactant network, as measured by a PCR test, such as a PCR test as described herein, of at least 20%.

In certain embodiments provided herein is a method of preparing an emulsion comprising a plurality of partitions of dispersed aqueous phase in an oil continuous phase, wherein the partitions further comprise a cross-linked network of surfactant molecules at the surface of the partitions, comprising preparing an aqueous phase to be dispersed, preparing an oil phase comprising modified surfactant, wherein the modified surfactant comprises a tail portion and a head portion and further comprises linkage moieties; and mixing the aqueous phase and the oil phase to form an emulsion of a plurality of partitions of the aqueous phase in the oil, wherein the modified surfactant molecules form cross-links with each other to form a cross-linked network of surfactant molecules at the interface of the partitions with the continuous phase. In certain embodiments, the mixing is done in bulk by vortexing, pipetting, syringing, shaking. In certain embodiments, the mixing is by a microfluidic droplet forming device. In certain embodiments, the mixing is by a microfluidic T-junction, flow focusing junction, reverse-y junction, millipede junction or a combination thereof. In certain embodiments, a system for producing the emulsion is embedded within a larger instrument. In certain embodiments, the instrument is an instrument containing a sample delivery module, a droplet generator module, a thermal cycler module, a detection module, a waste management module, or a combination thereof. In certain embodiments, the larger instrument has microfluidic devices, tubing, containers or vats embedded. In certain embodiments, the instrument comprises associated software that controls the instrument including but not limited to the performance of the instrument as a whole or the microfluidic device.

In certain embodiments provided herein is method for preventing coalescence of partitions in an emulsion comprising partitions of dispersed phase in a continuous phase, comprising forming a cross-linked network of surfactant molecules at interfaces between the partitions and the continuous phase. In certain embodiments, the partitions have an average diameter of greater than 1 um diameter. In certain embodiments, the emulsion is at a temperature greater than 60° C.

In certain embodiments, provided herein is a method for transporting, thermal cycling, incubating, sorting, or analyzing an emulsion comprising partitions of dispersed phase in a continuous phase in a microfluidic device, wherein the droplets comprising a network of cross-linked surfactant molecules at the interface of the partitions and the continuous phase.

In certain embodiments, provided herein is a method of performing PCR in partitions of an emulsion comprising partitions of aqueous phase in a continuous phase, wherein the partitions comprise cross-linker, polymerase, nucleotides, template DNA, primers, and probes/or DNA binding dyes, and the continuous phase comprises surfactant molecules comprising one or more linkage moieties.

In certain embodiments, provided herein is a method comprising (i) forming an emulsion comprising a plurality of partitions comprising dispersed phase, in a continuous phase, wherein the partitions comprise a surfactant layer comprising a plurality of surfactant molecules at an interface with the continuous phase; (ii) cross-linking surfactant molecules at the interface to form a cross-linked surfactant network; (iii) performing a process on the partitions; and (iv) treating the cross-linked surfactant network to decrease a degree of cross-linking. In certain embodiments, the method further comprises breaking open a plurality of the partitions to release dispersed phase in the partitions. In certain embodiments, the surfactants comprise fluorosurfactants and the continuous phase comprises a fluorinated oil. In certain embodiments, the process comprises a polymerase chain reaction.

In certain embodiments provided herein is a method of performing an emulsion flow process comprising (i) providing a continuous phase comprising (a) a continuous phase, and(b) surfactant molecules with linkage moieties; (ii) providing a dispersed phase, separate from the continuous phase, that does not comprise surfactant molecules with linkage moieties, and that comprises one or more components that initiate and/or promote formation of cross-links between surfactant molecules, wherein the cross-links comprise the linkage moieties; (iii) flowing the continuous phase and the dispersed phase into a partition generator that generates an emulsion of a plurality of partitions comprising the dispersed phase in the continuous phase, and (iv) during and/or after partition formation, forming cross-links between surfactant molecules that comprise the linkage moiety to form a cross-linked network of surfactant molecules at the interface of the partitions with continuous phase; and (v) flowing the emulsion through a process system that performs one or more operations on the partitions of the emulsion.

In certain embodiments provided herein is a method of modifying a first surfactant molecule comprising a tail portion and a head portion, comprising attaching a plurality of first linkage moieties to the first surfactant molecule, wherein the first linkage moiety is configured to participate in formation of cross-links between the first surfactant molecule and a second surfactant molecule comprising a second linkage moiety under suitable conditions. In certain embodiments the method comprises attaching an average of 2-10 of the first linkage moiety to the first surfactant molecule. In certain embodiments the method comprises attaching the plurality of first linkage moieties to the tail portion of the surfactant molecule. In certain embodiments the method comprises attaching the plurality of first linkage moieties to the head portion of the surfactant molecule. In certain embodiments the length of the first linkage moiety is 5-500% of the length of the head portion of the surfactant molecule; in certain embodiments the length of the first linkage moiety is 5-500% of the length of the tail portion of the surfactant molecule. In certain embodiments the first surfactant molecule is a nonionic, anionic, cationic, or zwitterionic surfactant In certain embodiments, the first surfactant molecule is a fluorosurfactant. In certain embodiments the method comprises covalently attaching the linkage moiety to the first surfactant molecule. In certain embodiments the linkage moiety is configured to bind to an intermediate moiety under suitable conditions and not to another linkage moiety. In certain embodiments the linkage moiety comprises biotin. In certain embodiments the intermediate moiety comprises a biotin-binding moiety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows dispersed phase partitioning into continuous phase to form an emulsion.

FIG. 2 shows a schematic representation of the coalescence of two or more partitions.

FIG. 3 shows a schematic representation of two modes of molecular transit across a partition interface.

FIG. 4 shows a schematic representation of molecular transit between separate partitions in an emulsion.

FIG. 5 shows an embodiment of surfactant cross-linking.

FIG. 6 shows different degrees of cross linking; left, full cross-linking; right, partial cross-linking.

FIG. 7 shows surfactants embedded in the partition interface may vary in physical geometry.

FIG. 8 shows an embodiment of control of molecular transit though modification of the chemical properties of the partition interface.

FIG. 9 shows an embodiment of reduction of molecular transit by inhibition of reverse micelle formation.

FIG. 10 shows an embodiment of controlled molecular transit through size-based limitations of surfactant network porosity.

FIG. 11 shows effects of varying linker size.

FIG. 12 shows effect of cross-linking agent number on extent of cross-linking.

FIG. 13 shows head-to-head cross-linking and tail-to-tail cross-linking of surfactant moieties.

FIG. 14 shows direct cross-linking between linkage moieties on two different surfactant molecules.

FIG. 15 shows cross-linking of two surfactant moieties by ionic bond.

FIG. 16 shows indirect cross-linking between two surfactant moieties via one or more intermediate linkage moieties.

### DETAILED DESCRIPTION OF THE INVENTION

Disclosed herein are compositions and methods related to the stabilization of emulsions by promoting surfactant-surfactant interactions at the partition interface, herein referred to as cross-linking. Surfactant cross-linking imbues emulsions with increased stability by forming a molecular network, i.e., a surfactant network, at the partition interface. Such a network comprises surfactant molecules that interact in such a way as to tend to keep the surfactant molecules associated and/or to decrease movement of surfactant molecules; in some instances, the association is promoted by the use of linkage moieties attached to the surfactant molecules that interact, either directly or via an intermediate moiety to cross-link the surfactant molecules, where the interaction is covalent in some cases and noncovalent in other cases, and where the interaction keeps the cross-linked surfactant molecules associated. As used herein, “surfactant molecule” and “surfactant moiety,” used interchangeably, refer to a single surfactant entity; such an entity may be a single molecule or an assemblage of more than one molecule into a multimolecular complex. Surfactants in general have a hydrophilic head portion and a hydrophobic tail portions; cross-links between surfactant molecules may be between head groups, between tail groups, head-to-tail, or a combination thereof. In certain embodiments, the continuous phase comprises an oil, such as a fluorinated oil, the dispersed phase comprises an aqueous phase, and the surfactants comprise fluorosurfactants. The molecular (surfactant) network has the ability to alter the boundary properties of the droplets to help promote or restrict the movement of molecules into or out of the droplet. Disclosed herein are methods for preparing emulsions, preparing surfactants, as well as resulting emulsions bearing cross-linked surfactants, and other resulting compositions. Additionally, disclosed herein are methods using and uses of the described surfactants and emulsions bearing the cross-linked surfactants. Additionally disclosed is the utility of the surfactants and emulsions of the surfactants in a partition handling system.

Compositions and methods described herein can be used with any suitable emulsion system that utilizes a surfactant. In certain embodiments, the emulsion system is a water-in-oil emulsion system, where an aqueous dispersed phase is partitioned in an oil continuous phase, with surfactant used to stabilize the partition interface and surfactant molecules cross-linked, either partially or completely, as described herein. In certain embodiments, surfactant molecules comprise one or more linkage moieties, also referred to as linker moieties herein, that can either directly join together between surfactant molecules, e.g. in a covalent or non-covalent linkage between a linkage moiety on one surfactant and a linkage moiety on another surfactant, or that indirectly join together via one or more linkage molecules, i.e. intermediate linkage moieties, where the intermediate linkage (linkage) moiety, e.g., molecule joins with a linkage moiety on one surfactant molecule, e.g., in a covalent or non-covalent bond, and joins with a linkage moiety on at least one other surfactant molecule, e.g., in a covalent or non-covalent bond. Any suitable method for introducing suitable linkage moieties to the surfactant molecules may be used; in certain cases, the surfactant molecules already contain such moieties without modification, and in other cases surfactant molecules are modified to include one or more linkage moieties, e.g., by attaching linkage moieties covalently or noncovalently. Methods include modifying surfactant molecules to be used in an aqueous dispersed phase to comprise one or more linkage moieties, and providing conditions for cross-linking, e.g., when the surfactants are forming or have formed an interface between the partition and the continuous phase.

Any suitable method may be used to cross-link the surfactant molecules. Prior to formation of the emulsion, surfactant molecules configured to cross-link can be present in the continuous phase, in the dispersed phase, or a combination thereof. In certain cases, surfactant molecules comprising linkage moieties are present in a continuous phase, e.g., the modified surfactant molecules are present in a continuous phase, and one or more components that activate, promote, or facilitate a linkage process between linkage moieties on the surfactant molecules, such as one or more activating component to promote a linkage process between linker moieties on the surfactants, or such as a linker molecule, i.e., an intermediate linker moiety, is present in dispersed phase, such as an aqueous phase; the phases are brought together to form an emulsion of partitions of the dispersed phase, e.g., aqueous phase in the continuous phase, for example, in a partition generator. The surfactant forms a layer at the interface of the partitions and the continuous phase, and surfactant molecules are cross-linked by formation of bonds, e.g., directly between surfactant linkage moieties or indirectly via one or more intermediate linker moieties that form a cross-link between the linker moieties on the surfactant molecules and the linker molecules, i.e., intermediate linker moiety. In certain embodiments, surfactant molecules are modified to comprise one or more, for example, an average of 1-20, 1-15, 1-10, 2-20, 2-15, 2-10, 2-8, 2-6, 3-20, 3-15, 3-10, 3-8, 3-5, 4-20, 4-15, 4-10, 4-8, 5-20, 5-15, 5-10, 5-9, 6-20, 6-15, or 6-10 linkage moieties. The optimum number of linkage moieties per surfactant can be dependent on the type of cross-linking reaction and the use, in some cases, of a linker molecule, i.e., intermediate linker moiety. The attachment between the surfactant and the linkage moieties may be any suitable type of attachment, e.g., a covalent bond or a noncovalent bond. In certain embodiments, surfactant molecules are modified to comprise two different linkage moieties; in general, a first set of modified surfactant molecules is prepared with a first linkage moiety attached and a second set of modified surfactant molecules is prepared with a second linkage moiety attached. The first and second sets of surfactant molecules can be combined to produce a composition containing a plurality of both first and second modified surfactants. Such modified surfactant molecules may be used in, e.g., reactions where part of the first linkage moiety reacts with part of the second linkage moiety to produce a direct cross-link, or with intermediate linkage moiety to produce an indirect cross-link.

In certain embodiments, surfactant molecules are modified to comprise one or more, for example an average of 1-20, 1-15, 1-10, 2-20, 2-15, 2-10, 2-8, 2-6, 3-20, 3-15, 3-10, 3-8, 3-5, 4-20, 4-15, 4-10, 4-8, 5-20, 5-15, 5-10, 5-9, 6-20, 6-15, or 6-10, for example, an average of about 6, biotin moieties, and are cross-linked by addition of biotin-binding moiety, e.g., streptavidin or a streptavidin derivative, which binds to biotin moieties, thus acting as an intermediate linkage moiety. The intermediate linkage moiety, e.g., streptavidin, may be present in any suitable quantity prior to cross-linking; for example, the amount of cross-linker necessary to coat the entirety of the surface of an average partition may be calculated, and some percentage of that amount may be used, e.g., 1, 5, 10, 20, 50, 70, 100, 120, 150, 200, 300, 400, or 500%, or any range therebetween, in some cases adjusting for the likely number of partitions. In certain embodiments, the surfactant is a fluorosurfactant. In certain embodiments, the continuous phase comprises oil, for example, a fluorinated oil.

Compositions and methods disclosed herein may find beneficial applications in any suitable emulsion-based setting including but not limited to chemical analysis, protein and strain engineering, nucleic acid, protein, and cell-based assays, sorting, or separations, or chemical and/or biochemical synthesis, for example, decreasing or eliminating small molecule transfer between partitions in combinatorial drug synthesis, high throughput drug screening, analysis of products secreted by individual cells, directed evolution of desired enzymes, construction of synthetic cells, or any other suitable use of an emulsion system, in particular uses where movement of entities between partitions is not desirable. For convenience, compositions and methods may be described in relation to polymerase chain reaction digital PCR, but one of skill in the art will recognize that the same or similar compositions and methods may be used in any suitable emulsion system.

Droplet microfluidics, in which droplets act as individual compartments, has enabled a wide range of applications including but not limited to digital PCR, high throughput screening, strain and protein engineering, and cell, protein, and chemical analysis. Some examples include but are not limited to DNA/RNA amplification (Mazutis et al., A.D. Lab Chip, 2009, 9, 2665-2672; Mazutis et al., Anal. Chem., 2009, 81(12), 4813-4821), in vitro transcription/translation (Courtois et al., Chembiochem., 2008, 9(3), 439-446), enzymatic catalysis (Baret et al., Lab Chip, 2009, 9(13), 1850-1858), and cell-based assays (Clausell-Tormos et al., Chem. Biol., 2008, 15(8), 427-437; Brouzes et al., Proc. Natl. Acad. Sci. USA, 2009, 106(34), 14195-14200). The tiny size of the microdroplets, ranging from 1 picoliter to 1 milliliter in volume, and their relative orthogonality and isolation from other droplets in their respective populations facilitates screening and other processes at extremely high throughputs (>10⁴ samples per second) and vastly reduced reagent consumption.

Emulsions are suspensions of a first liquid phase (sometimes referred to as the “dispersed phase”) in a second liquid phase (sometimes referred to as the “continuous phase”) substantially immiscible with the first liquid phase. In some embodiments, emulsions are generated by partitioning the dispersed phase into one or more continuous phases. Due to the higher affinity of molecules of each respective phase for other molecules in that phase than for molecules in the other phase, merging or coalescence of more than one dispersed phase partition into larger combined partitions in the absence of stabilizing agents is generally thermodynamically favored. Surfactants are commonly used as such stabilizing agents in the production of emulsions between two substantially immiscible fluids to prevent coalescence; in some embodiments, these two substantially immiscible fluids are aqueous phases and oils. In combination with microfluidic technologies, a plethora of applications have been developed for generating, modulating, altering, and transporting stable and uniformly sized water-in-oil (WO) and water-in-oil-in-water (WOW) as well as oil-in-water (OW) and oil-in-water-in-oil (OWO) partitions (sometimes known as “droplets”) with aims of expanding or enhancing emulsion-based applications. In many cases methods and compositions provided herein will be described in terms of WO emulsions but it will be appreciated that the same principles and techniques can be applicable to WOW, OW, and WO emulsions, as appropriate.

In embodiments in which continuous or dispersed phase comprises an oil, any suitable oil may be used, such as hydrocarbon oils, silicon oils, etc. In certain embodiments, a fluorinated oil is used, e.g., as continuous phase and/or for other fluid components as described further herein. Fluorinated oils may comprise (3-ethoxy-1,1,1,2,3,4,4,5,5,6,6,6-dodecafluoro-2-trifluoromethyle-hexane), methyl nonafluorobutyl ether, methyl nonafluoroisobutyl ether, ethyl nonafluoroisobutyl ether, ethyl nonofluorobutul ether, (pentane, 1,1,1,2,2,3,4,5,5,5-decafluoro-3-methoxy-4-(trifluoromethyl-)), isopropyl alcohol, (1,2-trans-dicholorethylene), (butane,1,1,1,2,2,3,3,4,4-nonafluoro-4-methoxy-), (1,1,1,2,2,4,5,5,5-nonafluoro-4-(trifluoromethyl)-3-pentanone), (furan,2,3,3,4,4-pentafluorotetrahydro-5-methoxy-2,5-bis[1,2,2,2-tetrafluoro-1-(trifluoromethyl)ethyl]-), perfluoro compounds comprising between 5 and 18 carbon atoms, polychlorotrifluoroethylene, (2,2,2-trifluoroethanol), Novec 8200™, Novec 71DE™, Novec 7100™, Novec 7200DL™, Novec 7300DL™, Novec 71 IPA™, Novec 72FL™, Novec 7500™, Novec 71DA™, Novec 7100DL™, Novec 7000™, Novec 7200™, Novec 7300™, Novec 72DA™, Novec 72DE™, Novec 649™, Novec 73DE™, Novec 7700™, Novec 612™, FC-40™, FC-43™, FC-70™, FC-72™, FC-770™, FC-3283™, FC-3284™, PF-5056™, PF-5058™, Halocarbon 0.8™, Halocarbon 1.8™, Halocarbon 4.2™, Halocarbon 6.3™, Halocarbon 27™, Halocarbon 56™, Halocarbon 95™, Halocarbon 200™, Halocarbon 400™, Halocarbon 700™, Halocarbon 1000N™, Uniflor 4622R™, Uniflor 8172™, Uniflor 8472CP™, Uniflor 8512S™, Uniflor 8731™, Uniflor 8917™, Uniflor 8951™, TRIFLUNOX 3005™, TRIFLUNOX 3007™, TRIFLUNOX 3015™, TRIFLUNOX 3032™, TRIFLUNOX 3068™, TRIFLUNOX 3150™, TRIFLUNOX 3220™, or TRIFLUNOX 3460™. In certain embodiments, the fluorinated oil comprises (3-ethoxy-1,1,1,2,3,4,4,5,5,6,6,6-dodecafluoro-2-trifluoromethyle-hexane), (furan,2,3,3,4,4-pentafluorotetrahydro-5-methoxy-2,5-bis[1,2,2,2-tetrafluoro-1-(trifluoromethyl)ethyl]-), and/or perfluoro compounds comprising between 5 and 18 carbon atoms and the fluorosurfactant comprises a polyethylene moiety linked to a fluorocarbon moiety with a carbamide, amide, or ether bond.

Surfactants appropriate for their respective oils are usually necessary for stable emulsion formation. Traditional surfactants are generally not suitable for stabilizing emulsions of aqueous phase in fluorinated oil due to low solubility of the non-polar end of the surfactant in the fluorinated oil. In addition, they are often toxic to biological molecules and cells resulting in loss or alteration of activity in the partitions as compared to standard systems.

Fluorinated surfactants suitable for stabilizing emulsions have been developed that reduce toxicity issues. However, while a dramatic increase in partition stability has been achieved by advances in fluorosurfactant chemistry, there are many applications where further improvement to the stability of the WO and WOW emulsions can enable the development of new applications. Some examples include the ability to work with larger partitions (e.g. equivalent spherical diameters that are >5 um, >10 um, >20 um, >30 um, >40 um, >50 um, >60 um, >80 um, >70 um, >100 um, >200 um, >500 um) in high shear force environments, at high temperatures, in the presence of electric fields, e.g., static or other potentials, and/or with specific aqueous formulations that would normally destabilize the partition interface. Additionally, there is a need to further modify the diffusion and/or adsorption of molecules between the two phases or at the interface.

Thus, in certain embodiments a surfactant is a fluorinated surfactant. In certain embodiments, fluorosurfactants comprise an oligoethylene glycol, TRIS, or polyethylene glycol moiety. In certain embodiments, fluorosurfactants comprise a fluorocarbon and/or chlorofluorocarbon moiety. In some embodiments, fluorosurfactants have head and tail moieties linked by ether, amide, or carbamide bonds. In a certain embodiments, fluorosurfactants have a polyethylene glycol moiety linked to a fluorocarbon moiety through a carbamide, ether, or amide bond. Fluorinated surfactants include but are not limited to Picosurf-1, Ran FS-008, FC-4430, FC-4432, FC-4434. in certain cases, the fluorosurfactant can comprise a polyethylene moiety linked to a fluorocarbon moiety with a carbamide, amide, or ether bond. In certain embodiments in which biotin is used as a linkage moiety, an exemplary fluorosurfactant, including biotin, is FS-Biotin from Ran Biotechnologies. See, e.g., US Patent Application Publication No. 20180112036. Fluorosurfactant can have a concentration between 0.01% w/v to 5% w/v in the fluorinated oil. In certain embodiments, fluorosurfactant concentration ranges from 0.5% to 2% w/v, such as 0.5-1.5%. In general herein, surfactant concentrations are expressed as a percentage of surfactant in continuous phase, e.g., the percentage of surfactant in the continuous phase as it is flowed into a partitioner to produce partitions of dispersed phase.

In embodiments in which a fluorinated oil and/or fluorosurfactant is used, it is advantageous to have surfaces of the system that is used to process the emulsion that are fluorinated, e.g., at least 80, 90, 95, or 99% of the surfaces that come in contact with the emulsion during processing, in order to reduce, e.g., potential holdup of partitions at the surface. Thus, in certain embodiments, the surface of the passages, e.g., conduits in the system comprises a fluoropolymer, at least one continuous phase comprises a fluorinated oil, and the dispersed phase has a lower affinity for a fluoropolymer surface than the fluorinated oil. Here and elsewhere herein, a fluoropolymer may be any suitable fluoropolymer, such as polytetrafluoromethylene (PTFE), chlorotrifluoroethylene (CTFE), polyvinylidene difluoride (PVDF), perfluoroalkoxy polymer (PFA), fluorinated ethylene-propylene (FEP), or a combination thereof. In some embodiments, the surface of the conduits comprises a hydrophilic material, at least one continuous phase is hydrophilic, and at the dispersed phase is hydrophobic. In a further embodiment, the dispersed phase is an oil.

In certain embodiments, the fluorinated oil comprises (3-ethoxy-1,1,1,2,3,4,4,5,5,6,6,6-dodecafluoro-2-trifluoromethyle-hexane), (furan,2,3,3,4,4-pentafluorotetrahydro-5-methoxy-2,5-bis[1,2,2,2-tetrafluoro-1-(trifluoromethyl)ethyl]-), and/or perfluoro compounds comprising between 5 and 18 carbon atoms and the fluorosurfactant comprises a polyethylene moiety linked to a fluorocarbon moiety with a carbamide, amide, or ether bond.

In an example, digital assays are performed by considering an ensemble of partitions of a dispersed phase. In such assays, an analyte of interest is distributed among the partitions upon generation and measuring a property of each partition allows for the determination of whether that partition comprises a minimum amount of the analyte of interest. In some embodiments/examples, by correlating the numbers of partitions that measured as containing and/or not containing, and/or containing a certain amount of, the analyte of interest to an underlying statistical distribution of the analyte among partitions, a property of the original analyte may be ascertained. In an embodiment, the property is a concentration, such as a molar concentration or a volumetric concentration, or any other suitable expression of concentration.

Polymerase chain reaction (PCR) is a method used to amplify a desired nucleic acid in vitro. By carefully measuring the rate of amplification, the initial concentration of nucleic acid may be quantified using a standard reference. However, many factors complicate the deduction of initial quantity including but not limited to (1) poor PCR efficiency, (2) low concentration of starting template, (3) reaction termination, and (4) byproduct formation. Furthermore, differences in amplification efficiency between the reference material and target nucleic acid may skew the derivation of actual concentration.

For example, digital PCR (‘dPCR’) performed using microfluidically generated partitions improves upon the current PCR practices to quantify a DNA template by partitioning the sample into many smaller reactions. The dispersed phase is partitioned utilizing a microfluidic device resulting in a population of partitions of highly uniform size ranging in volume from milliliters to femtoliters. By partitioning the sample into enough partitions that there is at least one partition devoid of a template, performing the compartmentalized amplification in parallel, and then counting the number of partitions that contained and did not contain template, the concentration of target DNA may be calculated using Poisson statistics. Since there is no need for a reference for quantification, dPCR eliminates a prominent source of error in nucleic acid quantification. dPCR can further improve precision for rare target detection by increasing the initial volumetric concentration of a template within a given partition. For example, 1 template in a solution would see a 20,000-fold increase in concentration when the reaction is partitioned into 20,000 equally sized partitions. Such increases in template concentration may result in an increase in chemical reaction kinetics and amplification efficiency for those rare pieces of DNA. Rare target detection can also benefit from a change in the ratio of rare target to a complex background for the same reasons. As the template concentration is increased there is a relative decrease in the concentration of inhibitory molecules that can influence reactions, providing additional resistance to the impacts of those inhibitory molecules.

The accuracy of digital assays is reliant on, among other factors, the number of partitions the system is able to generate, the percentage of the sample emulsified, the stability of the resulting partitions as they are handled, incubated, and analyzed, and the retention of aqueous constituents and reporter molecules in the partition of interest. In some cases, an influx of reporter molecules is beneficial [Prodanovic et. al, 2011, Combinatorial Chemistry & High Throughput Screening]. For example, the selective transfer of DNA detection molecules (such as intercalation agents) from the oil phase to the aqueous may be desirable. This type of transfer can relatively eliminate a detection ceiling caused by having a limited amount of the reagent in the aqueous phase. Therefore, innovations that increase the stability and decrease coalescence of partitions as well as either reduce or selectively limit molecule diffusion between phases and/or partitions are encompassed by certain embodiments provided herein.

In general, compositions and methods provided herein can be used in emulsion systems where it is desired to decrease or eliminate movement of components from one partition to continuous phase and, usually, to another partition, and/or to modulate movement into or out of partitions of selected components. Without being bound by theory, it is thought that inter-partition movement of components can occur through coalescence, where two or more partitions join together into a larger partition, thus mingling the contents of each partition; through reverse micelle formation and movement; and through direct diffusion of components out of partitions into continuous phase, and, usually, back into other partitions. Alternatively or additionally, compositions and methods provided herein for cross-linking surfactants can make the surfactant interface between partitions and continuous phase more robust; thus, partitions are more resistant to, e.g., harsh reaction conditions, or to components within the partitions that would, in the absence of cross-linking, destabilize the partitions.

To stabilize emulsions against coalescence, surfactants are used to lower the interfacial tension and thus the Gibbs free energy, provide steric or electrostatic repulsion, increase film drainage time, or increase the surface elasticity. Emulsifiers are often amphiphilic molecules comprising groups soluble in each of the two phases. When present in a single solvent, either aqueous or oil, they form micellular structures. At the time of and for some period after partition formation and the generation of the partition interface, the micelles disperse and adsorb to an oil-water interface.

Depending of the specific chemistry of the surfactant, the surfactants may be more soluble in either the aqueous or oil phases. In some cases, the surfactants form a micelle in either phase and upon formation of the partition interface, the micelles dissociate, and the surfactant molecules embed themselves in the partition interface. Since the surfactants are amphiphilic and have at least two groups of opposed solubility, the tail and head groups associate with their respective solvents. This association with the interface results in improved partition stability. The respective solubility equilibrium reached between the micelle state and the interface state likely has a role in partition stability and interface integrity.

Coalescence may be exacerbated chemically by reducing the solubility of the surfactant in one of the two phases. In some examples, such reductions in solubility may be through pH adjustments or the additions of chemicals that interact with or alter the chemical composition of the surfactant, oil, or aqueous solution.

Transit of chemicals from the continuous phase to the dispersed phase or between partitions in the continuous phase may be driven by the dynamic equilibrium of the surfactant between the micelle and interface state.

Since many of the forces that promote partition instability are not easily mutable in most partition applications, a new method for improving partition stability would, e.g., increase the drainage time and stabilize the surfactant in the presence of high temperature, mechanical forces, for example shear forces, electric fields, and/or chemical reagents present in many biotechnological formulations.

### Definitions

As used herein the term “emulsion” includes a mixture of two immiscible fluids such that one of the two phases (dispersed phase) forms individual partitions contained within the second (continuous) phase. Common emulsions can be oil suspended in water, or aqueous phase, (OW) or water suspended in oil (WO). Other common emulsions can be multiple emulsions like water-in-oil-in-water (WOW) or oil-in-water-in-oil (OWO).

As used herein the term “creaming” includes a separation of an emulsion into two emulsions, one of which (the cream) is richer in the disperse phase than the other. Creaming may be a precursor to coalescence.

As used herein the term “sedimentation” includes the settling of partitions from an emulsion because of the density difference between the two phases. Sedimentation may be a precursor to coalescence.

As used herein the term “flocculation” includes the grouping together of partitions in an emulsion without a change in surface area. This term is exchangeable with aggregation.

As used herein the term “Ostwald ripening” includes the phenomena in which smaller partitions merge with larger partitions in order to reach a more thermodynamically stable state wherein the surface to area ratio is minimized.

As used herein the term “coalescence” includes the fusion of two or more partitions to form larger partitions.

As used herein the term “surfactant” includes a substance which tends to reduce the surface tension of a liquid in which it is dissolved thereby helping stabilize an emulsion. As used herein, “surfactant molecule” and “surfactant moiety” are generally synonymous, and can include a surfactant with more than one molecule in its structure.

As used herein the term “Gemini surfactants” includes dimeric surfactants that contain a spacer unit between two surfactant molecules.

As used herein the term “fluorinated” includes any group or substance which contains one or more fluorine atoms. Generally, the group or substance contains multiple fluorine atoms. For example, a fluorinated oil refers to any oil containing fluorine atoms, including but not limited to partially fluorinated hydrocarbons, perfluorocarbons, hydrofluoroethers and mixtures thereof.

As used herein the term “fluorosurfactant” includes any chemical surfactant with at least one fluorocarbon moiety.

As used herein a “cross-linking agent” includes a component of one of the fluid phases that has the ability to link two or more surfactant molecules resulting in the formation of a molecular network at the partition interface.

As used herein, “cross-linking” of surfactant molecules, for example, surfactant molecules at the interface of a partition of dispersed phase in a continuous phase, refers to formation of bonds between surfactant molecules. The bonds may be covalent or non-covalent, and generally will not form, or will not form to any substantial degree, between surfactant molecules without modification of the surfactant molecules. The modification can include addition of one or more linkage moieties to the surfactant molecules, which are then cross-linked either directly or indirectly, and/or exposure of the surfactant molecules to conditions suitable for forming cross-links between the surfactant molecules. In general, “cross-linking,” as used herein, refers to the use of linkage moieties attached to the fluorosurfactant molecules, rather than modification of the head or tail of the surfactant molecules, unless made clear otherwise by context.

As used herein the term “dispersed aqueous phase” includes a water-based solution that may comprise one or more analytes of interest. In some embodiments, dispersed aqueous phases comprise at least one chemical reagent and act as discrete reaction vessels for at least one chemical reaction. Systems comprising a dispersed aqueous phase may additionally comprise a detector that may measure a property of the at least one chemical reaction in at least one of the discrete reaction vessels. In some embodiments, the dispersed aqueous phase may comprise an analyte of interest and a property of the at least one chemical reaction may be correlated to a property of the analyte of interest. In some embodiments, the measurement is digital in nature, in the sense that the potential results of the measurement may be represented by a discrete set. In further embodiments, a value of at least one measurement above or below a threshold value determines whether at least one discrete reaction vessel contains the analyte of interest. In other embodiments, the measurement is analog in nature, in the sense that the potential results of the measurement are represented by a continuous range of values. In some embodiments, the aqueous phase may comprise buffers, salts, analytes, stabilizers, surfactants, dyes, or any combination thereof as described below.

As used herein the term “continuous phase” includes a liquid substantially immiscible with the dispersed phase and in which the partitions reside either before, during, or after dispersed phase partitioning. The continuous phase may refer to the immiscible phase used to partition the dispersed phase into partitions or a new immiscible phase the partitions were exchanged into after formation.

As used herein the term “stabilizers” include a broad class of molecules that may include preservatives, molecules that help to maintain or enhance the stability or activity of enzymes, molecules that help to inhibit or inactivate particular types of enzymes, for example, surfactants, metal ions, sugars, crowding agents, DNAse or RNAse inhibitors.

As used herein the term “stability” includes the maintenance of the partition interface resulting in a reduction in droplet breakage and/or coalescence and/or, modification of molecular diffusion through the partition interface, such as a decrease in reverse micelle formation, and a decrease or increase in molecular diffusion across the interface; whether diffusion is increased or decreased depends on the particular molecule and the nature of the partition interface.

As used herein, the term “kit” includes a collection of items intended for use together. The items in the kit may or may not be in operative connection with each other. A kit can comprise, e.g., reagents, buffers, enzymes, antibodies and other compositions specific for the purpose. A kit can also include instructions for use and software for data analysis and interpretation. A kit can further comprise samples that serve as normative standards. Typically, items in a kit are contained in primary containers, such as vials, tubes, bottles, boxes or bags. Separate items can be contained in their own, separate containers or in the same container. Items in a kit, or primary containers of a kit, can be assembled into a secondary container, for example a box or a bag, optionally adapted for commercial sale, e.g., for shelving, or for transport by a common carrier, such as mail or delivery service.

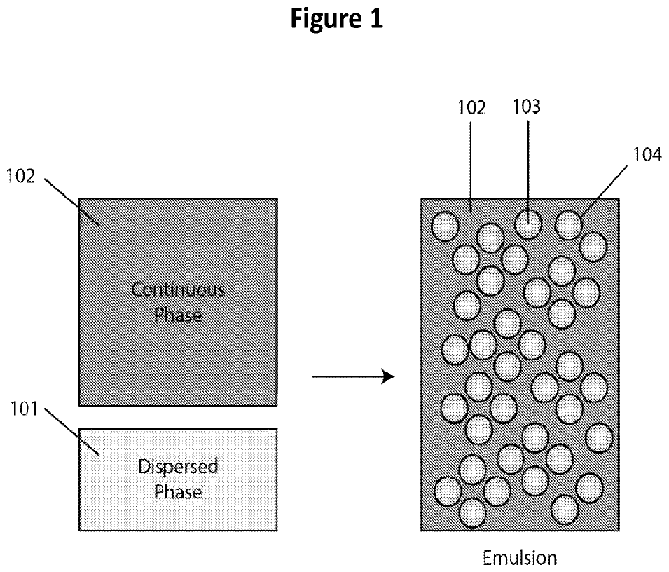

FIG. 1 illustrates formation of an emulsion. A dispersed phase liquid [101] is partitioned within a continuous phase liquid [102]. The resulting emulsion contains multiple dispersed phase partitions [103] solubilized in a bulk liquid of continuous phase [102]. Each partition [103] comprises a fractional volume of the original dispersed phase liquid and comprises an interface [104] between the dispersed phase liquid and the bulk continuous phase liquid.

The partitions [103] may range in cross-sectional diameter equating to a spherical volume ranging from 1 picoliter to 1 milliliter, e.g. 1 pL-1 mL, 1 pL-100 uL, 1 pL-10 uL, 1 pL-5 uL, 1 pL-1 uL, 1 pL-100 nL, 1 pL-10 nL, 1 pL-1 nL, 1 pL-100 pL, 1 pL-10 pL, 10 pL-1 mL, 10 pL-100 uL, 10 pL-10 uL, 10 pL-5 uL, 10 pL-1 uL, 10 pL-100 nL, 10 pL-10 nL, 10 pL-1 nL, 10 pL-100 pL, 100 pL-1 mL, 100 pL-100 uL, 100 pL-10 uL, 100 pL-5 uL, 100 pL-1 uL, 100 pL-100 nL, 100 pL-10 nL, 100 pL-1 nL, 1 nL-1 mL, 1 nL-100 uL, 1 nL-10 uL, 1 nL-5 uL, 1 nL-1 uL, 1 nL-100 nL, 1 nL-10 nL.

The partition interface [104] can be stabilized by a stabilizing agent, for example a surfactant. The stabilizing agent may impart physical stability to the droplet interface reducing the occurrence of partition coalescence and/or breaking. The stabilizing agent may also impart interface integrity resulting in controlled or reduced transit of molecules across the interface.

The dispersed phase liquid [101] may be a hydrophilic liquid, e.g., an aqueous liquid, a hydrophobic liquid, or a fluorophilic liquid. Depending on the composition of the dispersed phase liquid [101], the continuous phase liquid [102] may also be either a hydrophilic liquid, e.g., an aqueous liquid, a hydrophobic liquid, or a fluorophilic liquid. The continuous phase liquid [102] is substantially immiscible with the dispersed phase liquid to form an emulsion.

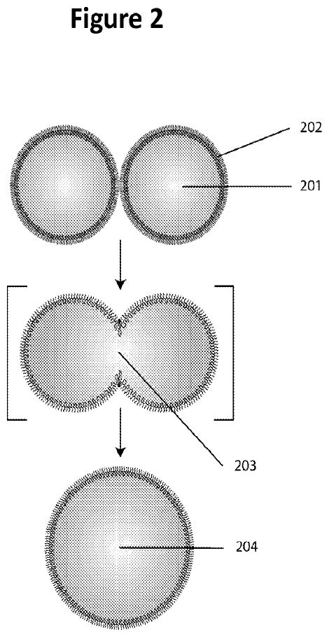

FIG. 2 is a schematic representation of the coalescence of two or more partitions [201] resulting in a single partition [204] with a post coalescence volume equal to the combined volume of the two or more starting partitions.

In partition coalescence, the two or more starting partitions [201] each comprise a dispersed phase liquid interior surrounded by a bulk continuous phase exterior. A partition interface [202] exists at the intersection of the dispersed phase and continuous phase liquids. The two or more starting partitions [201] may or may not have an interface [202] stabilized by a stabilizing agent. Suitable stabilizing agents may include surfactants.

In some instances, two or more starting partitions [201] may enter a state of high proximity and high energy resulting in complete loss of the continuous phase fluid separating the two or more partitions [201]. The loss of continuous phase fluid and a presence of surface energy result in destabilization of the partition interface [202] and fusion of the two or more partition interfaces [203], upon which the two or more partitions coalesce forming a resulting partition [204] of a volume equal to the combined volume of the two or more original partitions [201].

Without being bound by theory, it is thought that various phenomena may lead to the physical destabilization of emulsions: creaming, sedimentation, flocculation, phase inversion, Ostwald ripening, any or all of which may result in, or increase the probability of, coalescence. The mechanisms of instability depend on partition size, size distribution, amount and type of emulsifier, mutual solubility of the two phases, agitation, temperature, and pH. For example, coalescence results from the high surface free energy of the emulsion (ΔG), which is energetically unfavorable. As a result, the system drives towards reduction of the total interfacial energy (ΔA) meaning partitions tend to destabilize and go back to their unmixed state: ΔG=γΔA. Where γ denotes interfacial tension, and the system energy is almost always positive [McClements, 2015, Food Emulsions: Principles, Practices, and Techniques, Third Edition].

As shown in FIG. 2, in some cases, partitions coalesce as the interfacial film between the partitions drains, allowing for complete removal of the continuous phase barrier separating the two or more dispersed phase partitions. In this model, coalescence is dependent on at least the following two parameters, contact time and film drainage. When contact time exceeds film drainage time, meaning two or more partitions come into contact with each other long enough for all the oil to drain from between them, the partitions may rupture, resulting in coalescence. Film drainage is induced by the capillary pressure due the pressure difference between the dispersed and continuous phases. It may be slowed or prevented by the disjoining pressure resulting from van de Waals, steric, and electrostatic interactions between film surfaces.

Coalescence may be exacerbated mechanically by increasing the energy in the system or forcing the partitions into closer contact through temperature variations or shear forces.

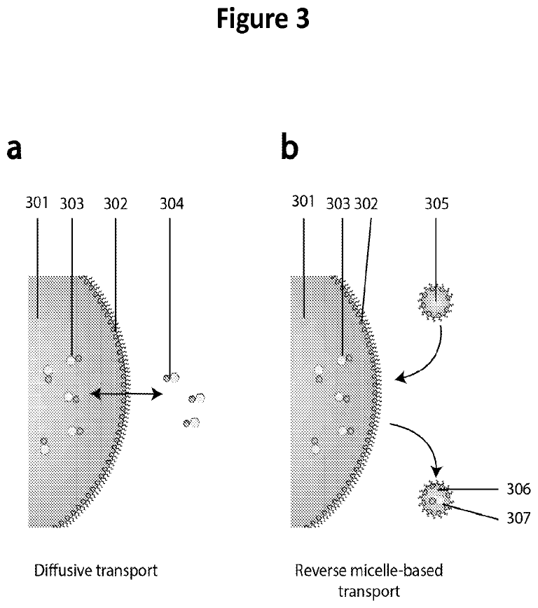

FIG. 3 shows a schematic representation of two modes of molecular transit across a partition interface [302]. Molecular transit may occur either from partition interior [301] to the continuous phase fluid or from the continuous phase fluid to the partition interior [301]. In some instances, transit may occur in both directions, and in some instances transit may prefer one of the two transit directions. The transit may exist in a static or dynamic equilibrium after a sufficient period of time to generate a negligible overall concentration gradient.

In a first instance, molecular transit is governed by diffusion. Molecules [303] solubilized in the partition interior [301] may diffuse across the partition interface [302] into the bulk continuous phase or from the bulk continuous phase across the partition interface [302] into the partition interior [301]. The resulting molecules in the bulk continuous phase [304] may then diffuse back across the partition interface into the droplet interior [301].

Molecules in the continuous phase [304] may transit the interface of any partition in the emulsion resulting in potential transit of molecules between partitions in the emulsion, that is, a molecule in one partition can move to a second partition.

In a second instance, molecular transit is governed by the formation of surfactant micelles [305] in the bulk continuous phase, their fusion with a partition interface, and the generation and release of new surfactant micelles back into the continuous phase. Micelles are an aggregate of surfactant molecules whose head groups are interacting in the micelle interior, in the case of a micelle in a hydrophobic solvent.

Surfactants embedded in the partition interface [302] and surfactants embedded in micelles [305] in the bulk continuous phase liquid are constantly exchanging in a dynamic equilibrium. In this dynamic equilibrium, a surfactant micelle [305] will fuse with the partition interface and the surfactants in the micelle will embed themselves in the partition interface [302]. Since there is a fixed surface area for surfactant molecules to exist, the fusion of the micelle will cause a displacement of surfactant molecules somewhere in the partition interface [302]. This displacement can result in the formation of a new surfactant micelle [306] with either the same or different surfactant molecules. This process is called reverse micelle formation.

Molecules [303] dissolved in the partition interior [301] may be captured in the interior of the newly formed micelle upon micelle formation. These micelle-captured molecules [307] are now effectively dissolved in the bulk continuous phase. Molecule-filled micelles [306] may then either remain permanently in the continuous phase or associate with any suitable partition in the emulsion.

In this instance molecular transit is not governed by the principles of diffusion but rather based on size and the relative kinetics of reverse micelle formation as well as surfactant concentration in the emulsion.

In some embodiments, decreasing temperature, increasing continuous phase viscosity, and reducing partition-partition proximity may reduce the efficiency of molecule transit from the interior to exterior of a partition or vice versa.

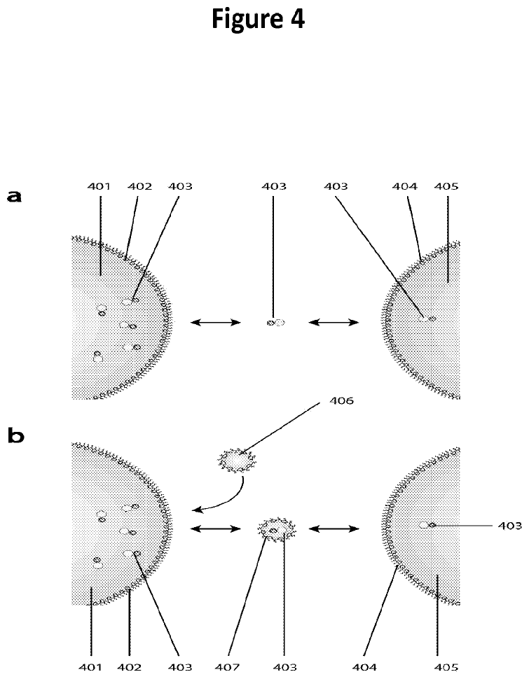

FIG. 4 shows another schematic representation of two modes of molecular transit between separate partitions in an emulsion, diffusion and micelle formation.

In a first instance, a first partition comprises a volume of dispersed phase liquid [401] within a bulk continuous phase. A partition interface [402] exists at the intersection of the dispersed phase liquid [401] and bulk continuous phase liquid. The partition interface [402] may or may not be stabilized by a stabilizing agent such as a surfactant. In the same emulsion, one or more additional dispersed phase partitions also reside in the bulk continuous phase. The additional dispersed phase partitions also comprise a dispersed phase interior [405] and a second partition interface [404]. Molecules residing in the first partition [403] may or may not transit across the first partition interface [402] into the bulk continuous phase liquid by diffusive transit. Molecules transited into the bulk continuous phase may then pass through the bulk continuous phase freely. Upon reaching the second partition, the continuous phase molecules [403] may then transit the second partition's interface [404] and enter the dispersed phased interior [405] of the second partition.

In a second instance, molecular transit is governed by the formation of surfactant micelles [406] in the bulk continuous phase. Micelles are an aggregate of surfactant molecules whose dispersed-phase soluble groups are interacting in the micelle interior.

Surfactants embedded in the partition interface [402] and surfactant embedded in micelles [406] the bulk continuous phase liquid are constantly exchanging in a dynamic equilibrium. In this dynamic equilibrium, a surfactant micelle [406] will fuse with the partition interface and the surfactants in the micelle will embed themselves in the partition interface [402]. Since there is a fixed surface area for surfactant molecules to exist, the fusion of the micelle can cause a displacement of surfactant molecules somewhere in the partition interface [402]. This displacement will result in the formation of a new surfactant micelle [407] with either the same or different surfactant molecules. This process is called reverse micelle formation.

Molecules [403] dissolved in the partition interior [401] may be captured in the interior of the newly formed micelle [407] upon micelle formation. These micelle-captured molecules [403] are now effectively dissolved in the bulk continuous phase. Molecule-filled micelles [403] may then either remain permanently in the continuous phase, associate with the first partition, or embed themselves in interface of a second partition [404] effectively releasing the molecules [403] from the first partition into the dispersed phase interior [405] of the second partition.

Molecular transit from droplet to droplet through diffusive or reverse micelle means over long periods of time will tend to reach an equilibrium state. This type of molecular transport may have an undesirable effect on the ability to use partitions as isolated reaction vessels since molecules able to transit through these means are no longer constrained to their original partition and, for example, detectable markers generated in one reaction vessel partition will not necessarily remain associated with the partition that generated it.

In some cases, decreasing temperature, increasing continuous phase viscosity, and reducing partition-partition proximity may reduce the efficiency of molecule transit from the inter to exterior of a partition or vice versa.

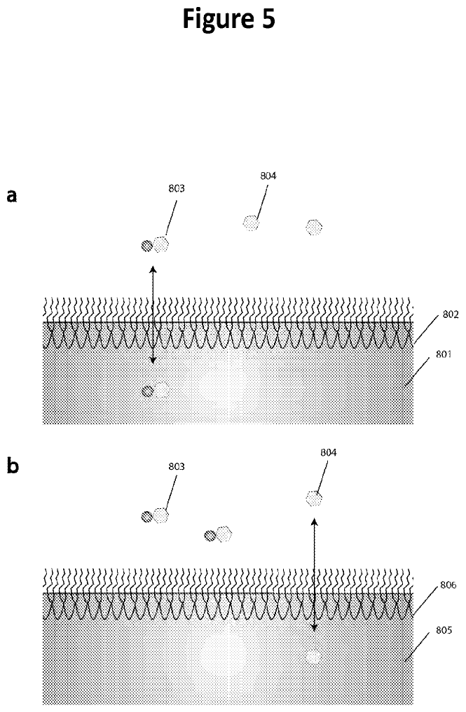

FIG. 5 shows an embodiment of surfactant cross-linking. Surfactant cross-linking refers to the formation of linkages [501 and 502] between surfactants embedded in a partition interface through the interaction and/or chemical reaction of one or more physically linked cross-linking agents.

Surfactants are typically amphiphilic molecules comprising two or more linked moieties soluble in either the continuous phase or dispersed phase fluidics. In the figure above moiety A and moiety B refer to groups of the surfactant that are soluble in opposing fluids. For example, if moiety A is preferentially soluble in the continuous phase then moiety B is preferentially soluble in the dispersed phase. For example, if moiety B is preferentially soluble in the continuous phase then moiety A is preferentially soluble in the dispersed phase.

Cross-linking can occur between the heads of surfactant molecules (i.e., the moiety that is preferentially soluble in the continuous phase and that is in contact with continuous phase), between the tails of surfactant molecules (i.e., the moiety that is preferentially soluble in dispersed phase and that faces the interior of the partition), between other parts of the surfactant molecules, or a combination thereof.

Cross-linking of surfactant molecules embedded in the partition interface results in the formation of a surfactant network at the interface that promotes retention of surfactant molecules at the surface, effectively increasing partition stability over those in which the surfactant particles are not cross-linked. This can help to retain the continuous phase film surrounding each partition even in applications with long contact time, elevated temperatures, and variations in pH, or in the presence of chemicals that would destabilize partitions in which the surfactant particles are not cross-linked. The cross-linking may be spontaneous or induced, may be formed using a one component system or a many component system, and the extent of cross-linking may vary from partial to full. In certain embodiments, the cross-linking moiety on a surfactant that interacts with another cross-linking moiety on another reactant, are moieties that are separate from the moieties A and B that provide the amphiphilic nature of the surfactant, i.e., they are additions to the head or tail of the surfactant and are not part of the head or tail.

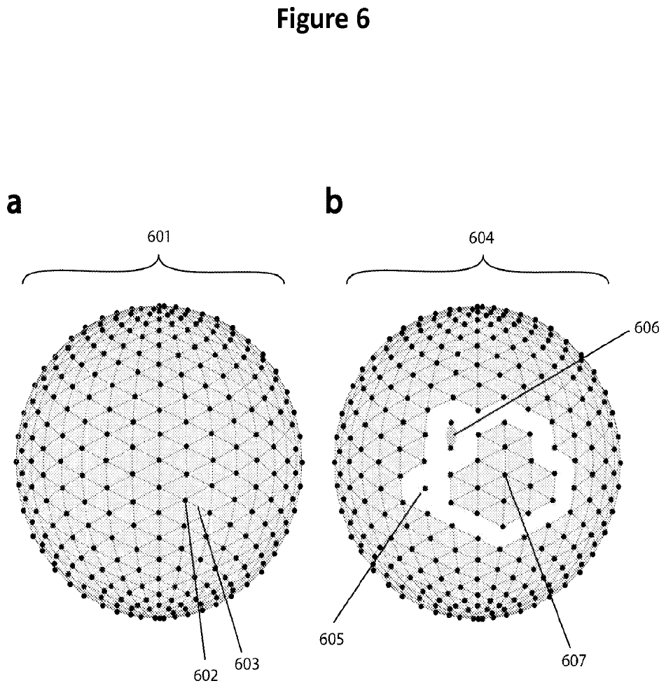

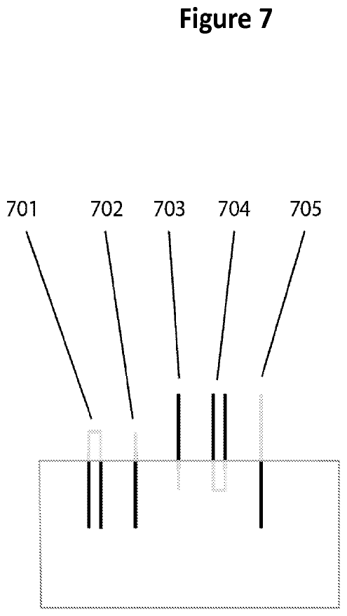

FIG. 6 shows the extent of cross-linking may vary from partial to full.

In FIG. 6a, full cross-linking has been achieved. In this embodiment, a partition [601] comprises a partition interface saturated with surfactant molecules [602]. Each surfactant molecule is linking to at least one other surfactant molecule through a cross-link [603] resulting in a connection between those surfactant molecules. Each surfactant molecule in the partition interface is connected to other neighboring surfactant molecules forming a fully interconnected surfactant network at the partition interface. Full or complete cross-linking occurs when 100% or substantially 100% of neighboring surfactant molecules of a partition interface are cross-linked together. In the case of FIG. 6a, each surfactant molecule has 5 neighbors, and complete cross-linking is achieved when all or substantially all of the surfactant molecules are bonded to all of their 5 neighbors. Additionally or alternatively, if some fraction of the surfactants in the surface are fluorescent, and since surfactants that are not crosslinked can move freely around the surface, once cross-linking is achieved those fluorescent surfactants would be fixed in place. In FRAP a spot on a sample is photobleached and then watched for dyes to move back into that photobleached area. In the case of reverse micelles, the whole droplet can be bleached and if reverse micelles are occurring the bleached surfactants will leave and then new fluorescent ones will come in. Timing of dyes moving back into a photobleached spot and/or timing of fluorescent molecules moving to an entire photobleached droplet can be used, in some cases compared with timing for droplets prepared without cross-linking (but otherwise the same). In certain embodiments, surfactant networks are 1-100, 10-100, 20-100, 30-100, 40-100, 50-100, 60-100, 70-100, 80-100, 90-100, 95-100, or 99-100% cross-linked.

In a separate embodiment, partial cross-linking has been achieved. In an example of this embodiment (FIG. 6b), a partition [604] comprises a partition interface saturated with surfactant molecules. The number of linkages between surfactants in the partition interface ranges from 0 linkages (0% complete) to a maximum number of linkages based on the number of surfactant molecules neighboring the surfactant (100% complete). A surfactant molecule may have no linkages to neighboring surfactants [605]. A surfactant network may be realized though cross-linking of neighboring surfactants. However, in some cases the extent of that network may not encompass the entirety of the partition interface. This may range in surfactant ‘rafts’ of size from, e.g., 2 surfactants [606] to larger ‘rafts’ of more than two surfactants [607].

FIG. 7 shows that surfactants embedded in the partition interface may vary in physical geometry. This is a non-limiting list of surfactant geometries.

In one example the surfactant is relatively linear molecule with one portion of the surfactant soluble in either the continuous or dispersed phase fluid and the other portion of the surfactant soluble in the opposing fluid [702, 703, and 705]. One surfactant geometry may include a surfactant with a large portion soluble in the dispersed phase and a small portion soluble in the continuous phase [702]. A second surfactant geometry may include a surfactant with a small portion soluble in the dispersed phase and a large portion soluble in the continuous phase [703]. A third surfactant geometry may include equally sized portions soluble in their respective phases [705]. These portions may either be large or small.

In a second example, the surfactant is a bent molecule with two or more external regions soluble in either the continuous or dispersed phase fluids and an internal region soluble in the opposite phase [701 and 704]. These surfactants may be referred to as gemini surfactants. One surfactant geometry may include a surfactant with external portions soluble in the dispersed phase and internal portions soluble in the continuous phase [701]. A second surfactant geometry may include a surfactant with internal portions soluble in the dispersed phase and external portions soluble in the continuous phase [705].

Any surfactant geometry may be utilized for surfactant cross-linking. Combinations of surfactant geometries may be utilized for surfactant cross-linking. Generally it is preferred that a linkage moiety is attached to a portion of the surfactant molecule that will be accessible to cross-linking components or conditions; for example, a linkage moiety, e.g. biotin, can be attached to the hydrophilic head portions of surfactant molecules and one or more components that initiate or promote cross-linking of the linkage moieties, e.g., an intermediate linkage moiety such as streptavidin, may be present in aqueous dispersed phase, into which the hydrophilic head will preferably locate when partitions form. In another example, a linkage moiety is attached to the hydrophobic tail of the surfactant molecule that will be accessible to cross-linking components in, e.g., hydrophobic continuous phase, so that such components are in the continuous phase to which the tail groups will be exposed. Other combinations, e.g., for hydrophilic continuous phase and hydrophobic dispersed phase, are readily apparent.

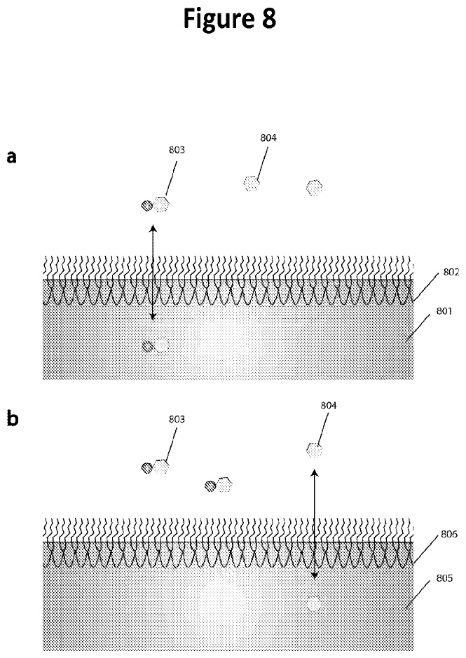

FIG. 8 shows an embodiment of control of molecular transit though modification of the chemical properties of the partition interface. Modulation of the chemistry at the partition interface will selectively reduce transit of molecules with non-complementary chemistries across the partition interface.

In one example (FIG. 8a), a partition comprises a dispersed phase interior [801], a continuous phase exterior, and a highly charged cross-linked partition interface [802]. Molecules of either hydrophobic [804] and or hydrophilic [803] nature may exhibit differential transport across the partition interface. In particular, a hydrophobic molecule is highly unlikely to cross the partition interface due to the highly hydrophilic nature of the charged cross-linked partition. A hydrophilic molecule may be more or less likely to cross, depending on its charge, either a full charge (ionic) or a partial charge (due to differing electronegativities of constituent atoms); in either case, if the charge of a molecule is the same as the charge of the cross-linked partition, it will be less likely to cross due to electrostatic repulsion, whereas if the charge of a molecule is opposite of the charge of the cross-linked partition it will be more likely to cross due to electrostatic attraction.

In a second example (FIG. 8b), a partition comprises a dispersed phase interior [805], a continuous phase exterior, and a highly hydrophobic partition interface [806]. Molecules of either hydrophobic [804] and or hydrophilic [803] nature may exhibit differential transport across the partition interface. In particular, hydrophobic molecules will preferentially pass through the interface compared to hydrophilic molecules.

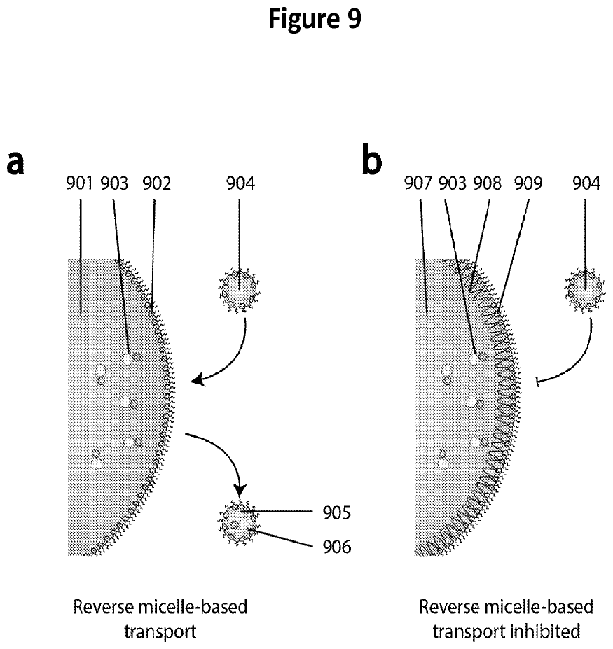

FIG. 9 shows an example of reduction of molecular transit by inhibition of reverse micelle formation. In this instance, molecular transit is governed by the formation of surfactant micelles [906] in the bulk continuous phase. Micelles are an aggregate of surfactant molecules whose dispersed-phase soluble groups are interacting in the micelle interior.

FIG. 9a shows an example of a surfactant interface that is not cross-linked. Surfactants embedded in the partition interface [902] and surfactant embedded in micelles [904] the bulk continuous phase liquid are constantly exchanging in a dynamic equilibrium. In this dynamic equilibrium, a surfactant micelle [904] will fuse with the partition interface and the surfactants in the micelle will embed themselves in the partition interface [902]. Since there is a fixed surface area for surfactant molecules to exist, the fusion of the micelle will cause a displacement of surfactant molecules somewhere in the partition interface [902]. This displacement will result in the formation of a new surfactant micelle [905] with either the same or different surfactant molecules. This process is called reverse micelle formation.

Molecules [903] dissolved in the partition interior [901] may be captured in the interior of the newly formed micelle [905] upon micelle formation. These micelle-captured molecules [903] are now effectively dissolved in the bulk continuous phase. Molecule-filled micelles [905] may then either remain permanently in the continuous phase, associate with the first partition, or embed themselves in interface of a second partition effectively releasing the molecules [903] from the first partition into the dispersed phase interior of the second partition.

Molecular transit from droplet to droplet through diffusive or reverse micelle means over long periods of time molecules will tend to reach an equilibrium state. This type of molecular transport may have a negative effect on the ability to use partitions as isolated reaction vessels since molecules able to transit through these means are no longer constrained to their original partition and detectable markers generated in one reaction vessel partition will not necessarily remain associated with the partition that generated it. In addition, as a molecule, e.g., reactant in a partition is used up in a chemical reaction, the concentration gradient for diffusion of external molecules into the partition is greater and can cause further influx of the molecule into the partition.

Inhibition of reverse micelle formation by the physical linkage of surfactant molecules may effectively decrease or eliminate molecular transit by this process (FIG. 9b). In this case, a partition comprises a dispersed phase interior [907], a partition interface [909], cross-links between the surfactants of the interface [908], and molecules [903] dissolved within the dispersed phase interior [907]. Since the surfactants have formed a surfactant network at the partition interface, continuous phase-dissolved surfactant micelles [904] may not embed themselves within the partition interface due to space constraints and micelles from the partition interface may not form new micelles and exit into the continuous phase as they are physically constrained to the partition interface by their neighboring, connected surfactants.

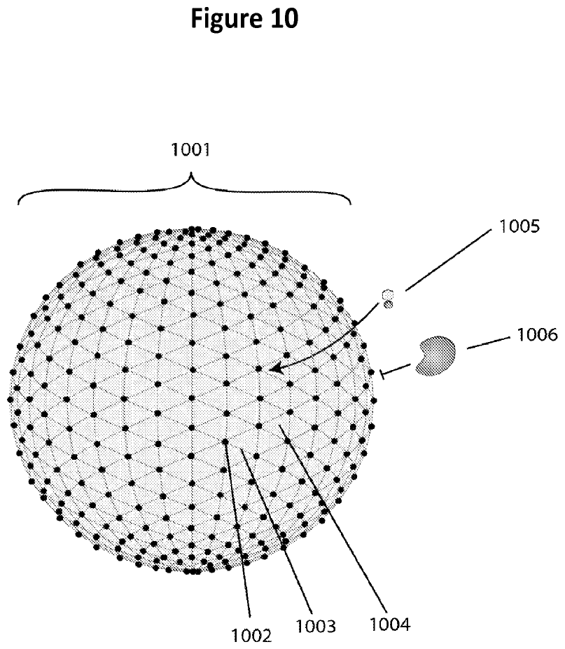

FIG. 10 shows an example of controlled molecular transit through size-based limitations of surfactant network porosity. In this example, the specific geometrical formation of a cross-linked surfactant network yields a network of defined porosity acting as a molecular sieve for controlled molecular transit.

A partition [1001] comprises a dispersed phase interior, a continuous phase exterior, a partition interface, surfactants embedded in the interface [1002], cross-links between the neighboring surfactants [1003], and pores [1004] of defined size based on the chemical and physical properties of the surfactant cross-links. In this example, molecules of a volume suitable [1005] to pass through those pores [1004] may enter and exit the partition based on diffusion principles. Molecules of a volume not suitable [1006] to pass through pores [1004] may not enter nor exit the partition.

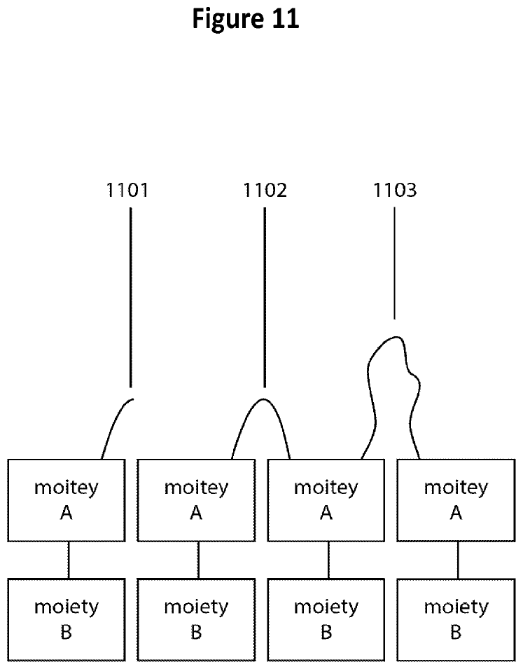

FIG. 11 shows effects of varying linker size, i.e., cross-linker length.

Cross-linker sizes, i.e., lengths, may range from small to large, i.e., short to long. As a result, cross-linker length may alter properties of cross-linking at the partition interface. Cross-linkers of too short a length [1101], may not effectively reach neighboring surfactant molecules resulting in inhibition of cross-link formation. Cross linkers of too long of length [1103] may cross-link with more surfactant molecules resulting in a loose surfactant network.

Therefore, cross-linker lengths can be tailored to suitable sizes [1102] for specific cross-linking applications. In certain embodiments, the cross-linker length, e.g., the length of the final linkage between two surfactant molecules formed by linking a linkage moiety on each molecule, in some cases directly and in some cases indirectly via an intermediate linkage moiety, is selected to be 0.1-100×, 0.1-50×, 0.1-30×, 0.1-20×, 0.1-10×, 0.1-5×, 0.1-3×, 1-100×, 1-50×, 1-30×, 1-20×, 1-10×, 1-5×, or 1-3× the longest dimension of the head group of the surfactant, or of the longest dimension of the tail group of the surfactant. In general herein, “head group” refers to the hydrophilic portion of the surfactant and “tail group” refers to the hydrophobic portion. To simplify terminology, this nomenclature is used for both hydrophobic dispersed phase partitions in hydrophilic continuous phase (hydrophilic head group faces out from partition to continuous phase, hydrophobic tail group faces in to hydrophobic dispersed phase) and hydrophilic dispersed phase partitions in hydrophobic continuous phase (hydrophilic head group faces inward from partition to hydrophilic dispersed phase inside the partition, hydrophobic tail group faces outward to hydrophobic continuous phase). In certain embodiments, the cross-linker is 0.1-100 nm, 0.1-50 nm, 0.1-30 nm, 0.1-20 nm, 0.1-10 nm, 0.1-5 nm, 0.1-3 nm, 1-100 nm, 1-50 nm, 1-30 nm, 1-20 nm, 1-10 nm, 1-5 nm, or 1-3 nm in length.

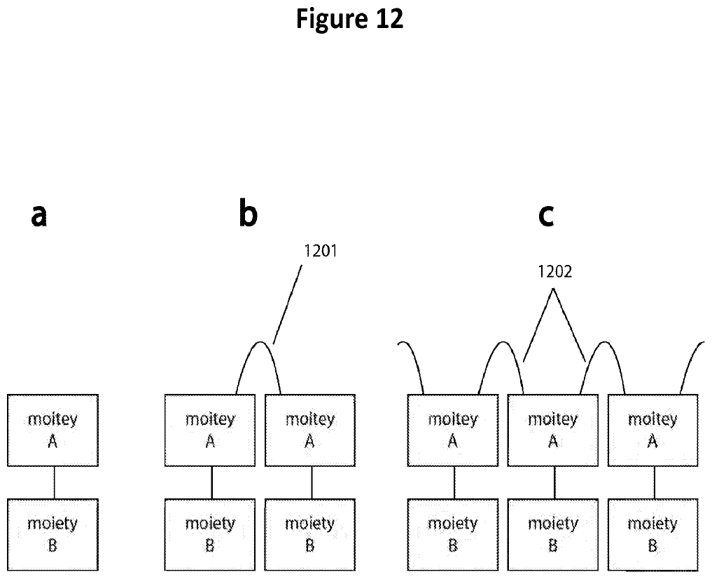

FIG. 12 shows effect of cross-linking agent number, i.e., number of linkage moieties per surfactant molecule, on extent of cross-linking. In the case of surfactants containing no cross-linking agent, i.e., no linkage moieties, no surfactant linkages may be formed. See FIG. 12a. In the case of surfactants bearing a single cross-linking agent, i.e., single linkage moiety, [1201], a maximum of two neighboring surfactants may be linked. See FIG. 12b. In the case of surfactants bearing more than one cross-linking agents [1202], multiple neighboring surfactants may be cross-linked throughout the partition interface. See FIG. 12c.

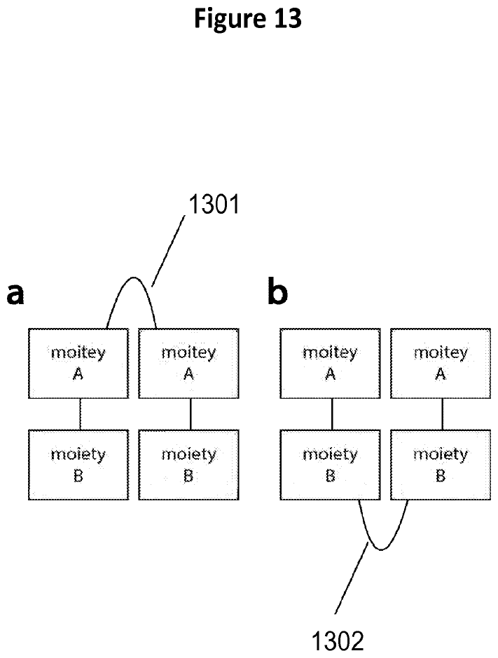

FIG. 13 shows head-to-head cross-linking and tail-to-tail cross-linking of surfactant moieties; moiety A is a head group of a surfactant and moiety B is the tail group. FIG. 13a illustrates head-to-head cross-linking by a cross-linker (1301); FIG. 13b illustrates tail-to-tail cross-linking by a cross-linker (1302).

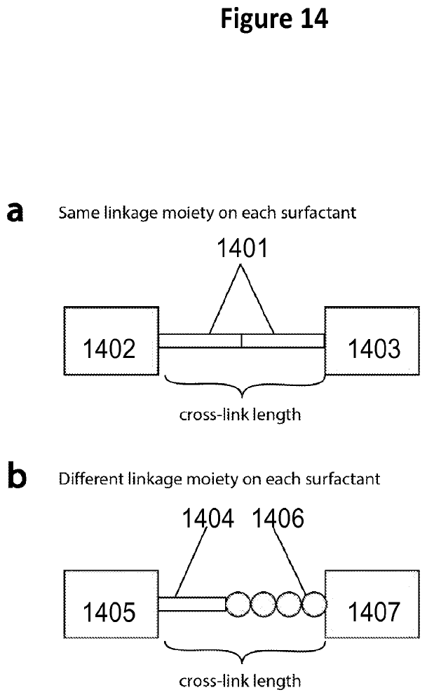

FIG. 14 shows direct cross-linking between linkage moieties on two different surfactant molecules. FIG. 14a shows the same linkage moiety (1401) on each surfactant moiety (1402 and 1403), where the surfactant moieties 1402 and 1403 can be the same or different. FIG. 14b shows a first linkage moiety 1404 on a first surfactant moiety 1405 and a second linkage moiety 1406 on a second surfactant moiety 1407, where the first and second linkage moieties are different and where the first and second surfactant moieties may be the same or different. The attachment between the two linkage moieties may be covalent or noncovalent.

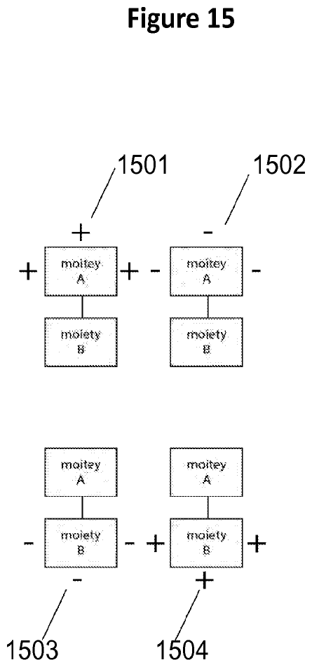

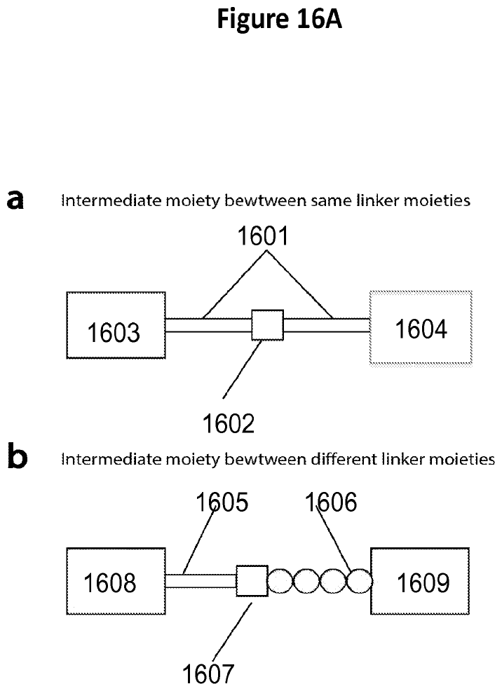

FIG. 15 shows cross-linking of two surfactant moieties by ionic bond. In some cases, the head groups (moiety A) of the surfactant moieties 1501 and 1502 have opposite charges. In some cases the tail groups (moiety B) of the surfactant moieties 1503 and 1504 have opposite charges. The charges may be present in the surfactant moieties as is, or may be created and/or enhanced by attachment of one or more charged groups to the surfactant.

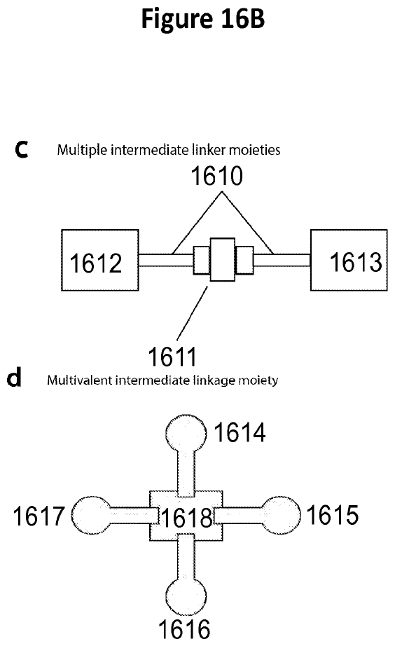

FIG. 16 shows indirect cross-linking between two surfactant moieties via one or more intermediate linkage moieties. FIG. 16a shows first and second surfactant moieties 1603 and 1604, which can be the same or different, with linkage moieties 1601 indirectly cross-linked via intermediate linkage moiety 1602. FIG. 16b shows a first surfactant moiety 1608 with a first linkage moiety 1605 attached via intermediate linkage moiety 1607 to second linkage moiety 1606 of second surfactant moiety 1609. FIG. 16c shows first and second surfactant moieties 1612 and 1613, each with linkage moiety 1610, where multiple intermediate moieties 1611 attach linkage moieties 1610. FIG. 16d shows an intermediate linkage moiety, 1616, that is configured to attach to multiple surfactant linkage moieties; in this example, there are first surfactant moiety 1614 with a first linkage moiety, second surfactant moiety 1615 with a second linkage moiety, third surfactant moiety 1616 with a third linkage moiety, fourth surfactant moiety 1617 with a fourth linkage moiety, where the first, second, third, and fourth surfactant moieties are cross-linked via attachment of their respective linkage moieties to intermediate linkage moiety 1616. The first, second, third, and fourth surfactant moieties may be the same, or one or more of them may be different from the others, or any combination thereof. The first, second, third, and fourth linkage moieties may be the same, or one or more of them may be different from the others, or any combination thereof.

Whether partitions are pooled into some type of container or are kept in motion inside of a fluid pathway, in general, the goal of the compositions and methods disclosed herein is to improve the stability of partitions in an emulsion by strengthening the partition interface. This increase in stability can result in decreased partition coalescence. Alternatively or additionally, increased stability inhibits the flow of molecules out of the dispersed phase and into the continuous phase, from the continuous phase into the dispersed phase, or from one partition to another. The exchange of dispersed constituents from or into the continuous phase from partition to partition can be undesirable as it can impact or obscure the measurement of a particular analyte in each partition. Compositions and methods disclosed herein may, in some cases, help enable the selective transport of particular types of molecules between partitions, from continuous phase to dispersed phase, and/or from dispersed phase to the continuous phase. In some circumstances, the exchange of dispersed constituents from or into the continuous phase or from partition to partition is desired, e.g., it can improve the measurement of a particular analyte in each droplet.

Herein are disclosed compositions and methods for improving partition stability by cross-linking surfactant molecules after they form at the interface of the partitions, and/or during the formation of the interface. The cross-linking of the surfactants forms a surfactant network around each partition to promote retention of surfactant molecules at the surface, increasing partition stability over those in which the surfactant particles are not cross-linked. This helps to retain the continuous phase film surrounding each partition even in applications with long contact time, elevated temperatures, and variations in pH, or in the presence of chemicals that would destabilize partitions in which the surfactant particles are not cross-linked. The cross-linking may be spontaneous or induced, may be formed using a one component system or a many component system, and the extent of cross-linking may vary from partial to full.

In general, cross-links between surfactant molecules form during and/or after partition formation, e.g., at a droplet generator. Surfactant with linkage moieties attached and/or with a portion or portions amenable to cross-linking, can be present in dispersed phase, in continuous phase, or in both prior to partition formation. In certain embodiments, surfactant with linkage moieties attached is substantially all in continuous phase prior to partition formation, and for convenience the process will be described for this embodiment, but it will be appreciated that all combinations are encompassed by the methods and compositions provided herein. Surfactant for stabilizing dispersed phase partitions can be present in the continuous phase before partition formation, e.g., surfactant molecules with linkage moieties attached. Previous to formation of partitions, dispersed phase may be present as a programmed emulsion, that is, as a relatively large bolus of dispersed phase surrounded by a layer of continuous phase, with a layer of surfactant molecules at the interface. See, e.g., US Patent Application Publication No. 20200030794. In certain embodiments the surfactant of the programmed emulsion is not cross-linked. Thus, surfactant for stabilizing dispersed phase partitions in a continuous phase can form the basis for forming cross-links between surfactants in partitions formed in the continuous phase. Continuous phase with cross-linking surfactant and dispersed phase are initially separate. When partitions of dispersed phase form in the continuous phase to produce an emulsion, a surfactant interface forms on the partitions made up entirely, or substantially entirely, of surfactant molecules from the continuous phase. The conditions during partition formation and/or after formation are such that the linkage moieties on the surfactant molecules supplied by the continuous phase form cross-links between surfactant molecules. Formation of cross-links may be initiated and/or promoted in a variety of ways, as described herein.

In certain embodiments conditions in continuous phase that contains surfactant molecules with linkage moieties for formation of cross-linked surfactant networks are such that cross-links do not form, or do not substantially form, while the surfactant is in the continuous phase separate from the dispersed phase. Alternatively, conditions can be such that cross-links form, but are of a structure such that the cross-links can break and re-form when the surfactant is in contact with dispersed phase. In certain embodiments, e.g., photo cross-linking, conditions are altered when emulsion forms to promote formation of cross-links. In some cases, different surfactants with different linkage moieties can be segregated in the continuous phase, e.g., added separately to the continuous phase. Though there will be some migration between micelles of surfactant in the continuous phase, the timing of addition and/or conditions of the continuous phase before partition formation can be such that such migration is minimized.

Cross-link formation during and/or after formation of partitions can be induced and/or promoted in any suitable manner. In certain embodiments, dispersed phase contains one or more components, and/or establishes one or more conditions, that induce and/or promote cross-link formation, either direct or indirect; the components and/or conditions can, in certain embodiments, not be present or not be substantially present in continuous phase and/or not be present before partition formation. For example, in certain embodiments dispersed phase contains one or more components that initiate a reaction between linkage moieties of separate surfactants to form a direct cross-link. For example, as described below, some surfactant molecules can have a first linkage moiety that is an azide and other surfactant molecules can have a second linkage moiety that is an alkyne. The surfactant molecules are present in the continuous phase but cross-links don't form until the surfactant molecules encounter dispersed phase, which contains components necessary to induce and/or promote copper or copper-free alkyne-azide cycloaddition. In certain embodiments, dispersed phase contains one or more intermediate linkage moieties that are capable of forming bonds with linkage moieties of surfactant molecules to form indirect cross-links between surfactant. In some cases, one or more components are present in the dispersed phase and/or continuous phase that promote bond formation between the intermediate linkage moiety or moieties and the surfactant linkage moieties. An example of an intermediate linkage moiety is a biotin-binding moiety, such as streptavidin. Such a moiety can be present in the dispersed phase, while continuous phase contains surfactant molecules with biotin attached as a linkage moiety. Upon formation of partitions, the biotin-binding intermediate linkage moieties interact with the biotin attached to surfactant molecules to produce cross-links between the surfactant linkage moieties and the intermediate linkage moiety, to produce a surfactant cross-link network. In some cases, conditions are altered through external methods to induce and/or promote bond formation between linkage moieties and/or between linkage moieties and intermediate moieties. An example is the use of photo-activated cross-links, where cross-linking does not occur or does not substantially occur until linkage moieties are exposed to the appropriate light. In some cases, conditions present in dispersed phase can initiate and/or promote bond formation between linkage moieties. For example, the pH of the dispersed phase can be such that ion formation in linkage moieties is favored and ionic interactions between linkage moieties bond the moieties.

In certain embodiments, different surfactant molecules have attached the same linkage moiety. In certain embodiments, a first surfactant molecule has a first linkage moiety attached and a second surfactant molecule has a second linkage moiety attached, where the first and second linkage moieties are different. In certain embodiments, cross-linking is direct, that is, surfactant linkage moieties form a bond between each other; in certain embodiments, cross-linking is indirect, that is, surfactant linkage moieties form bonds with one or more intermediate moieties. In the latter case, in certain embodiments a single intermediate moiety may be used and in other embodiments a plurality of intermediate moieties may be used. Linkage bonds may be covalent or non-covalent.

Linkage moieties may be attached to the hydrophilic or polar portion of surfactant molecules, also referred to as a head or head group herein, or to the hydrophobic or non-polar portion of surfactant molecules, also referred to as a tail or tail group herein, or a combination thereof.

There are many methods for cross-linking surfactant molecules including, but not limited to, mechanical meshing, ionic interactions, chemical cross-linking, photo cross-linking, and ligand binding interactions.

### Ionic Interaction

In certain embodiments, a mixture comprising surfactant molecules with at least two different ionic natures is used. A first surfactant comprises a region of positive charge while a second surfactant comprises a region of negative charge. Generally, the charged regions will be on the head groups. When the surfactant molecules associate at the droplet interface the oppositely charged regions form a polyelectrolyte network at the droplet interface. The number of surfactant molecules included in the polyelectrolyte network at the droplet interface is dependent in part on the length of the charged regions and the number of different surfactant molecules those regions may interact with. Charge may be imparted by addition of groups to the head group, or from the nature of the head group without modification; in either case, the linkage moiety can be considered the charge and/or charged group itself.

In certain embodiments, a single surfactant with an aqueous moiety bearing either a highly positive or negative charge is used. Crosslinking occurs in the aqueous phase through counter-ions forming salt-bridge networks between the surfactant molecules at the partition interface.

### Chemical/Photo Cross-Linking

In certain embodiments, cross-linking between multiple surfactants, e.g., fluorosurfactants, may be achieved chemically using a cross-linking agent that is chemical or photo-activated that may either by linked to the surfactant, e.g., fluorosurfactant, or free in solution that is reactive primarily with a moiety linked to the surfactant molecule.

A covalent interaction between two or more surfactants, e.g., between surfactant linkage moieties, or between a cross-linking agent and the surfactant, e.g., between intermediate linkage moieties and surfactant linkage moieties, may be generated by any suitable chemistry. The compositions and methods disclosed herein can result in formation of surfactant networks that result in improved partition stability using one or more available chemical strategy. Chemical strategies include but are not limited to reactions involving the following functional groups: NHS ester, maleimide, squarane, alkyne-azide click-chemistry or analogous methods, and bioconjugation reactions (including reactions between amino acids such as lysine, cysteine, tyrosine with reactive groups as detailed, e.g., in Koniev, O., Wagner, A, Chem. Soc. Rev., 44, 5495 (2015)). In some embodiments, these chemistries are biologically compatible.

In certain embodiments, cross-linking is performed between two or more different surfactant molecules through the use of amine reactive chemistry. One example is by generating a first surfactant with an aqueous moiety composed of a pluronic-(polyoxy-hydrocarbon block copolymers) or jeffamine-family (amino polyethylene glycol block copolymers) polymer bearing water-accessible amine functional groups and a second surfactant with an aqueous moiety composed of a PEG-family polymer. See, e.g., U.S. Patent Application Publication No. 20180112036. By modifying the second surfactant with NHS ester functional groups and combining the second surfactant with the first surfactant in an aqueous environment, the NHS ester functional moieties form covalent linkages to the amine groups present in the first surfactant.

In certain embodiments, cross-linking between surfactants may be performed through a DNA-crosslinking mechanism. By linking short complementary oligonucleotides to the surfactant molecules, an inter strand cross-linking agent may be used to cross-link two or more pieces of oligonucleotide effectively cross-linking their attached surfactants. The cross-linking agent may be attached to the oligonucleotides prior to droplet formation or be free in the aqueous phase and react with the surfactants after partition formation. In certain embodiments, a single-stranded oligonucleotide intercalator is linked to the first surfactant. By linking a short oligonucleotide to the second surfactant and combining the two surfactants together in a partition interface, the two surfactant molecules will be linked forming a molecular network at the partition interface. In certain embodiments, a surfactant is generated with a single- or double-stranded oligonucleotide linked to it. By including a multifunctional cross-linking agent like a bi- or tri-functional crosslinker, the surfactants are cross-linked upon partition formation. Any suitable oligonucleotide cross-linking agent may be used in these and other embodiments. Examples of oligonucleotide cross-linking agents include but are not limited to 5F-203, 4′-Aminomethyltrioxsalen, 8-methoxypsoralen, Angelicin, Bifunctional aldehydes, Carboplatin, Carmustine, Chlorambucil, Cryptolepine, Cyclophosphamide, Fotemustine, Melphalan, Mitocin C, Mitoxantrone, Nitrous acid, Procarbazine, Psoralen, S)-tert-Butyl 1-(chloromethyl)-5-hydroxy-1H-benzo[e]indole-3(2H)-carboxylate, Treosulfan, Trioxsalen.

In certain embodiments, cross-linking between surfactants may be performed though a peptide-surfactant cross-linking mechanism. By linking a peptide to the first surfactant and a peptide reactive group to a second surfactant, upon reaction the surfactant molecules will be covalently cross-linked. In certain embodiments, a peptide is linked to the surfactant and a multifunctional interpeptide cross-linking agent may be used to cross-link one or more peptides. The cross-linking agent may be attached to the peptides prior to partition formation or be free in the dispersed phase, e.g., aqueous phase and react with surfactant-linked peptides after partition formation.

In certain embodiments, the aqueous moiety of the surfactant may be modified with sulfohydryl groups that upon entering an oxidative environment form inter-surfactant cross-links through a disulfide bond.

In certain embodiments, a first surfactant may be modified to have one or more alkyne functional groups and a second surfactant may be modified to have one or more azide functional groups. Upon entering the aqueous environment after droplet partitioning, the surfactants may be cross-linked using copper or copper-free alkyne-azide cycloaddition (‘click’) chemistry.

In certain embodiments, either a multifunctional alkyne or azide cross-linking agent may be located in the aqueous phase that reacts with surfactant molecules bearing the complementary chemistry upon partition formation.

In photo cross-linking, as is known in the art, linkage moieties on surfactant molecules contain a photo-reactive element, that upon exposure to appropriate light, forms a bond with another element, for example another photo-reactive element, which can be on another surfactant linkage moiety or on an intermediate linkage moiety In certain embodiments the photo-reactive elements are attached to peptide linkers that are attached to surfactant molecules; the use of such linkers allows control of cross-link length, e.g., by controlling the number of amino acid residues in the peptide, and/or cross-link hydrophobicity or hydrophilicity, including charge, by choosing appropriate amino acid residues for the desired effect. This is true in general in any embodiment in which peptide linker are used. Suitable photo-reactive elements are known in the art, and include, without limitation, aryl azides, azido-methyl-coumarins, benzophenones, anthraquinones, certain diazo compounds, diazirines, and psoralen derivatives. The light to activate cross-linking may be any suitable light, such as visible light or UV light.

### Ligand Binding

In some embodiments cross-linking is achieved through one or more ligand-receptor interaction. Either the ligand or the receptor may be linked to the surfactant or a multifunctional receptor or ligand may be present in the dispersed phase, or both.

Any suitable combination of ligand and receptor may be used. Ligand binding examples may include but are not limited to peptide-peptide, peptide-protein, peptide-small molecule, peptide, oligonucleotide, protein-small molecule, protein-nucleic acid, nucleic acid-small molecule, nucleic acid-nucleic acid interactions.

Examples of these include but are not limited to split inteins, spy catcher-spy tag, streptavidin-strep tag, antibody-epitope (like myc and anti-myc, FLAG and anti-flag, etc.) nucleic acid hairpins and ligation, his tag-nickel or cobalt, heparin-heparin binding proteins, poly N-acetyl glucosamine-wheat germ agglutinin, MBP-maltose or amylose.

In some embodiments, cross-linking is achieved through the conjugation of one or more biotin moieties to the surfactant and the subsequent cross-linking of one or more surfactants through biotin-binding moiety, e.g., streptavidin, -biotin interaction in the aqueous phase. When streptavidin is used, its multivalent nature gives it the ability ability to link up to 4 surfactant molecules.

Any suitable biotin-binding moiety may be used. In certain embodiments, a streptavidin or streptavidin derivative is used. Streptavidin is a well-known and studied biotin-binding moiety. Streptavidin is a protein that shows considerable affinity for biotin, a 244 Dalton co-factor that plays a role in multiple prokaryotic and eukaryotic biological processes. Streptavidin and other biotin-binding proteins, including avidin and their derivatives, have the ability to bind up to four biotin molecules. Streptavidin derivatives can be engineered to have 0, 1, 2, 3, or 4 biotin binding sites. An engineered form of streptavidin has been generated displaying a 10-fold slower dissociation, a 2-fold slower association rate, and higher thermal and mechanical stability [Chivers, 2010, Nature Methods].

The highly specific interaction of streptavidin with biotin (Kₐ=10¹⁵ M⁻¹) is a useful tool in biological assays. The protein-ligand complex demonstrates extraordinarily high stability including resistance to organic solvents, denaturants, detergents, proteolytic enzymes, as well as resistance extreme temperatures (e.g., >100° C.) and pH (e.g., 4-11)

The N and C termini of the 159 residue full-length protein are processed to give a shorter ‘core’ streptavidin, usually composed of residues 13-139; removal of the N and C termini is necessary for the high biotin-binding affinity. The secondary structure of a streptavidin monomer is composed of eight antiparallel β-strands, which fold to give an antiparallel beta barrel tertiary structure. A biotin binding-site is located at one end of each β-barrel. Four identical streptavidin monomers (i.e. four identical β-barrels) associate to give streptavidin's tetrameric quaternary structure. The biotin binding-site in each barrel consists of residues from the interior of the barrel, together with a conserved Trp120 from neighboring subunit. In this way, each subunit contributes to the binding site on the neighboring subunit, and so the tetramer can also be considered a dimer of functional dimers.

The numerous crystal structures of the streptavidin-biotin complex have shed light on the origins of the remarkable affinity. Firstly, there is high shape complementarity between the binding pocket and biotin. Secondly, there is an extensive network of hydrogen bonds formed to biotin when in the binding site. There are eight hydrogen bonds directly made to residues in the binding site (the so-called ‘first shell’ of hydrogen bonding), involving residues Asn23, Tyr43, Ser27, Ser45, Asn49, Ser88, Thr90 and Asp128. There is also a ‘second shell’ of hydrogen bonding involving residues that interact with the first shell residues. However, the streptavidin-biotin affinity exceeds that which would be predicted from the hydrogen bonding interactions alone, suggesting another mechanism contributing to the high affinity. The biotin-binding pocket is hydrophobic, and there are numerous van der Waals force-mediated contacts and hydrophobic interactions made to the biotin when in the pocket, which is also thought to account for the high affinity. In particular, the pocket is lined with conserved tryptophan residues. Lastly, biotin binding is accompanied by the stabilization of a flexible loop connecting B strands 3 and 4 (L3/4), which closes over the bound biotin, acting like a ‘lid’ over the binding pocket and contributing to the extremely slow biotin dissociation rate.

Avidin in contrast to streptavidin shares ˜30% sequence identity to streptavidin, but almost identical secondary, tertiary and quaternary structure. In contrast to streptavidin, it is glycosylated, positively charged, has pseudo-catalytic activity (it can enhance the alkaline hydrolysis of an ester linkage between biotin and a nitrophenyl group) and has a higher tendency for aggregation. Also, streptavidin is the better biotin-conjugate binder; avidin has a lower binding affinity than streptavidin when biotin is conjugated to another molecule, despite avidin having the higher affinity for free, unconjugated biotin. Because streptavidin lacks any carbohydrate modification and has a near-neutral pl, it has the advantage of much lower nonspecific binding than avidin.

In certain embodiments, split receptors may be used that reform upon binding their intended ligand. One example includes half of a metal chelator on a branch so that a couple of branches come together to chelate a metal ion. This may be achieved by many methods including but not limited to nucleic acids bearing modified base pairs that require metal ions like copper for hybridization.

### Two Part Cross-Linking

In another embodiment, a split surfactant is utilized that upon formation into the full surfactant at the partition interface, stabilizes the partition. In this embodiment, one part of the split surfactant is soluble in the continuous phase while a second part of the split surfactant is soluble in the dispersed phase, e.g., aqueous phase. The first of the surfactant parts contains a reactive moiety and the second of the surfactant parts contains a complementary reactive moiety. Upon partition formation, the continuous and dispersed phase surfactant parts may interact and chemically react forming the full surfactant. A split surfactant can have three or more parts, one or more of each is soluble in the continuous phase while one or more is also soluble in the dispersed, e.g., aqueous phase. Each part of the split surfactant contains a reactive moiety such that the close proximity of the parts results in formation of the full surfactant. In certain embodiments, formation of the full surfactant also contains functional moieties, i.e., linkage moieties, for cross-linking using any of the above cross-linking methods. The formation of the full surfactant activates, or allows activation of, the cross-linking, resulting in a network of full formed surfactants linked to each other at the partition interface.

### Cross-Link Characteristics

The integrity and strength of the cross-linked network at the partition interface is dependent on the physical parameters of the cross-linking agents attached to the surfactants. These include the (1) strength of the interaction between the one or more cross-linking agents, (2) relative degree of cross-linking, i.e. the number of surfactant-surfactant interactions that can range from 0% to 100% surfactant networking, (3) the length of the spacer arms between the surfactant and the cross-linking agent, e.g., the length of the cross-link, and (4) the number of cross-linking agents, e.g., linkage moieties, attached to each surfactant and the polydispersity in the number of cross-linking agents, e.g., linkage moieties, attached to each surfactant.

Interaction strength: The relative strength of the interaction between two cross-linked surfactants will determine the overall strength of the network at the partition interface. Ionic lattice energies typically fall in the range of 600-4000 kJ/mol (some even higher), covalent bond dissociation energies are typically between 150-400 kJ/mol for single bonds, hydrogen bond dissociation energies typically range from 4-21 kJ/mol, van der Walls dissociation energies range from 1-4 kJ/mol. The higher bond dissociation energies for ionic networks is due to the formation of ionic lattices. Bond dissociation energies for biologically relevant crosslinking agents include: H—H: 436 kJ/mol, H—C: 415 kJ/mol, H—N: 390 kJ/mol, H—O: 464 kJ/mol, H—F: 569 kJ/mol, H—Si: 395 kJ/mol, H—P: 320 kJ/mol, H—S: 340 kJ/mol, H—Cl: 432 kJ/mol, H—Br: 370 kJ/mol, H—I: 295 kJ/mol, C—C: 345 kJ/mol, C═C: 611 kJ/mol, C≡C: 837 kJ/mol, C—N: 290 kJ/mol, C═N: 615 kJ/mol, CEN: 891 kJ/mol, C—O: 350 kJ/mol, C═O: 741 kJ/mol, CEO: 1080 kJ/mol, C—F: 439 kJ/mol, C—Si: 360 kJ/mol, C—P: 265 kJ/mol, C—S: 260 kJ/mol, C—Cl: 330 kJ/mol, C—Br: 275 kJ/mol, C—I: 240 kJ/mol, N—N: 160 kJ/mol, N═N: 418 kJ/mol, N≡N: 946 kJ/mol, N—O: 200 kJ/mol, N—F: 270 kJ/mol, N—P: 210 kJ/mol, N—Cl: 200 kJ/mol, N—Br: 245 kJ/mol, O—O: 140 kJ/mol, O═O: 498 kJ/mol, O—F: 160 kJ/mol, O—Si: 370 kJ/mol, O—P: 350 kJ/mol, O—Cl: 205 kJ/mol, O—I: 200 kJ/mol, F—F: 160 kJ/mol, F—Si: 540 kJ/mol, F—P: 489 kJ/mol, F—S: 285 kJ/mol, F—Cl: 255 kJ/mol, F—Br: 235 kJ/mol, Si—Si: 230 kJ/mol, Si—P: 215 kJ/mol, Si—S: 225 kJ/mol, Si—Cl: 359 kJ/mol, Si—Br: 290 kJ/mol, Si—I: 215 kJ/mol, P-P: 215 kJ/mol, P—S: 230 kJ/mol, P—Cl: 330 kJ/mol, P—Br: 270 kJ/mol, P—I: 215 kJ/mol, S—S: 215 kJ/mol, S—Cl: 250 kJ/mol, S—Br: 215 kJ/mol, Cl—Cl: 243 kJ/mol, Cl—Br: 220 kJ/mol, Cl—I: 210 kJ/mol, Br—Br: 190 kJ/mol, Br—I: 180 kJ/mol, I-I: 150 kJ/mol, Avidin-Biotin: 9-100 kJ/mol. Therefore, cross-linking agents that form bonds with higher dissociation energies will result in a more structurally sound network at the partition interface. In certain embodiments, the strength of the cross-links of a cross-linked surfactant network as provided herein is 1-4000, 1-1000, 1-500, 20-4000, 20-1000, 20-500, 50-4000, 50-1000, 50-500

Degree of cross-linking: The degree of cross-linking is an important factor of network strength at the partition interface. The degree of cross-linking may take any value between 0 and 100%, with network strength generally increasing with increasing degree of cross-linking. The degree of cross-linking may be expressed in any suitable manner. For example, in a system of surfactant molecules there will be a maximum possible number of cross-links (e.g., if surfactant molecules have an average of 4 linkage moieties that can each form only one cross-link, then each surfactant molecule can, on average, make a maximum of 4 cross-links) and a minimum number (zero), and degree of cross-linking can be expressed as a percentage of maximum (e.g., in the above example, if each surfactant molecule makes, on average, 3 cross-links out of a possible maximum of 4, then degree of cross-linking is 75%). However, in general the percentage of neighbors of surfactant molecules that are cross-linked is a more useful comparison, as described elsewhere herein, i.e., if no neighboring surfactants are cross-linked, the degree of cross-linking is 0%. If all neighbors of every surfactant is cross-linked, the degree of cross-linking is 100%. Degree of cross-linking may be determined in any suitable manner, for example, by determining the average number of linkage moieties per surfactant through stoichiometric calculations based on the reagent concentrations used in attaching moieties to surfactant molecules and likely completeness of reaction, determining the average number of neighbors of each surfactant at the partition interface, and determining the stoichiometric ratio of intermediate linkage moieties to linkage moieties, if applicable, as well as the efficiency of the linkage interactions between moieties. For example, surfactant may be prepared with biotin attached using a 10-fold excess of biotin to surfactant and a reaction efficiency of 60%, so that the average number of biotins per surfactant is 6. The surfactant molecules may form a network where the average number of neighbors is 5, and streptavidin may be present in a 10× excess to the average number of surfactants in the partitions. Because streptavidin-biotin interactions are very efficient, it can be assumed that binding efficiency is 100%, and, since both linkage moieties and intermediate linkage moieties are present in excess, degree of cross-linking would be calculated to be 100% in this case. FRAP measurements, as described elsewhere herein, may also be used. In certain embodiments, the degree of cross-linking is 1-100, 2-100, 5-100, 10-100, 20-100, 30-100, 40-100, 50-100, 60-100, 70-100, 80-100, 90-100, 95-100, 98-100, or 99-100%, or at least 5, 10, 15, 20, 30, 35, 40, 45, 50, 55, 60, 65, 70, 75, 80, 85, 90, 95, or 100%, or any range therebetween

Cross-link length: Since the spacing arm will dictate the physical distance between the surfactant molecule and its cross-linking agent, e.g., overall, the length of the linkage moiety, as well as the distance between two cross-linked surfactants, length can be an important variable in cross-linking efficiency and strength. Too short of spacing arms may sterically reduce access of a cross-linking agent to the reactive moiety on the surfactant to be cross-linked, may reduce the extent of surfactant-surfactant interactions by not allowing the cross-linking agents of each surfactant to be in close enough proximity for a reaction to occur, or may allow two surfactants to cross-link while restricting the ability to cross-link to three or more surfactants limiting the degree of surfactant networking at the droplet interface. Too long of cross-linking agents, while the may allow for a high degree of surfactant networking, may result in a loose and/or floppy network that does not improve the stability of the partition interface. In some embodiments, the length of the spacer arm is tailored for the size range of droplets that are stabilized for a particular application. Generally, the average cross-link length can be calculated by adding the length of linkage moieties on each surfactant molecule and subtracting any length of overlap (e.g., in the case of complementary oligonucleotides, the length of complementary sequences that will anneal) and adding the length of any intermediate linkage moieties used. The length of the linkage moiety can be any suitable length, such as 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the head group of the surfactant, or 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the tail group of the surfactant, or of the head group of the surfactant. In certain embodiments, the cross-linker is 0.1-100 nm, 0.1-50 nm, 0.1-30 nm, 0.1-20 nm, 0.1-10 nm, 0.1-5 nm, 0.1-3 nm, 0.1-1 nm, 0.1-0.5 nm, 1-100 nm, 1-50 nm, 1-30 nm, 1-20 nm, 1-10 nm, 1-5 nm, or 1-3 nm in length.

Linkage moiety number per surfactant: The number of cross-linking agents per surfactant molecule, e.g. number of linkage moieties per surfactant, is one factor dictating the degree of cross-linking attained in the surfactant network. For example, a homogenous surfactant bearing only one cross-linking agent, e.g., linkage moiety, per surfactant will be able to form a maximum of 1 interaction with another surfactant molecule whereas surfactants with two or more cross-linking agents, e.g., linkage moieties, may form cross-links with 1 or more additional surfactants. For example, surfactant molecules modified with a single biotin moiety have the ability to link to one streptavidin molecule, and due to the tetrameric nature of the streptavidin protein, a single surfactant may then be linked to up to 3 additional surfactant molecules. Surfactant molecules modified with two or more biotin moieties may interact with one or more streptavidin proteins each of which can interact with up to 3 similar or different surfactant molecules. It will be appreciated that in order to achieve a high degree of cross-linking, the number of linkage moieties per surfactant should approach, equal, or exceed the average number of neighbors for the surfactant at the partition interface. For example, if the average number of neighbors is five, then, in order to achieve 100% degree of cross-linking, each surfactant should have at least 5 linkage moieties. The number of linkage moieties per surfactant can be calculated from the stoichiometric ratio of linkage moieties to surfactant molecules during attachment of the linkage moieties, and the efficiency of the attachment conditions. For example, if a 10-fold excess of linkage moieties to surfactant molecules is used and the efficiency of attachment is 70%, the average number of linkage moieties per surfactant molecule can be taken to be 7. The average number of linkage moieties per surfactant molecule can be any suitable number, such as 2-20, 2-15, 2-12, 2-10, 2-8, 2-6, 2-4, 3-20, 3-15, 3-12, 3-10, 3-8, 3-6, 4-20, 4-15, 4-12, 4-10, 4-8, 4-6, 5-20, 5-15, 5-12, 5-10, 5-8, or 5-7; or about 2, 3, 4, 5, 6, 7, 8, 9, 10, 11, 12, 13, 14, 15, 16, 17, 18, 19, or 20, or any range therebetween.

### Stability of Cross-Linked Surfactant Networks

In all embodiments, the resulting cross-linked surfactant, e.g., fluorosurfactant results in a stabilization of the droplet interface.

Depending on the specific chemistry utilized for droplet interface stabilization, the generated shell, e.g., cross-linked surfactant network, may or may not reduce small and/or large molecule diffusion into between the dispersed, e.g., aqueous phase and the continuous, e.g., oil phase (e.g. by direct diffusion and/or by reverse micelle formation), promote gas exchange between the two phases, or promote molecular adsorption to the interface. Alternatively or additionally, coalescence of partitions may be reduced or eliminated.

This may be manifested in a number of ways including but not limited to the reduction in surfactant retention at the partition interface by the disruption of reverse micelle formation, the generation of a highly hydrophilic, e.g., charged partition interface, the generation of a highly hydrophobic droplet interface, some combination thereof, and/or the generation of a cross-linked surfactant network with a defined porosity. The use of networks of cross-linked surfactant molecules at the partition interface may allow the use of various moieties, especially in the partition interior, that might otherwise not be able to be used, or that can be used only in low concentrations. These moieties include lysate constituents from tissue, blood, plant material, microbial cells; pharmaceuticals and their precursors or derivatives; toxicological agents; growth medium constituents; bioproduction substrates, intermediates, or products; metabolic intermediates; lipids; inducing agents; antibiotics; detergents; crowding agents; enzyme substrates, products, or inhibitors, reporter molecules, or any combination thereof.

Depending on the specific surfactant chemistry, surfactants are often soluble in either the continuous phase or dispersed phase in a micellular state. Upon formation of the partition interface, the micelles dissociate, and the surfactant molecules embed themselves in the partition interface. Since the surfactant are amphiphilic and have at least two groups of opposed solubility, the tail and head groups associate with their respective solvents. The solubility equilibrium reached between the micelle state and the interface is a dynamic process that exists in the constant exchange of surfactant molecules in the partition interface and solvent dissolved surfactants in a micelle state. This exchange of surfactants between the partition interface and micelles is called reverse micelle formation. During reverse micelle formation, dispersed phase soluble molecules may be encapsulated in the micelle effectively solubilizing it in the continuous phase. These ‘loaded’ micelles might remain permanently in the continuous phase, reform in the partition interface of the same partition from which it was generated, or reform in a separate partition. The transit of micelles from partition to partition carrying dispersed phase molecules enables the transit of molecules between partitions even though those molecules are not soluble in the continuous phase outright. This phenomenon may be counteracted by surfactant cross-linking at the partition interface. Since the surfactant molecules are physically linked at the partition interface, reverse micelle formation is reduced effectively or even eliminated or substantially eliminated, reducing or eliminating or substantially eliminating transit of dispersed phase molecules between droplets using this mechanism. The extent of reduction is dependent on the extent of surfactant cross-linking.

Alternatively, modulation of the chemistry at the partition interface will selectively reduce transit of molecules with non-complementary chemistries across the partition interface. For example, the generation of a highly hydrophilic, e.g., charged partition interface will limit the transit of hydrophobic molecules across the interface and in general also limit transit of moieties with the same charge or partial charge and potentially increase transit of moieties with opposite charge or partial charge. Alternatively, the generation of a highly hydrophobic interface will limit the transit of hydrophilic molecules across the interface.

Alternatively, the precise generation of cross-linking surfactant networks with defined porosities at the droplet interface will selectively reduce transit of molecules based on size across the partition interface. For example, nominal porosities of 1 kD in the surfactant network will selectively limit molecular transit of molecules greater than 1 kD, while nominal porosities of 10 kD in the surfactant network will selectively limit molecular transit of molecules greater than 10 kD.

Surfactant, for example, fluorosurfactant, cross-linking chemistries that demonstrate reduced small molecule diffusion, high gas diffusion, and mitigated molecular adsorption to the interface can demonstrate high utility in many applications including but not limited to cell, protein, and nucleic acid assays.

Surfactant, for example, fluorosurfactant, cross-linking chemistries that demonstrate reduced small molecule diffusion, low gas diffusion, and mitigated molecular adsorption will demonstrate high utility in many applications, including but not limited to cell, protein, and nucleic acid assays. Any suitable surfactant that is amenable to cross-linking may be used in the methods and compositions provided herein. Exemplary surfactants include nonionic surfactants, anionic surfactants, cationic surfactants, and zwitterionic surfactants; in certain embodiments, the surfactant comprises one or more fluorosurfactants. Non-limiting classes of anionic surfactants include linear alkylbenzene sulfonates (LAS), alcohol ether sulfates (AES), secondary alkane sulfonates (SAS) and alcohol sulfates (AS). Examples of anionic surfactant groups include sulfonic acid salts, alcohol sulfates, alkylbenzene sulfonates, phosphoric acid esters, and carboxylic acid salts. In certain embodiments, the surfactant is an anionic fluorosurfactant, such as perfluoronanoate or perfluorooctonate, or any other suitable anionic fluorosurfactant. Exemplary cationic surfactants include primary, secondary, or tertiary amines; in certain embodiments, a cationic surfactant is a quaternary amine such as CTAB, CPC, BAC, BZT, or DODAB. In zwitterionic surfactants, the cationic portion may be based on primary, secondary, or tertiary amines or quaternary ammonium cations. The anionic part can include sulfonates, carboxylates, phosphates or the like; in certain embodiments a zwitterionic surfactant comprises a phosphate anion with an amine or ammonium, such as phospholipids, for example phosphatidylserine, phosphatidylethanolamine, phosphatidylcholine, and sphingomyelins. Nonionic surfactants can have, e.g., covalently bonded non-ionic oxygen groups in their hydrophilic head, attached to a hydrophobic tail group.

Exemplary fluorosurfactants comprise an oligoethylene glycol, TRIS, or polyethylene glycol moiety. In certain embodiments, fluorosurfactants comprise a fluorocarbon and/or chlorofluorocarbon moiety. In some embodiments, fluorosurfactants have head and tail moieties linked by ether, amide, or carbamide bonds. In a certain embodiments, fluorosurfactants have a polyethylene glycol moiety linked to a fluorocarbon moiety through a carbamide, ether, or amide bond. Fluorinated surfactants include but are not limited to Picosurf-1, Ran FS-008, FC-4430, FC-4432, FC-4434. In certain cases, the fluorosurfactant can comprise a polyethylene moiety linked to a fluorocarbon moiety with a carbamide, amide, or ether bond. In certain embodiments in which biotin is used as a linkage moiety, an exemplary fluorosurfactant, including biotin, is FS-Biotin from Ran Biotechnologies. See, e.g., US Patent Application Publication No. 20180112036.

Any suitable continuous phase may be used in methods and compositions provided herein. In certain embodiments, the continuous phase comprise an oil; in certain embodiments the oil is a fluorinated oil. Exemplary oils include those described elsewhere herein. Exemplary fluorinated oils include those described elsewhere herein.

Alternatively or additionally, cross-linked surfactant networks at partition interfaces may reduce or eliminate coalescence of partitions.

Assessment of stability of cross-linked surfactant networks. In general, improved stability will decrease the movement of components from one partition to another through diffusion, reverse micelle formation, and/or coalescence, and/or can improve efficiency and/or accuracy of processes carried out with the emulsion. Coalescence also reduces the total number of partitions. In certain embodiments, methods and compositions provided herein for formation of cross-linked surfactant networks can increase stability of an emulsion compared to the same emulsion without cross-linking, e.g., prepared with surfactant molecules without attached linkage moieties and/or without necessary components and/or conditions for completion of linkage processes.

Stability can be assessed in any suitable manner.

In certain embodiments, stability is evaluated by the movement of detectable species, such as dye molecules, from partitions containing the molecules to partitions that do not contain the molecules, after incubation under defined conditions and time. The species may be selected to have a certain size, hydrophilicity, e.g., charge, hydrophobicity, or combination thereof. For example, a small water-soluble dye may be provided in a population of partitions, and assessment of its subsequent transfer to a population of partitions that do not contain the dye after mixing the two populations may be used. Exemplary dyes include rodamine CG, which typically has a very fast exchange rate, on the order of minutes; resorufin, with an intermediate exchange rate, on the order of hours; fluorescein, with a slow exchange rate, on the order of days.

Exemplary conditions can be production of two populations of partitions under identical conditions, one containing the desired dye at an appropriate concentration, e.g., 2 uM, and the other not containing the dye, then mixing equal amounts of the two populations, for example in an Eppendorf tube. Any suitable method may be used to produce the two populations, such as the method described in Guner et al., Controlling molecular transport in minimal emulsions, Nat Comm DOI 10.1038/natcomms10392, 2016. In certain embodiments, a Dropworks PCR system is used where the droplet generator is unhooked from the rest of the system and appropriate dyes, surfactant, dispersed phase, and continuous phase are fed into the droplet generator e.g., from a 20 uL sample split into, e.g., 20,000, 25,000; 30,000; 35,000; 40,000; 45,000; or 50,000 droplets or any range therebetween, and separate volumes of empty (no dye) and dye-containing droplets are formed per the same conditions as for a PCR assay, then the two populations are combined. The combined populations are then incubated under suitable conditions; it can desirable to incubate the populations at elevated temperature, e.g., at about 50, 60, 70, 80, 85, 86, 87, 88, 89, 90, 91, 92, 93, 94, 95, 96, 97, 98, or 99° C., or any range therebetween. An appropriate duration for the incubation is chosen depending on the dye used. For a dye with a fast exchange rate, e.g., rodamine CG, an incubation time of minutes can be appropriate, e.g., 1, 2, 3, 4, 5, 6, 7, 8, 9, 10, 12, 15, 17, 20, 15, 30, 40, 50, 60, 70, 80, 90, 100, 110, 120 minutes. For a dye with an intermediate exchange rate, e.g., resorufin, longer incubation times may be appropriate (depending on temperature), e.g., 1, 2, 3, 4, 5, 6, 7, 8, 9, 10, 12, 15, 17, 20, 15, 30, 40, 50, 60, 70, 80, 90, 100, 110, 120 minutes, or 2.5, 3, 3.5, 4, 4.5, 5, 5.5, or 6 hours. For a dye with a long exchange rate, e.g., fluorescein, even longer incubation times may be appropriate (again depending on temperature), e.g., 0.5, 1, 1.5, 2.0, 2.5, 3, 3.5, 4, 4.5, 5, 5.5, 6, 7, 8, 9, 10, 11, 12, 15, 20, 24, 30, 36, 42, 48, 72, or 96 hours. At an appropriate time point, the population can be imaged by microscopy and fluorescence intensity of the dye-free droplets can be assessed using, e.g. the line profile tool of Leics software. Mean fluorescence value of randomly selected portions of the non-dye population, e.g., 10, 20, 30, 50, or 100 randomly selected partitions, of the time 0 populations can be assumed to be background and compared to the fluorescence values at one or more of days 1, 2, or 3. The test can be carried out for partitions that are cross-linked vs. partitions that are not. If the fluorescence value for the non-cross-linked partitions at a given time point is taken to be 100%, stability of the cross-linked partitions can be expressed as the decrease in diffusion compared to non-cross-linked, as reflected in decrease in fluorescence of the cross-linked samples, e.g., if the fluorescence of non-cross-linked partitions is an average of 100 units and that of cross-linked partitions is 40 units, the decrease in diffusion is 60%. Increase in stability can be expressed as this % decrease in diffusion, for a specific dye molecule. In certain embodiments, methods and compositions provided herein can result in an increase in stability of droplets as determined by a dye diffusion assay such as described herein of at least 1, 5, 10, 15, 20, 25, 30, 35, 40, 50, 60, 70, 80, 90, 95, or 99%, or any range therebetween.

In certain embodiments, stability of partitions may be assessed through measurements of coalescence. Any suitable method may be used to produce droplets for such an assay, such as the method described in Guner et al., Controlling molecular transport in minimal emulsions, Nat Comm DOI 10.1038/natcomms10392, 2016. In certain embodiments, a Dropworks PCR system is used where the droplet generator is unhooked from the rest of the system and appropriate surfactant, dispersed phase, and continuous phase are fed into the droplet generator and a volume of droplets is obtained, e.g., from a 20 uL sample split into, e.g., 20,000, 25,000; 30,000; 35,000; 40,000; 45,000; or 50,000 droplets or any range therebetween. If a monodisperse population of partitions is produced, e.g., a population where most or substantially all of the partitions are within a relatively small size range, e.g., range of diameters for droplets, a relatively straightforward test of coalescence is to visualize the emulsion at various timepoints after subjecting the emulsion to some standard conditions, and determine the relative amount of coalescence by determining the relative percentage of partitions whose volume has increased to at least 2× the initial volume of the partitions. Emulsions can be produced as described for dye measurements, except that it is not necessary to combine two populations, so long as droplets are sufficiently visible so that size (diameter) can be determined or relative diameter can be determined. Thus, at time 0 the percentage of fused partitions may be low, e.g., 0 or close to 0, then at subsequent times it may increase to 10, 20, 50, or higher than 50%. It will be appreciated that each fused partition represents at least 2 unfused partitions, but to simplify, percentages can be measured at each timepoint by counting fused partitions in the field, counting total number of partitions, dividing the former by the latter, and multiplying by 100. As with other tests, performance of cross-linked partitions can be compared to the same partitions without cross-linking. In some cases this can be accomplished merely by removing the presence of a cross-linking agent (e.g., streptavidin) or a cross-linking stimulus (e.g., light; in other cases more extensive modification may be necessary but in general it is desirable to modify the non cross-linked partitions as little as possible compared to the cross-linked partitions. An exemplary test of coalescence is to form cross-linked and non-cross-linked partitions in separate populations, preferably but not necessarily at the same time, image partitions at time 0 to determine initial coalescence, then hold the partitions under conditions that favor coalescence, for example, at an elevated temperature such as 40, 50, 60, 70, 80, or 90° C., and determine coalescence at one or more time points by visual, e.g., microscopic, examination, such as at 10, 20, 30, 40, 50 and/or 60 minutes, and/or 1, 1.5, 2, 2.5, 3, 3.5, 4, 4.5, 5, 5.5, 6, 6.5, 7, 7.5, 8, 9, and/or 10 hours, and express coalescence as a percentage. In certain cases agitation may be added so long as it is of the same degree between the two populations. Stability of cross-linked partitions can be expressed as the decrease in coalescence compared to non-cross-linked. For example, if both sets of emulsions start out at 0 coalescence, and at 1 hr the non-cross-linked emulsion exhibits 80% coalescence, e.g., as defined above, and the cross-linked emulsion exhibits 20% coalescence, the increased stability of the cross-linked emulsion compared to non-cross-linked, can be expressed as 75% (decrease in coalescence under the specified conditions). In certain embodiments, methods and compositions provided herein can result in an increase in stability of droplets as determined by a coalescence assay such as described in this paragraph of at least 1, 5, 10, 15, 20, 25, 30, 35, 40, 50, 60, 70, 80, 90, 95, or 99%, or any range therebetween.

Another assay for coalescence that can be used is to prepare a population of droplets with surfactant cross-linking and another population without surfactant cross-linking, and run the two populations through the process system of interest, e.g., a PCR system, such as the Dropworks PCR system, e.g., under conditions that droplets would be subjected to in a PCR assay. If a detector is used that can estimate droplet volume, the initial number of droplets produced at, e.g., a droplet generator can be estimated by dividing sample volume by average droplet volume, e.g., a 20 uL sample that produced droplets with an average volume of 0.57 nL can be assumed to have produced ˜35,000 droplets initially. The number of droplets that pass through the detector can be counted, and the difference between this number and the initial number represents coalesced droplets; alternatively or additionally, volumes of droplets measured at the detector can be used to determine which droplets represent coalesced droplets, where droplets with 2× expected volume or greater are considered to be coalesced. From these numbers, a percentage of coalescence can be determined for cross-linked vs non-cross-linked and compared. In this assay, reduction in coalescence expressed as a percent can correspond to increase in stability. In certain embodiments, methods and compositions provided herein can result in an increase in stability of droplets as determined by a coalescence assay such as described in this paragraph of at least 1, 5, 10, 15, 20, 25, 30, 35, 40, 50, 60, 70, 80, 90, 95, or 99%, or any range therebetween.

Other tests of partition stability may be used, in particular tests that mimic or reproduce conditions under which the partitions may be used. For example, in a digital PCR system, which under normal operation produces, e.g., tens of thousands of droplets from a sample containing nucleic acids of interest, where the concentrations of materials and conditions of droplet formation are such that the majority of droplets, e.g., at least 90, 95, 99, or 99.9% of the droplets contain one or no nucleic acids of interest, controlled runs in which the only difference is surfactant network cross-linking in one or a plurality of runs and no cross-linking in one or a plurality of other runs, may be compared. For example, known amounts of a first test oligonucleotide and a second test oligonucleotide may be combined in a test sample The molar amount of the two test oligonucleotides may be the same or substantially the same or it may be different; in certain embodiments, the amount of each is the same or substantially the same. The sample also contains primers, buffer, enzymes, and any other necessary components for PCR; a first fluorescent label is present that is specific for the first oligonucleotide's amplification product, and a second fluorescent label is present that is specific for the second oligonucleotide's amplification product; the two labels are different and fluoresce at detectably distinct wavelengths.

The test sample is transported to a droplet generation system, such as the Dropworks system described for producing droplets for dye transit and coalescence tests; however, in this case the system is fluidly connected to the bulk of the PCR system, e.g., a thermal cycler and a detector. For a first aliquot of the test sample, droplets are generated by flowing test sample (an aqueous dispersed phase) into a first inlet in the droplet generator, and continuous phase (e.g., oil such as a fluorinated oil) comprising surfactant that has not been treated to produce cross-linked surfactant networks into a second inlet of the droplet generator; inside the droplet generator the first and second inlets intersect and droplets of sample in the continuous phase are formed. It will be appreciated that in embodiments in which dispersed phase comprises components that initiate or promote cross-linking to produce cross-linked surfactant networks, these components are not included; similarly, if droplets are exposed to one or more conditions that initiate or promote cross-linking, those conditions are not present. The droplets are formed in a monodisperse emulsion, e.g., from a 20 uL sample split into, e.g., 20,000, 25,000; 30,000; 35,000; 40,000; 45,000; or 50,000 droplets or any range therebetween, and the concentration of sample, flow rates, and other relevant factors are such that the majority of droplets, e.g., at least 90, 95, 99, 99.5, 99.9, 99.95, or 99.99% of the droplets contain only one of the first test oligonucleotide, only one of the second test oligonucleotide, or none of either. The first and second test oligonucleotides can be any suitable length, such as about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 57, 60, 62, 65, 67, 70, 75, 80, 85, 90, 95, 100, 110, 120, 150, 170, 200, or 250 nucleotides, or any range therebetween, and may be the same or different lengths, though generally the lengths should be similar or the same. Any suitable sequence may be used for the oligonucleotides so long as the two sequences are distinguishable from each other when amplified and detected. The bolus of droplets is then sent through the PCR system, e.g., to a thermal cycler then a detector. In some cases, the diameter of conduit increases at the thermal cycler, slowing flow rate. The detector may be any detector that is capable of detecting single droplets and distinguishing the two different fluorescent labels used. Preferably, the detector is also capable of determining volume of droplets to determine what percentage of droplets coalesced between the droplet generator and the detector. The total number of droplets that contain no amplified oligonucleotide, that contain only amplified first oligonucleotide, that contain only amplified second oligonucleotide, and that contain both amplified first and second oligonucleotides is determined. In detectors that can detect droplet volume or a quantity related to volume, the number of coalesced droplets, which will have a volume of nX, where n is the number of droplets that have coalesced and X is the volume of a non-coalesced droplet, can be determined. The number of these droplets that contain no oligonucleotide or amplification product of only the first or only the second oligonucleotide can be added to the number of droplets that contain both amplified first and second oligonucleotides; it will be appreciated that the number of coalesced droplets that contain both has already been counted in the previous step and so shouldn't be added again. The number of the latter divided by total droplet number and multiplied by 100 is taken as the percentage of droplets subject to transit, i.e., for the vast majority of such droplets, both oligonucleotides are present or amplified because one of them transited from its original droplet to the detected droplet. The same test is run for the same sample except this time surfactant in continuous phase and/or components in dispersed phase and/or conditions droplets are exposed to during and/or after formation are such as to produce the cross-linked surfactant network to be tested. The same calculation is performed to determine a second percentage of droplets subject to transit. The first percentage (no cross-linking) can be divided by the second number (cross-linking) to give a quantitative indication of the effectiveness of the cross-linking. For example, in a system that uses biotin as a linkage moiety attached to surfactant molecules and streptavidin as an intermediate moiety to cross-link biotins, a first run comprising oligo 1 and oligo 2 is run, where the surfactant in continuous phase does not have biotin moieties attached and the dispersed phase does not contain streptavidin. 20,000 droplets are produced at the droplet generator and run through the thermal cycler and to the detector. The detector detects 18,000 droplets with no oligo; 800 droplets with only oligo 1; 800 droplets with only oligo 2; and 400 droplets with both oligo 1 and 2. In addition, the detector detects that 150 of the droplets were at least double the volume of a normal droplet, of which 50 contain both oligo 1 and oligo 2. The percentage of transit “events” is (400+(150−50))/20,000×100, or 500/20,000×100=2.5%. A second run comprising oligo 1 and oligo 2 is then run, where the surfactant in continuous phase does have biotin moieties attached and the dispersed phase does contain streptavidin. 20,000 droplets are produced, and the detector detects 18,000 droplets with no oligo, 990 droplets with only oligo 1, 990 droplets with only oligo 2, and 20 droplets with both oligo 1 and 2. In addition, the detector detects that 15 of the droplets were at least double the volume of a normal droplet, of which 5 contain both oligo 1 and oligo 2. The percentage of transit “events” is (20+(15−5))/20,000×100, or 25/20,000×100=0.125%. In this case, it can be taken that transit between droplets has been improved by 95%. It will be appreciated that this number does not necessarily reflect all transit processes that occur (e.g., oligo 1 transits to empty droplet, or oligo 1 transits to another droplet with oligo 1, etc.); however, it can serve as an appropriate indication of improvement in keeping droplet contents discrete in the PCR system. In certain embodiments, the calculation can be refined to account for transit of oligo that results in 2 of the same oligo in a single droplet provided that the detector has sufficient ability to detect a difference in fluorescence intensity between such droplets. In certain embodiments, methods and compositions provided herein can result in an increase in stability of droplets as determined by a coalescence assay such as described in this paragraph of at least 1, 5, 10, 15, 20, 25, 30, 35, 40, 50, 60, 70, 80, 90, 95, or 99%, or any range therebetween.

Thus, in certain embodiments, provided herein is a method of increasing partition stability in an emulsion, wherein the emulsion comprises a plurality of dispersed phase partitions in a continuous phase and wherein the partitions comprise surfactant moieties at interfaces between dispersed phase in partitions and continuous phase, comprising producing a cross-linked surfactant network of the surfactant moieties at the interface, wherein the increase in stability is assessed by a dye diffusion assay, such as an assay described herein, and wherein surfactant network stability, as reflected in a decrease in dye diffusion, is increased by at least 1, 2, 5, 10, 20, 30, 40, 50, 60, 70, 75, 80, 85, 90, 95, 96, 97, 98, 99, 99.5, 99.9, or 100%, or any range of values therebetween, for example, by at least 50%, such as at least 80%, in some cases at least 90%. Any suitable method for cross-linking, such as those described herein, may be used.

In certain embodiments, provided herein is a method of increasing partition stability in an emulsion, wherein the emulsion comprises a plurality of dispersed phase partitions in a continuous phase and wherein the partitions comprise surfactant moieties at interfaces between dispersed phase in partitions and continuous phase, comprising producing a cross-linked surfactant network of the surfactant moieties at the interface, wherein the increase in stability is assessed by a coalescence assay, such as an assay described herein, and wherein surfactant network stability, as reflected in a decrease in coalescence, is increased by at least 1, 2, 5, 10, 20, 30, 40, 50, 60, 70, 75, 80, 85, 90, 95, 96, 97, 98, 99, 99.5, 99.9, or 100%, or any range of values therebetween, for example, by at least 50%, such as at least 80%, in some cases at least 90%. Any suitable method for cross-linking, such as those described herein, may be used.

In certain embodiments, provided herein is a method of increasing partition stability in an emulsion, wherein the emulsion comprises a plurality of dispersed phase partitions in a continuous phase and wherein the partitions comprise surfactant moieties at interfaces between dispersed phase in partitions and continuous phase, comprising producing a cross-linked surfactant network of the surfactant moieties at the interface, wherein the increase in stability is assessed by a PCR assay, such as an assay described herein, and wherein surfactant network stability, as reflected in a decrease in PCR events indicative of cross-talk between partitions, is increased by at least 1, 2, 5, 10, 20, 30, 40, 50, 60, 70, 75, 80, 85, 90, 95, 96, 97, 98, 99, 99.5, 99.9, or 100%, or any range of values therebetween, for example, by at least 50%, such as at least 80%, in some cases at least 90%. Any suitable method for cross-linking, such as those described herein, may be used.

### Methods for Destabilizing Cross-Linked Surfactant Networks

Depending on the specific chemistry used to cross-link the surfactants, a number of different methods may be used to break the droplets (partitions) after use, e.g., when recovery of the droplet contents is needed for downstream analysis. This may be done by any suitable method, e.g., chemically or mechanically.

In one embodiment, a disrupting agent is added to the emulsion to disrupt protein cross-linking agents that results in the subsequent deconstruction of the emulsion. In some embodiments, acids, bases and/or peroxides may also be used for network deconstruction. In a preferred embodiment, chloroform is used for network deconstruction.

In a separate embodiment, cleavable linkers may be used to break the droplets after use. The cleavable linkers can be selected among the group consisting of enzymatically cleavable linkers, nucleophile/base sensitive linkers, reduction sensitive linkers, photocleavable linkers, electrophile/acid sensitive linkers, and oxidation sensitive linkers, for instance as illustrated in Leriche, et al. Bioorg. Med. Chem. 20, 571 (2012). Other examples of cleavable linkers can be found in West et al. Current Drug Discovery Technologies, 2, 123 (2005).

In some embodiments, mechanical methods may be used to break the network at the partition interface including but not limited to freezing, mechanical fracture, sonication, heating, and osmotic shock.

In certain embodiments, a specific cross-linking agent degrading molecule may be utilized including but not limited to nucleases for nucleic acid-based cross-linking agents and/or proteases for protein-based cross-linking agents.

### Aqeuous Additives

Depending on the robustness of the partition interface, surfactant stabilized droplets may remain stable in the presence of one or more additives. Due to the increase in stability generated by the cross-linked surfactant network, partitions with cross-linked surfactant networks may tolerate higher levels and/or different combinations of additives than their non-cross-linked alternatives. In certain embodiments, partitions comprising cross-linked surfactant networks may tolerate at least 10, 20, 30, 40, 50, 70, 100, 150, 200, 500, or 1000%, or any range therebetween, of the amount, e.g., concentration, of one or more additive compared to the same partitions without cross-linked surfactant networks. “Tolerating” the additional additive means, in general, that the partitions with cross-linked surfactant networks and/or the emulsion in which they are contained perform at least as well, or at least 10, 20, 50, 70, 80, 90, 95, 97, 99, 99.5, or 99.9% as well, and in some cases better than, partitions of the same composition except without cross-linked surfactant networks and/or the emulsion in which they are contained, for their intended use. The performance can be evaluated by any suitable means, such as, for an assay, e.g., PCR, using one or more control samples of known concentration and comparing the accuracy and/or precision of results with cross-linked vs. non cross-linked partitions.

The aqueous phase may contain one or more buffering compound. Common examples of biological buffers include but are not limited to tris, phosphate, citrate, and the like.

The aqueous phase may contain one or more salt.

The aqueous phase may contain one or more carbohydrate included in but not limited by the following list: Agar, Agarose, Allose, Amylose, Arabinose, Arabitol, Carboxymethyl cellulose, Cellulose, Chitin, Chitosan, Chondroitin, Cyclodextrin, Dextran, Dextrin, Dextrose, Erlose, Erythritol, Erythrose, fructofuranose, Fructose, Galactomannin, Galactose, Glucan, Glucopyranose, Glucosamine, Glucose, Glycogen, Glycosaminoglycan, Gulose, Heparin, Hexitol, Hexopyranose, Iditol, Inositol, Isomaltitol, Kestose, Lactitol, Lactose, Lectin, Melezitose, Maltitol, Maltodextrin, Maltose, Maltulose, Mannitol, Mannose, Melezitose, Panose, Pectin, Polysucrose, Quercitol, Raffinose, Rhamnose, Ribitol, Ribofuranose, Ribose, Ribulose, Rutinose, Sorbitol, Starch, Sucralose, Sucrose, Tagatose, Talitol, Threitol, Threose, Trehalose, Turanose, Xylanose, Xylitol, Xylose.

The aqueous phase may contain one or more protease inhibitors that may target aspartic, cysteine, metallo-, serine, threonine, and trypsin proteases.

The aqueous phase may contain one or more antimicrobial agent.

The aqueous phase may contain one or more crowding agent included in but not limited by the following list: 1,2-propanediol, Carboxymethyl cellulose, Ethylene glycol, Glycerol, PEG 200, PEG300, PEG 400, PEG 600, PEG 1000, PEG 1300, PEG 1600, PEG 1450, PEG 1500, PEG 2000, PEG 3000, PEG 2050, PEG 3350, PEG 4000, PEG 4600, PEG 6000, PEG 8000, PEG 10000, PEG 12000, PEG 20000, PEG 35000, PEG 40000, PEG 108000, PEG 218000, PEG 510000, PEG 90M, Polysucrose, Polyvinyl alcohol, Polyvinylpyroolidone, Propylene glycol.

The aqueous phase may contain one or more detergent. The detergent may be ionic or non-ionic.

The aqueous phase may contain one or more nucleotide or derivatives of the nucleotides included in but not limited by the following list: 5-Fluoroorotic Acid (5-FOA), Adenine, Adenosine, Adenosine diphosphate, Adenosine monophosphate, Adenosine triphosphate, Cytidine, Cytidine diphosphate, Cytidine monophosphate, Cytidine triphosphate, Cytosine, Deoxyadenosine, Deoxyadenosine diphosphate, Deoxyadenosine monophosphate, Deoxyadenosine triphosphate, Deoxycytidine, Deoxycytidine diphosphate, Deoxycytidine monophosphate, Deoxycytidine triphosphate, Deoxyguanosine, Deoxyguanosine diphosphate, Deoxyguanosine monophosphate, Deoxyguanosine triphosphate, Guanine, Guanosine, Guanosine diphosphate, Guanosine monophosphate, Guanosine triphosphate, Hypoxanthine, Inositol, Thymidine, Thymidine diphosphate, Thymidine monophosphate, Thymidine triphosphate, Thymine, Uracil, Uridine, Uridine diphosphate, Uridine monophosphate, Uridine triphosphate. The aqueous phase may contain one or more synthetic nucleotide derivatives.

The aqueous phase may contain one or more amino acid, derivatives of the amino acids, or peptides derived the amino acids.

The aqueous phase may contain one or more vitamin.

The aqueous phase may contain one or more medium additive for the growth, propagation, or induction, death, or analysis of microbial organisms.

Polymerases useful in the methods described herein are capable of catalyzing the incorporation of nucleotides to extend a 3′ hydroxyl terminus of an oligonucleotide bound to a target nucleic acid molecule. Such polymerases include those capable of amplification and/or strand displacement. The polymerase may bear or lack 5′-3′ exonuclease activity. In other embodiments, a polymerase also has reverse transcriptase activity (e.g., Bst (large fragment), Therminator, Therminator II). Exemplary polymerases include but are not limited to BST (large fragment), DNA polymerase I (E. coli), DNA polymerase I, Large (Klenow) fragment, Klenow fragment (3′-5′ exo-), T4 DNA polymerase, T7 DNA polymerase, Deep VentR. (exo-) DNA Polymerase, Deep VentR DNA Polymerase, DyNAzyme, High-Fidelity DNA Polymerase, Therminator, Therminator II DNA Polymerase, AmpliTherm DNA Polymerase, Taq DNA polymerase, Tth DNA polymerase, Tfl DNA polymerase, Tgo DNA polymerase, SP6 DNA polymerase, Thr DNA polymerase. The following non-limiting examples of Reverse Transcriptases (RT) can be used in the reactions of the present method to improve performance when detecting an RNA sequence: OmniScript, SensiScript, MonsterScript, Transcriptor, HIV RT, SuperScript III, ThermoScript, Thermo-X, ImProm II. The following non-limited examples of RNA polymerases include but are not limited to T3, T7, SP6, E. coli RNA pol, RNA pol II, and mtRNA pol. Nicking enzymes useful in the methods described herein include but are not limited to Nt.BspQI, Nb.BbvCI, Nb.BsmI, Nb.BsrDI, Nb.BtsI, Nt.AlwI, Nt.BbvCI, Nt.BstNBI, Nt.CviPII, Nb.Bpu10I, and Nt.Bpu10I.

Other chemical additives include those resulting in changes to pH or ionic strength. Other biological additives include living or dead organisms or their lysates including but not limited to viruses, bacteria, fungi, archaea, plants, and mammalian cells. MgCl2 and KCl concentrations are important for PCR and in some embodiments can be modulated. Detergents such as like triton-x100 can helpful, as well as high protein concentrations.

### Compositions and Methods

In certain embodiments, provided herein is a composition comprising a plurality of first surfactant molecules that have been modified to cross-link with second surfactant molecules under suitable conditions. The surfactant molecules have a tail portion and a head portion. Modification can take the form of a linkage moiety, e.g., a plurality of linkage moieties, attached to the first and/or second surfactant molecules, where the linkage moieties are configured to form cross-link bonds, either with themselves (e.g., first and second linkage moieties are the same) or with another linkage moiety (e.g. first and second linkage moieties are different), or to an intermediate linkage moiety (e.g., that links to one or both of first and second linkage moieties) under suitable conditions. In certain embodiments, the first and second surfactant molecules comprise the same surfactant type, e.g., are structurally the same. In certain embodiments, the first and second surfactant molecules comprise different surfactant types, e.g., are structurally different. First and/or second surfactant molecules of the composition may be attached to any suitable average number of linkage moieties, e.g., 1-20, 1-15, 1-10, 2-20, 2-15, 2-10, 2-8, 2-6, 3-20, 3-15, 3-10, 3-8, 3-5, 4-20, 4-15, 4-10, 4-8, 5-20, 5-15, 5-10, 5-9, 6-20, 6-15, or 6-10 linkage moieties, for example 2-20, such as 2-10, in some cases 3-10. The linkage moieties can be attached to any suitable part of the surfactant molecules, e.g., the head portion or the tail portion by any suitable method of attachment, such as covalent bond or noncovalent bond. The length of the linkage moiety can be any suitable length, such as 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the head group of the surfactant, or 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the tail group of the surfactant. In certain embodiments, the cross-linker is 0.1-100 nm, 0.1-50 nm, 0.1-30 nm, 0.1-20 nm, 0.1-10 nm, 0.1-5 nm, 0.1-3 nm, 0.1-1 nm, 0.1-0.5 nm, 1-100 nm, 1-50 nm, 1-30 nm, 1-20 nm, 1-10 nm, 1-5 nm, or 1-3 nm in length. The surfactant can be any suitable surfactant, such as a nonionic, cationic, anionic, or zwitterionic surfactant. In certain embodiments the surfactant comprises a fluorosurfactant. the fluorosurfactant comprises In certain embodiments the fluorosurfactants have head and tail moieties linked by ether, amide, or carbamide bonds; fluorosurfactants have a polyethylene moiety linked to a fluorocarbon moiety through a carbamide, ether, or amide bond, or a combination thereof. In certain embodiments the fluorosurfactants comprise a polyethylene moiety linked to a fluorocarbon moiety with a carbamide, amide, or ether bond. In certain embodiments the linkage moiety is configured to bind to an intermediate moiety but not to another linkage moiety. In certain embodiments, the linkage moiety is a linkage moiety to form a covalent bond. In certain embodiments, the linkage moiety is a moiety to form a noncovalent bond. Suitable linkage moieties include any as described herein. In certain embodiments, the linkage moiety is biotin. In certain embodiments, the intermediate moiety is a biotin-binding group, such as streptavidin or a streptavidin derivative. The plurality of surfactant molecules may be contained in a continuous phase. The composition may further comprise a continuous phase, such as a hydrophobic continuous phase; in certain embodiments, first surfactant molecules are contained in a first continuous phase, e.g., as micelles; in certain embodiments, second surfactant molecules are contained in a second continuous phase, e.g., as micelles, where the first and second continuous phases can be the same or different. The continuous phase can be an oil, such as a fluorinated oil. Suitable oils and fluorinated oils are as described elsewhere herein. The composition can further comprise a dispersed phase; in certain embodiments, the dispersed phase does not contain the first surfactant molecule; in certain embodiments the dispersed phase does not contain the second surfactant molecule. In certain embodiments, the dispersed phase comprises an intermediate moiety; in cases where the linkage moiety is biotin, the intermediate moiety can be a biotin-binding moiety, such as streptavidin or a streptavidin derivative.

Also provided herein is a composition that contains a plurality of first surfactant molecules comprising a head portion and a tail portion, where the first surfactant molecules have at least one first linkage moiety attached, in a continuous phase. In some cases the surfactant molecules have a plurality of linkage moieties attached, e.g., 2-20, 2-15, 2-10, 2-8, 2-6, 3-20, 3-15, 3-10, 3-8, 3-5, 4-20, 4-15, 4-10, 4-8, 5-20, 5-15, 5-10, 5-9, 6-20, 6-15, or 6-10 linkage moieties, for example 2-20, such as 2-10, in some cases 3-10. In some cases the attachment is covalent. In some cases the attachment is noncovalent. In some cases the linkage moieties are attached to the tail portion of the first surfactant. In some cases the linkage moieties are attached to the head portion of the surfactant molecule. The linkage moieties are configured, under suitable conditions, to link with each other, in some cases covalently, in other cases noncovalently; link with a second linkage moiety different from the first linkage moiety, in some cases covalently, in other cases noncovalently; link with an intermediate linkage moiety, in some cases covalently, in other cases noncovalently; or any combination thereof. In certain embodiments the composition further includes a plurality of the second surfactant molecules, where the second surfactant molecules comprise an average of, e.g., 2-20, 2-15, 2-10, 2-8, 2-6, 3-20, 3-15, 3-10, 3-8, 3-5, 4-20, 4-15, 4-10, 4-8, 5-20, 5-15, 5-10, 5-9, 6-20, 6-15, or 6-10 linkage moieties, for example 2-20, such as 2-10, in some cases 3-10. In some cases the attachment is covalent. In some cases the attachment is noncovalent. In some cases, the first and second linkage moieties are oppositely charged. The composition can further comprise a dispersed phase. The dispersed phase in some cases does not contain the first and/or second surfactant molecule. In some cases the dispersed phase contains the first and/or second surfactant molecules. In some cases the dispersed phase contains one or more components that initiate or promote linkage between the first linkage moieties, between first linkage moieties and intermediate linkage moieties, between first linkage moieties and second linkage moieties, or a combination thereof. In embodiments where the first linkage moiety is, e.g., biotin, the dispersed phase may contain, e.g., a biotin-binding moiety, such as an of the biotin-binding moieties described herein, such as streptavidin or a streptavidin derivative.

In certain embodiments provided herein is an emulsion comprising partitions of a dispersed phase in a continuous phase, where the partitions of the dispersed phase comprise a plurality of surfactant molecules comprising a tail portion and a head portion that are situated at the interface of the partitions with the continuous phase to form a layer of surfactant molecules, and wherein the plurality of surfactant molecules are cross-linked to each other to form a cross-linked network of surfactant molecules. In certain embodiments, the degree of cross-linking is 1-100, 2-100, 5-100, 10-100, 20-100, 30-100, 40-100, 50-100, 60-100, 70-100, 80-100, 90-100, 95-100, 98-100, or 99-100%, or at least 5, 10, 15, 20, 30, 35, 40, 45, 50, 55, 60, 65, 70, 75, 80, 85, 90, 95, or 100%, or any range therebetween, for example 20-100%, such as 40-100%, in some cases 60-100%. The cross-linking can be via cross-links of any suitable length, e.g., 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the head group of the surfactant, or 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the tail group of the surfactant, or the longest dimension of the head group of the surfactant. In certain embodiments, the length of the cross-link is 0.1-100 nm, 0.1-50 nm, 0.1-30 nm, 0.1-20 nm, 0.1-10 nm, 0.1-5 nm, 0.1-3 nm, 0.1-1 nm, 0.1-0.5 nm, 1-100 nm, 1-50 nm, 1-30 nm, 1-20 nm, 1-10 nm, 1-5 nm, or 1-3 nm in length. The surfactant molecules may be cross-linked head-to-head, tail-to-tail, or head-to-tail. In certain embodiments, surfactant molecules comprise linkage moieties that are the same, that is, that have the same structure. In certain embodiments, certain portions of surfactant molecules comprise linkage moieties that are the different from linkage moieties in other portions of surfactant molecules, that is, that have different structures. Additionally or alternatively, all surfactant molecules may be the same, i.e., have the same structure, or surfactant molecules may comprise portions of molecules that have 2, 3, 4, 5, or more than 5 different structures. In certain embodiments, a first portion of the surfactant molecules comprise a first linkage moiety that forms part of the cross-links and a second portion of the surfactant molecules comprise a second linkage moiety, the same as or different from the first linkage moiety, that forms part of the cross-links. The average number of linkage moieties on the surfactant molecules may be 1-20, 1-15, 1-10, 2-20, 2-15, 2-10, 2-8, 2-6, 3-20, 3-15, 3-10, 3-8, 3-5, 4-20, 4-15, 4-10, 4-8, 5-20, 5-15, 5-10, 5-9, 6-20, 6-15, or 6-10 linkage moieties, for example 2-20, such as 2-10, in some cases 3-10. In certain embodiments the continuous phase comprises an oil, such as a fluorinated oil as described herein. In certain embodiments the dispersed phase comprises an aqueous phase. In certain embodiments, surfactant comprises fluorinated surfactant, as described herein. The cross-links may be covalent, non-covalent, or a combination thereof. In certain embodiments, surfactant molecules comprise one or more attached biotin moieties, and cross-linking is via a biotin-binding intermediate moiety, e.g., streptavidin or a streptavidin derivative. In certain embodiments, the cross-linked network of surfactant molecules increases the stability of the partitions in the emulsion, compared to the same emulsion without a cross-linked surfactant network, as measured by a decrease in dye diffusion of at least 20% in a dye diffusion test, such as a dye diffusion test as described herein. In certain embodiments, the cross-linked network of surfactant molecules increases the stability of the partitions in the emulsion, compared to the same emulsion without a cross-linked surfactant network, as measured by a PCR test, such as a PCR test as described herein, by at least 20%. In certain embodiments, the cross-linked network of surfactant molecules increases the stability of the partitions in the emulsion, compared to the same emulsion without a cross-linked surfactant network, as measured by a coalescence assay, such as a coalescence assay described herein, by at least 20%.

In certain embodiments, provided herein are methods of conducting a process in an emulsion of partitions, where the partitions comprise a cross-linked network of surfactant molecules at their surface. Thus, provided herein is a method of conducting a process in an emulsion of partitions of a dispersed phase in a continuous phase, comprising (i) providing the emulsion of partitions of dispersed phase in continuous phase, wherein the partitions of the dispersed phase comprise a plurality of surfactant molecules comprising a tail portion and a head portion that are situated at an interface of the partitions with the continuous phase to form a layer of surfactant molecules, and wherein the plurality of surfactant molecules are cross-linked to each other to form a cross-linked network of surfactant molecules; and (ii) performing the process on the partitions. In certain embodiments, the degree of cross-linking is 1-100, 2-100, 5-100, 10-100, 20-100, 30-100, 40-100, 50-100, 60-100, 70-100, 80-100, 90-100, 95-100, 98-100, or 99-100%, or at least 5, 10, 15, 20, 30, 35, 40, 45, 50, 55, 60, 65, 70, 75, 80, 85, 90, 95, or 100%, or any range therebetween, for example 20-100%, such as 40-100%, in some cases 60-100%. The cross-linking can be via cross-links of any suitable length, e.g., 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the head group of the surfactant, or 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the tail group of the surfactant, or the longest dimension of the head group of the surfactant. In certain embodiments, the length of the cross-link is 0.1-100 nm, 0.1-50 nm, 0.1-30 nm, 0.1-20 nm, 0.1-10 nm, 0.1-5 nm, 0.1-3 nm, 0.1-1 nm, 0.1-0.5 nm, 1-100 nm, 1-50 nm, 1-30 nm, 1-20 nm, 1-10 nm, 1-5 nm, or 1-3 nm in length. The surfactant molecules may be cross-linked head-to-head, tail-to-tail, or head-to-tail. In certain embodiments, surfactant molecules comprise linkage moieties that are the same, that is, that have the same structure. In certain embodiments, certain portions of surfactant molecules comprise linkage moieties that are the different from linkage moieties in other portions of surfactant molecules, that is, that have different structures. Additionally or alternatively, all surfactant molecules may be the same, i.e., have the same structure, or surfactant molecules may comprise portions of molecules that have 2, 3, 4, 5, or more than 5 different structures. In certain embodiments, a first portion of the surfactant molecules comprise a first linkage moiety that forms part of the cross-links and a second portion of the surfactant molecules comprise a second linkage moiety, the same as or different from the first linkage moiety, that forms part of the cross-links. The average number of linkage moieties on the surfactant molecules may be 1-20, 1-15, 1-10, 2-20, 2-15, 2-10, 2-8, 2-6, 3-20, 3-15, 3-10, 3-8, 3-5, 4-20, 4-15, 4-10, 4-8, 5-20, 5-15, 5-10, 5-9, 6-20, 6-15, or 6-10 linkage moieties, for example 2-20, such as 2-10, in some cases 3-10. In certain embodiments the continuous phase comprises an oil, such as a fluorinated oil as described herein. In certain embodiments the dispersed phase comprises an aqueous phase. In certain embodiments, surfactant comprises fluorinated surfactant, as described herein. The cross-links may be covalent, non-covalent, or a combination thereof. In certain embodiments, surfactant molecules comprise one or more attached biotin moieties, and cross-linking is via a biotin-binding intermediate moiety, e.g., streptavidin or a streptavidin derivative. In certain embodiments, the cross-linked network of surfactant molecules increases the stability of the partitions in the emulsion, compared to the same emulsion without a cross-linked surfactant network, as measured by a decrease in dye diffusion of at least 20% in a dye diffusion test, such as a dye diffusion test as described herein. In certain embodiments, the cross-linked network of surfactant molecules increases the stability of the partitions in the emulsion, compared to the same emulsion without a cross-linked surfactant network, as measured by a PCR test, such as a PCR test as described herein, by at least 20%. In certain embodiments, the cross-linked network of surfactant molecules increases the stability of the partitions in the emulsion, compared to the same emulsion without a cross-linked surfactant network, as measured by a coalescence assay, such as a coalescence assay described herein, by at least 20%. The method can further comprise forming the cross-linked surfactant network, e.g., by methods as described herein. In certain embodiments, the cross-linked network of surfactant molecules is formed, or has been formed, by contacting continuous phase comprising a plurality of surfactant molecules that comprise at least one linkage moiety with a dispersed phase under conditions wherein the dispersed phase forms a plurality of partitions in the continuous phase, and providing conditions during and/or after the formation of the partitions that initiate and/or promote formation of cross-links comprising the linkage moieties, wherein the linkage moieties form cross-links from one surfactant molecule to at least one other surfactant molecule, to form a cross-linked network of surfactant molecules. In certain embodiments, the conditions include contact of the linkage moieties with one or more components in the dispersed phase that initiate and/or promote cross-linking reaction. In certain embodiments, the linkage moieties are biotin and the dispersed phase contains a biotin-binding moiety, e.g., streptavidin or a streptavidin derivative. The process may be any suitable process, such as digital PCR, high throughput screening, strain and protein engineering, and cell, protein, and chemical analysis. In certain embodiments, the process is digital PCR. In certain embodiments, the process further comprises breaking open a plurality of the partitions to, e.g., release dispersed phase in the partitions, e.g., breaking open at least 5, 10, 20, 50, 70, 80, 90, 95, or 99% of the partitions or any range therebetween. Methods of breaking the partitions may be as described herein.

In certain embodiments, provided herein are methods for producing an emulsion of partitions of dispersed phase in a continuous phase where the partitions comprise a cross-linked network of surfactant molecules at the interface with the continuous phase. In certain embodiments provided herein is a method for producing an emulsion of partitions of dispersed phase in continuous phase, wherein the partitions of the dispersed phase comprise a plurality of surfactant molecules comprising a tail portion and a head portion that are situated at an interface of the partitions with the continuous phase to form a layer of surfactant molecules, comprising (i) contacting continuous phase with a dispersed phase, wherein either (a) the continuous phase comprises a plurality of surfactant molecules that comprise at least one linkage moiety, or (b) the dispersed phase comprises a plurality of surfactant molecules that comprise at least one linkage moiety, or (c) both (a) and (b) under conditions wherein the dispersed phase forms a plurality of partitions in the continuous phase; and (ii) providing conditions during and/or after the formation of the partitions that initiate and/or promote formation of cross-links between surfactant molecules comprising the linkage moieties, to form a cross-linked network of surfactant molecules. In certain embodiments, the continuous phase contains surfactant molecules with attached linkage moieties and the dispersed phase does not; in certain embodiments, the dispersed phase contains surfactant molecules with attached linkage moieties and the continuous phase does not; in certain embodiments the continuous phase contains a first surfactant with a first attached linkage moiety and the dispersed phase contains a second surfactant with a second attached linkage moiety, where the first and second surfactants can be the same or different and/or the first and second linkage moieties can be the same or different. In certain embodiments, the process is continued until the degree of cross-linking is 1-100, 2-100, 5-100, 10-100, 20-100, 30-100, 40-100, 50-100, 60-100, 70-100, 80-100, 90-100, 95-100, 98-100, or 99-100%, or at least 5, 10, 15, 20, 30, 35, 40, 45, 50, 55, 60, 65, 70, 75, 80, 85, 90, 95, or 100%, or any range therebetween, for example 20-100%, such as 40-100%, in some cases 60-100%. The cross-links formed can be any suitable length, e.g., 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the head group of the surfactant, or 0.1-100×, 0.1-50×, 0.1-30×, 0.1-20×, 0.1-10×, 0.1-5×, 0.1-3×, 0.1-2×, 0.1-1×, 0.1-0.5×, 0.1-0.3×, 1-100×, 1-50×, 1-30×, 1-20×, 1-10×, 1-5×, 1-3×, or 1-2× the longest dimension of the tail group of the surfactant, or the longest dimension of the head group of the surfactant. In certain embodiments, the length of the cross-link is 0.1-100 nm, 0.1-50 nm, 0.1-30 nm, 0.1-20 nm, 0.1-10 nm, 0.1-5 nm, 0.1-3 nm, 0.1-1 nm, 0.1-0.5 nm, 1-100 nm, 1-50 nm, 1-30 nm, 1-20 nm, 1-10 nm, 1-5 nm, or 1-3 nm in length. The surfactant molecules may be cross-linked head-to-head, tail-to-tail, or head-to-tail. The average number of linkage moieties on the surfactant molecules may be 1-20, 1-15, 1-10, 2-20, 2-15, 2-10, 2-8, 2-6, 3-20, 3-15, 3-10, 3-8, 3-5, 4-20, 4-15, 4-10, 4-8, 5-20, 5-15, 5-10, 5-9, 6-20, 6-15, or 6-10 linkage moieties, for example 2-20, such as 2-10, in some cases 3-10. In certain embodiments the continuous phase comprises an oil, such as a fluorinated oil as described herein. In certain embodiments the dispersed phase comprises an aqueous phase. In certain embodiments, surfactant comprises fluorinated surfactant, as described herein. In certain embodiments, dispersed phase comprises one or more components that initiate and/or promote formation of cross-links comprising the linkage moieties when in contact with the linkage moieties. In certain embodiments, the one or more components comprise one or more intermediate linkage moieties that form one or more bonds with the surfactant linkage moieties, such as when linkage moieties comprise biotin and the intermediate moiety comprises a biotin-binding moiety, such as streptavidin or streptavidin derivative. In certain embodiments, surfactant comprises fluorinated surfactant, as described herein. The cross-links may be covalent, non-covalent, or a combination thereof. Cross-links can be head-to-head, tail-to-tail, or head-to-tail. the partitions are exposed to an external stimulus that initiates and/or promotes formation of cross-links comprising the linkage moieties during and/or after partition formation, such as light. In certain embodiments, surfactant molecules comprise one or more attached biotin moieties, and cross-linking is via a biotin-binding intermediate moiety, e.g., streptavidin or a streptavidin derivative. In certain embodiments, the cross-linked network of surfactant molecules produced by the method increases the stability of the partitions in the emulsion, compared to the same emulsion without a cross-linked surfactant network, as measured by a decrease in dye diffusion of at least 20% in a dye diffusion test, such as a dye diffusion test as described herein. In certain embodiments, the cross-linked network of surfactant molecules produced by the method increases the stability of the partitions in the emulsion, compared to the same emulsion without a cross-linked surfactant network, as measured by a PCR test, such as a PCR test as described herein, by at least 20%. In certain embodiments, the cross-linked network of surfactant molecules produced by the method increases the stability of the partitions in the emulsion, compared to the same emulsion without a cross-linked surfactant network, as measured by a coalescence assay, such as a coalescence assay described herein, by at least 20%.

In certain embodiments provided herein is a method of preparing an emulsion comprising a plurality of partitions of dispersed aqueous phase in an oil continuous phase, wherein the partitions further comprise a cross-linked network of surfactant molecules at the surface of the partitions, comprising preparing an aqueous phase to be dispersed, preparing an oil phase comprising modified surfactant, wherein the modified surfactant comprises a tail portion and a head portion and further comprises linkage moieties; and mixing the aqueous phase and the oil phase to form an emulsion of a plurality of partitions of the aqueous phase in the oil, wherein the modified surfactant molecules form cross-links with each other to form a cross-linked network of surfactant molecules at the interface of the partitions with the continuous phase. The mixing can be done in bulk by vortexing, pipetting, syringing, shaking or the like, or in a microfluidic droplet forming device such as a T-junction droplet generating device, which can be a collision style device; in certain embodiments the mixing is by a microfluidic T-junction, flow focusing junction, reverse-y junction, millipede junction or a combination thereof. In certain embodiments a system for producing the emulsion is embedded within a larger instrument, such as an instrument containing a sample delivery module, a droplet generator module, a thermal cycler module, a detection module, a waste management module, or a combination thereof. The larger instrument can have microfluidic devices, tubing, containers or vats embedded. the instrument can comprise associated software that controls the instrument including but not limited to the performance of the instrument as a whole or the microfluidic device.

Also provided herein are kits. A kit can comprise a first container containing a plurality of first surfactant molecules for use in forming an emulsion, where the first surfactant molecules comprise a plurality of first linkage moieties for cross-linking to other surfactant molecules, either directly or indirectly, and packaging that contains the first container. A kit can also comprise a second container containing one or more components that initiate or promote a cross-linking reaction between the linkage moieties of the surfactants of the first container, and packaging that contains the second container. A kit can contain a third container containing a plurality of second surfactant molecules comprising a second linkage moiety, and packaging for the container. A kit can also comprise instructions for use.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising a plurality of first surfactant molecules comprising a tail portion and a head portion, wherein the first surfactant molecules comprise an average of 2-10 of a first linkage moiety per surfactant molecule wherein the first linkage moieties are configured to either
(i) link with each other under suitable conditions,
(ii) to link with a second linkage moiety under suitable conditions, wherein the second linkage moiety is attached to a second surfactant molecule comprising a tail portion and a head portion, and wherein the second linkage moiety is different from the first linkage moiety, or
(iii) to link with an intermediate linkage moiety under suitable conditions, or a combination thereof.

2. The composition of claim 1 wherein the first linkage moieties are attached to the tail portion of the first surfactant molecules.

3. The composition of claim 1 wherein the first linkage moieties are attached to the head portion of the first surfactant molecules.

4. The composition of any of the previous claims wherein the second linkage moieties are attached to the head portion of the second surfactant molecules.

5. The composition of any of claims 1-3 wherein the second linkage moieties are attached to the tail portion of the second surfactant molecules.

6. The composition of any of the previous claims wherein the linkage moieties are configured to form one or more covalent bonds under suitable conditions.

7. The composition of any of the previous claims wherein the linkage moieties are configured to form one or more noncovalent bonds under suitable conditions.

8. The composition of any of the previous claims further comprising a plurality of the second surfactant molecules, wherein the second surfactant molecules comprise an average of 2-10 of the second linkage moiety per surfactant molecule.

9. The composition of any of the previous claims wherein the first and second linkage moieties are oppositely charged.

10. The composition of any of the previous claims further comprising a continuous phase containing the first surfactant molecules.

11. The composition any of the previous claims further comprising a continuous phase containing the second surfactant molecules.

12. The composition of claim 10 or 11 further comprising a dispersed phase

13. The composition of claim 12 wherein the dispersed phase does not contain the first surfactant molecule

14. The composition of claim 12 or 13 wherein the dispersed phase does not contain the second surfactant molecule.

15. The composition of claim 12 wherein the dispersed phase contains the intermediate linkage moiety.

16. The composition of any of the previous claims further comprising a dispersed phase containing the first surfactant molecules.

17. The composition of any of the previous claims further comprising a dispersed phase containing the second surfactant molecules.

18. The composition of claim 16 or 17 further comprising a continuous phase

19. The composition of claim 18 wherein the continuous phase does not contain the first surfactant molecule

20. The composition of claim 18 wherein the continuous phase does not contain the second surfactant molecule.

21. The composition of claim 18 wherein the continuous phase contains the intermediate linkage moiety.

22. The composition of any of the previous claims wherein the surfactant moieties comprise the first linkage moiety comprising biotin and the intermediate linkage moieties comprising one or more biotin-binding moieties.

23. The composition of claim 22 wherein the biotin-binding moieties comprise streptavidin or a streptavidin derivative.

24. The composition of claim any of claims 6-23 wherein the surfactant molecules form micelles in the continuous phase.

25. The composition of any of claims 6-24 wherein the continuous phase comprises an oil.

26. The composition of claim 25 wherein the oil comprises a hydrocarbon or a silicon oil.

27. The composition of claim 25 wherein the oil comprises a fluorinated oil.

28. The composition of claim 1 wherein the surfactant is a fluorosurfactant.

29. A composition comprising an emulsion comprising partitions of a dispersed phase in a continuous phase, wherein the partitions of the dispersed phase comprise a plurality of surfactant molecules comprising a tail portion and a head portion that are situated at the interface of the partitions with the continuous phase to form a layer of surfactant molecules, and wherein the plurality of surfactant molecules are cross-linked to each other to form a cross-linked network of surfactant molecules.

30. The composition of claim 29 wherein the degree of cross-linking of the cross-linked network of surfactant molecules is 20-100%.

31. The composition of claim 29 or 30 wherein the cross-linking is via cross links having an average length of 5-500% of the length of the tail portion of the surfactant.

32. The composition of any of claims 29-31 wherein the surfactant molecules are cross-linked to each other at the tail portions of the surfactant molecules.

33. The composition of any of claims 29-31 wherein the surfactant molecules are cross-linked to each other at the head portions of the surfactant molecules.

34. The composition of any of claims 29-33 wherein a first portion of the surfactant molecules comprise a first linkage moiety that forms part of the cross-links, and wherein the average number of first linkage moieties per surfactant molecule is 2-10.

35. The composition of any of claims 29-34 wherein a second portion of the surfactant molecules comprise a second linkage moiety, different from the first linkage moiety, that forms part of the cross-links, and wherein the average number of second linkage moieties per surfactant molecule is 2-10.

36. The composition of any of claims 29-35 wherein the surfactant molecules are cross-linked via cross-links where the average length of the cross-links is 1-100 nm.

37. The composition of any of claims 29-36 wherein the dispersed phase comprises an aqueous phase and the continuous phase comprises an oil.

38. The composition of claim 37 wherein the oil comprises a fluorinated oil.

39. The composition of any of claims 29-38 wherein the surfactant comprises a fluorosurfactant.

40. The composition of any of claims 29-39 wherein the surfactant molecules are cross-linked by noncovalent bonds.

41. The composition of any of claims 29-39 wherein the surfactant molecules are cross-linked by covalent bonds.

42. The composition of any of claims 29-40 wherein the surfactant molecules comprise biotin moieties and that are cross-linked by biotin-binding moieties.

43. The composition of claim 42 wherein the biotin-binding moieties comprise streptavidin or a streptavidin derivative.

44. The composition of claim 43 wherein the surfactant molecules comprise an average of 2-10 biotin moieties per surfactant molecule.

45. The composition of any of claims 29-44 wherein the cross-linked network of surfactant molecules increases the stability of the partitions in the emulsion, compared to the same emulsion without a cross-linked surfactant network, as measured by a decrease in dye diffusion of at least 20% in a dye diffusion test.

46. The composition of any of claims 29-45 wherein the cross-linked network of surfactant molecules increases the stability of the partitions in the emulsion, compared to the same emulsion without a cross-linked surfactant network, as measured by a PCR test, by at least 20%.

47. The composition of any of claims 29-46 wherein the cross-linked network of surfactant molecules increases the stability of the partitions in the emulsion, compared to the same emulsion without a cross-linked surfactant network, as measured by a coalescence assay by at least 20%.

48. A method of conducting a process in an emulsion of partitions of a dispersed phase in a continuous phase, comprising
(i) providing the emulsion of partitions of dispersed phase in continuous phase, wherein the partitions of the dispersed phase comprise a plurality of surfactant molecules comprising a tail portion and a head portion that are situated at an interface of the partitions with the continuous phase to form a layer of surfactant molecules, and wherein the plurality of surfactant molecules are cross-linked to each other to form a cross-linked network of surfactant molecules; and
(ii) performing the process on the partitions.

49. The method of claim 48 wherein the degree of cross-linking of the cross-linked network of surfactant molecules is 20-100%.

50. The method of claim 48 or 49 wherein the cross-linking is via cross links having an average length of 5-500% of the length of the tail portion of the surfactant.

51. The method of any of claims 48-50 wherein the surfactant molecules are cross-linked to each other at the tail portions of the surfactant molecules.

52. The method of any of claims 48-50 wherein the surfactant molecules are cross-linked to each other at the head portions of the surfactant molecules.

53. The method of any of claims 48-52 wherein a first portion of the surfactant molecules comprise a first linkage moiety that forms part of the cross-links, and wherein the average number of first linkage moieties per surfactant molecule is 2-10.

54. The method of any of claims 48-53 wherein a second portion of the surfactant molecules comprise a second linkage moiety, different from the first linkage moiety, that forms part of the cross-links, and wherein the average number of second linkage moieties per surfactant molecule is 2-10.

55. The method of any of claims 48-54 wherein the surfactant molecules are cross-linked via cross-links, and where the average length of the cross-links is 1-100 nm.

56. The method of any of claims 48-55 wherein the dispersed phase is an aqueous phase and the continuous phase is a oil.

57. The method of claim 56 wherein the oil is a fluorinated oil.

58. The method of any of claims 48-57 wherein the surfactant comprises a fluorosurfactant.

59. The method of any of claims 48-58 wherein the surfactant molecules are cross-linked by noncovalent bonds.

60. The method of any of claims 48-58 wherein the surfactant molecules are cross-linked by covalent bonds.

61. The method of claim 59 wherein the surfactant molecules are cross-linked by biotin-biotin binding moiety interactions.

62. The method of claim 61 wherein the biotin-binding moieties comprise streptavidin or a streptavidin derivative.

63. The method of claim 61 wherein the surfactant molecules comprise an average of 2-10 biotin moieties per surfactant molecule.

64. The method of any of claims 48-63 further comprising forming the cross-linked network of surfactant molecules in the partitions.

65. The method of any of claims 48-64 wherein the cross-linked network of surfactant molecules has been formed by contacting continuous phase comprising a plurality of surfactant molecules that comprise at least one linkage moiety with a dispersed phase under conditions wherein the dispersed phase forms a plurality of partitions in the continuous phase, and providing conditions during and/or after the formation of the partitions that initiate and/or promote formation of cross-links comprising the linkage moieties, wherein the linkage moieties form cross-links from one surfactant molecule to at least one other surfactant molecule, to form a cross-linked network of surfactant molecules.

66. The method of claim 65 wherein dispersed phase comprises one or more components that initiate and/or promote formation of cross-links comprising the linkage moieties when in contact with the linkage moieties.

67. The method of claim 65 wherein the partitions are exposed to an external stimulus that initiates and/or promotes formation of cross-links comprising the linkage moieties.

68. The method of any of claims 48-67 wherein the process comprises chemical analysis; protein or strain engineering; nucleic acid, protein, or cell-based assays; sorting; separations; or chemical and/or biochemical synthesis; or a combination thereof.

69. The method of claim 68 wherein the process comprises a nucleic acid assay.

70. The method of claim 69 wherein the process comprises polymerase chain reaction (PCR).

71. The method of any of claims 48-70 wherein the cross-linked network of surfactant molecules increases the stability of the partitions in the emulsion, compared to the same emulsion without a cross-linked surfactant network, as measured by a decrease in dye diffusion of at least 20% in a dye diffusion test.

72. The method of any of claims 48-71 wherein the cross-linked network of surfactant molecules increases the stability of the partitions in the emulsion, compared to the same emulsion without a cross-linked surfactant network, as measured by a PCR test, of at least 20%.

73. A method for producing an emulsion of partitions of dispersed phase in continuous phase, wherein the partitions of the dispersed phase comprise a plurality of surfactant molecules comprising a tail portion and a head portion that are situated at an interface of the partitions with the continuous phase to form a layer of surfactant molecules, comprising
(i) contacting continuous phase with a dispersed phase, wherein either
(a) the continuous phase comprises a plurality of surfactant molecules that comprise at least one linkage moiety, or
(b) the dispersed phase comprises a plurality of surfactant molecules that comprise at least one linkage moiety, or
(c) both (a) and (b)
under conditions wherein the dispersed phase forms a plurality of partitions in the continuous phase; and
(ii) providing conditions during and/or after the formation of the partitions that initiate and/or promote formation of cross-links between surfactant molecules comprising the linkage moieties, to form a cross-linked network of surfactant molecules.

74. The method of claim 73 wherein the continuous phase comprises surfactant molecules comprising linkage moieties and the dispersed phase does not.

75. The method of claim 73 or 74 wherein the linkage moieties and other components of the cross-links, if present, form cross-links that have a length that is 5-500% of the length of the tail portion of the surfactant.

76. The method of any of claims 73-75 wherein the surfactant molecules comprise an average of 2-10 linkage moieties per surfactant molecule.

77. The method of any of claims 73-76 wherein the conditions and/or number of linkage moieties are such that the degree of completion of the cross-linked network of surfactant molecules is 20-100%.

78. The method of any of claims 73-77 wherein the linkage moieties are attached to the tail portions of the surfactant molecules and the cross-links form between the tail portions of the surfactant molecules.

79. The method of any of claims 73-77 wherein the linkage moieties are attached to the head portions of the surfactant molecules and the cross-links form between the head portions of the surfactant molecules.

80. The method of any of claims 73-79 wherein dispersed phase comprises one or more components that initiate and/or promote formation of cross-links comprising the linkage moieties when in contact with the linkage moieties.

81. The method of claim 60 wherein the one or more components comprise one or more intermediate linkage moieties that form one or more bonds with the surfactant linkage moieties.

82. The method of claim 81 wherein the surfactant linkage moieties comprise biotin and the intermediate linkage moieties comprise biotin-binding moieties.

83. The method of claim 82 wherein the biotin-binding moieties comprise streptavidin and/or streptavidin derivatives.

84. The method of any of claims 73-83 wherein the partitions are exposed to an external stimulus that initiates and/or promotes formation of cross-links comprising the linkage moieties.

85. The method of claim 84 wherein the external stimulus comprises light.

86. The method of any of claims 73-85 wherein the continuous phase comprise an oil and the dispersed phase comprises an aqueous phase.

87. The method of claim 86 wherein the oil comprises a fluorinated oil.

88. The method of any of claims 73-87 wherein the surfactant comprises a fluorosurfactant.

89. The method of any of claims 73-88 wherein the cross-linked network of surfactant molecules increases the stability of the partitions in the emulsion, compared to the same emulsion without a cross-linked surfactant network, as measured by a decrease in dye diffusion of at least 20% in a dye diffusion test.

90. The method of any of claims 73-89 wherein the cross-linked network of surfactant molecules increases the stability of the partitions in the emulsion, compared to the same emulsion without a cross-linked surfactant network, as measured by a PCR test of at least 20%.

91. A method of preparing an emulsion comprising a plurality of partitions of dispersed aqueous phase in an oil continuous phase, wherein the partitions further comprise a cross-linked network of surfactant molecules at the surface of the partitions, comprising preparing an aqueous phase to be dispersed, preparing an oil phase comprising modified surfactant, wherein the modified surfactant comprises a tail portion and a head portion and further comprises linkage moieties; and mixing the aqueous phase and the oil phase to form an emulsion of a plurality of partitions of the aqueous phase in the oil, wherein the modified surfactant molecules form cross-links with each other to form a cross-linked network of surfactant molecules at the interface of the partitions with the continuous phase.

92. The method of claim 91 wherein the mixing is done in bulk by vortexing, pipetting, syringing, shaking.

93. The method of claim 91 wherein the mixing is by a microfluidic droplet forming device.

94. The method of claim 93 wherein the mixing is by a microfluidic T-junction, flow focusing junction, reverse-y junction, millipede junction or a combination thereof.

95. The method of any of claims 91-94 wherein a system for producing the emulsion is embedded within a larger instrument.

96. The method of claim 95 wherein the instrument is an instrument containing a sample delivery module, a droplet generator module, a thermal cycler module, a detection module, a waste management module, or a combination thereof.

97. The method of claim 95 wherein the larger instrument has microfluidic devices, tubing, containers or vats embedded.

98. The method of claim 95 wherein the instrument comprises associated software that controls the instrument including but not limited to the performance of the instrument as a whole or the microfluidic device.

99. A method for preventing coalescence of partitions in an emulsion comprising partitions of dispersed phase in a continuous phase, comprising forming a cross-linked network of surfactant molecules at interfaces between the partitions and the continuous phase.

100. The method of claim 99 wherein the partitions have an average diameter of greater than 1, 5, 10, 20, 30, 40, 50, 60, 70, or 80 um, such as greater than 10 um, in diameter.

101. The method of claim 98 or 99 wherein the emulsion is at a temperature greater than 30, 40, 45, 50, 55, 60, 70, 80, or 90° C., such as greater than 70° C.

102. A method for transporting, thermal cycling, incubating, sorting, or analyzing an emulsion comprising partitions of dispersed phase in a continuous phase in a microfluidic device, wherein the droplets comprising a network of cross-linked surfactant molecules at the interface of the partitions and the continuous phase.

103. A method of performing PCR in partitions of an emulsion comprising partitions of aqueous phase in a continuous phase, wherein the partitions comprise cross-linker, polymerase, nucleotides, template DNA, primers, and probes/or DNA binding dyes, and the continuous phase comprises surfactant molecules comprising one or more linkage moieties.

104. A method comprising
(i) forming an emulsion comprising a plurality of partitions comprising dispersed phase, in a continuous phase, wherein the partitions comprise a surfactant layer comprising a plurality of surfactant molecules at an interface with the continuous phase;
(ii) cross-linking surfactant molecules at the interface to form a cross-linked surfactant network;
(iii) performing a process on the partitions; and
(iv) treating the cross-linked surfactant network to decrease a degree of cross-linking.

105. The method of claim 104 further comprising breaking open a plurality of the partitions to release dispersed phase in the partitions.

106. The method of claim 104 or 105 wherein the surfactants comprise fluorosurfactants and the continuous phase comprises a fluorinated oil.

107. The method of any of claims 104-106 wherein the process comprises a polymerase chain reaction.

108. A composition comprising a continuous phase wherein the continuous phase comprises a plurality of surfactant molecules that comprise linkage moieties attached to the surfactant molecules, wherein the moieties form cross-links with each other under suitable conditions.

109. The composition of claim 108 wherein the continuous phase is to be used in the preparation of an emulsion

110. The composition of claim 108 or 109 wherein the moieties do not substantially interact to cross-link in the continuous phase.

111. The composition of any of claims 108-110 wherein the moieties form non-covalent cross-links under suitable conditions.

112. The composition of any of claims 108-111 wherein the moieties form covalent cross-links under suitable conditions.

113. The composition of any of claims 108-112 wherein the continuous phase comprises an oil.

114. The composition of claim 113 wherein the oil is a fluorinated oil.

115. The composition of claim 114 wherein the fluorinated oil is (3-ethoxy-1,1,1,2,3,4,4,5,5,6,6,6-dodecafluoro-2-trifluoromethyle-hexane), methyl nonafluorobutyl ether, methyl nonafluoroisobutyl ether, ethyl nonafluoroisobutyl ether, ethyl nonofluorobutul ether, (pentane, 1,1,1,2,2,3,4,5,5,5-decafluoro-3-methoxy-4-(trifluoromethyl-)), isopropyl alcohol, (1,2-trans-dicholorethylene), (butane,1,1,1,2,2,3,3,4,4-nonafluoro-4-methoxy-), (1,1,1,2,2,4,5,5,5-nonafluoro-4-(trifluoromethyl)-3-pentanone), (furan,2,3,3,4,4-pentafluorotetrahydro-5-methoxy-2,5-bis[1,2,2,2-tetrafluoro-1-(trifluoromethyl)ethyl]-), perfluoro compounds comprising between 5 and 18 carbon atoms, polychlorotrifluoroethylene, (2,2,2-trifluoroethanol), Novec 8200™, Novec 71DE™, Novec 7100™, Novec 7200DL™, Novec 7300DL™, Novec 71 IPA™, Novec 72FL™, Novec 7500™, Novec 71DA™, Novec 7100DL™, Novec 7000™, Novec 7200™, Novec 7300™, Novec 72DA™, Novec 72DE™, Novec 649™, Novec 73DE™, Novec 7700™, Novec 612™, FC-40™, FC-43™, FC-70 ™, FC-72™, FC-770 ™, FC-3283™, FC-3284™, PF-5056™, PF-5058™, Halocarbon 0.8™, Halocarbon 1.8™, Halocarbon 4.2™, Halocarbon 6.3™, Halocarbon 27™, Halocarbon 56™, Halocarbon 95™, Halocarbon 200™, Halocarbon 400™, Halocarbon 700™, Halocarbon 1000N™, Uniflor 4622R™, Uniflor 8172™, Uniflor 8472CP™, Uniflor 8512S™, Uniflor 8731™, Uniflor 8917™, Uniflor 8951™, TRIFLUNOX 3005™, TRIFLUNOX 3007™, TRIFLUNOX 3015™, TRIFLUNOX 3032™, TRIFLUNOX 3068™, TRIFLUNOX 3150™, TRIFLUNOX 3220™, or TRIFLUNOX 3460™ or a combination thereof.

116. The composition of claim 114 wherein the fluorinated oil comprises (3-ethoxy-1,1,1,2,3,4,4,5,5,6,6,6-dodecafluoro-2-trifluoromethyle-hexane); (furan,2,3,3,4,4-pentafluorotetrahydro-5-methoxy-2,5-bis[1,2,2,2-tetrafluoro-1-(trifluoromethyl)ethyl]-); perfluoro compounds comprising between 5 and 18 carbon atoms; or a combination thereof.

117. The composition of any of claims 108-116 wherein the surfactants comprise fluorosurfactants.

118. The composition of claim 117 wherein the fluorosurfactant comprises fluorosurfactants having head and tail moieties linked by ether, amide, or carbamide bonds; fluorosurfactants having a polyethylene moiety linked to a fluorocarbon moiety through a carbamide, ether, or amide bond, or a combination thereof.

119. The composition of claim 117 wherein the fluorosurfactant comprises a polyethylene moiety linked to a fluorocarbon moiety with a carbamide, amide, or ether bond.

120. The composition of any of claims 108-119 wherein the cross-links formed under suitable conditions comprise direct cross-links.

121. The composition of any of claims 108-119 wherein the cross-links formed under suitable conditions comprise indirect cross-links.

122. The composition of claim 121 wherein the linkage moieties comprise biotin.

123. The composition of any of claims 108-122 wherein each surfactant molecule comprises an average of at least 2 linkage moieties.

124. The composition of any of claims 108-122 wherein each surfactant molecule comprises an average of at least 4 linkage moieties.

125. The composition of any of claims 108-124 wherein the linkage moieties of the surfactant molecules are such that when they react under suitable conditions, the resulting cross-link between surfactant molecules is 1-100 nm.

126. The composition of any of claims 108-125 wherein the cross-link that forms under suitable conditions between linkage moieties has a bond strength of between 20 and 200 kJ/mole.

127. The composition of any of claims any of claims 108-126 wherein the concentration of surfactant molecules in the continuous phase is 0.5% to 2% w/v.

128. A composition comprising a dispersed phase for use in formation of an emulsion of partitions of the dispersed phase in a continuous phase wherein the partition interface with the continuous phase in the emulsion comprises a plurality of surfactant molecules comprising cross-linking moieties, wherein the dispersed phase comprises one or more components that initiate and/or promote a cross-linking process between the cross-linking moieties under suitable conditions.

129. The composition of claim 128 wherein the dispersed phase is not in contact with a continuous phase.

130. The composition of claim 128 or 129 wherein the dispersed phase comprises an aqueous phase.

131. The composition of any of claims 128-130 wherein the dispersed phase further comprises one or more additional components that initiate and/or promote a non-cross-linking process under suitable conditions.

132. The composition of claim 131 wherein the non-cross-linking process comprises a chemical reaction.

133. The composition of claim 132 wherein the components comprise nucleic acids and components for conducting polymerase chain reaction.

134. The composition of any of claims 128-133 wherein the cross-linking process comprises a covalent interaction.

135. The composition of any of claim 128-134 wherein the cross-linking process comprises a non-covalent process.

136. The composition of any of claims 128-135 wherein the cross-linking process comprises linking linkage moieties of the surfactant molecules through one or more intermediate linker moieties, and wherein the dispersed phase further comprises a plurality of the intermediate linker moieties.

137. The composition of claim 136 wherein the intermediate linker moiety comprises a biotin-binding moiety.

138. The composition of claim 137 wherein the biotin-binding moiety comprises avidin, streptavidin, a streptavidin derivative, or a combination thereof.

139. The composition of any of claims 128-138 wherein the concentration of the one or more components is 10 nanomolar to 10 millimolar for each component.

140. A emulsion composition comprising a plurality of partitions of a dispersed phase in a continuous phase, wherein
(i) interfaces between the dispersed phase partitions and the continuous phase comprises a plurality of surfactant molecules;
(ii) the surfactant molecules of the interfaces are cross-linked by one or more linkage moieties between surfactant molecules;
(iii) at least a portion of the partitions contain first components that, under suitable conditions, undergo a process to produce second components; and
(iv) the continuous phase comprises reporter moieties that interact with the second components to produce a signal indicating the presence and/or abundance of the second components.

141. The composition of claim 140 wherein the first and second components are the same or nearly the same.

142. The composition of claim 140 or 141 wherein the first component is a nucleic acid of interest and the second component is a product of amplification of the nucleic acid.

143. The composition of any of claims 140-142 wherein the linkage moieties have one or more properties that promote entrance of the reporter molecule into the partitions.

144. A method of performing an emulsion flow process comprising
(i) providing a continuous phase comprising
(a) a continuous phase, and
(b) surfactant molecules with linkage moieties
(ii) providing a dispersed phase, separate from the continuous phase, that does not comprise surfactant molecules with linkage moieties, and that comprises one or more components that initiate and/or promote formation of cross-links between surfactant molecules, wherein the cross-links comprise the linkage moieties;
(iii) flowing the continuous phase and the dispersed phase into a partition generator that generates an emulsion of a plurality of partitions comprising the dispersed phase in the continuous phase, and
(iv) during and/or after partition formation, forming cross-links between surfactant molecules that comprise the linkage moiety to form a cross-linked network of surfactant molecules at the interface of the partitions with continuous phase; and
(iii) flowing the emulsion through a process system that performs one or more operations on the partitions of the emulsion.

145. A composition comprising a plurality of first surfactant molecules comprising a tail portion and a head portion, wherein the first surfactant molecule comprises a first linkage moiety attached to the first surfactant molecule, wherein the first linkage moiety is configured to participate in formation of cross-links between the first surfactant molecule and a second surfactant comprising a second linkage moiety under suitable conditions.

146. The composition of claim 145 wherein the first and second linkage moieties have the same structure.

147. The composition of claim 145 or 146 wherein the first surfactant molecule comprises a plurality of linkage moieties.

148. The composition of claim 147 wherein the first surfactant molecule is attached to an average of 2-10 linkage moieties.

149. The composition of any of claims 145-148 wherein the first linkage moiety is attached to the tail portion of the surfactant molecule.

150. The composition of any of claims 145-148 wherein the first linkage moiety is attached to the head portion of the surfactant molecule.

151. The composition of claim any of claims 145-150 wherein the length of the first linkage moiety is 5-500% of the length of the head portion of the surfactant molecule.

152. The composition of any of claims 145-151 wherein the first surfactant molecule is a nonionic, anionic, cationic, or zwitterionic surfactant.

153. The composition any of claims 145-151 wherein the first surfactant molecule is a fluorosurfactant.

154. The composition of any of claims 145-153 wherein the linkage moiety is covalently attached to the surfactant molecule.

155. The composition of any of claims 145-154 wherein the linkage moiety is configured to bind to an intermediate moiety under suitable conditions and not to another linkage moiety.

156. The composition of claim 155 wherein the linkage moiety comprises biotin.

157. The composition of claim 155 wherein the intermediate moiety comprises a biotin-binding moiety.

158. The composition of any of claims 145-157 wherein the plurality of first surfactant molecules is contained in a continuous phase.

159. A method of modifying a first surfactant molecule comprising a tail portion and a head portion, comprising attaching a plurality of first linkage moieties to the first surfactant molecule, wherein the first linkage moiety is configured to participate in formation of cross-links between the first surfactant molecule and a second surfactant molecule comprising a second linkage moiety under suitable conditions.

160. The method of claim 159 comprising attaching an average of 2-10 of the first linkage moiety to the first surfactant molecule.

161. The method of claim 159 or 160 comprising attaching the plurality of first linkage moieties to the tail portion of the surfactant molecule.

162. The method of claim 159 or 160 comprising attaching the plurality of first linkage moieties to the head portion of the surfactant molecule.

163. The method of any of claims 159-162 wherein the length of the first linkage moiety is 5-500% of the length of the head portion of the surfactant molecule.

164. The method of any of claims 159-163 wherein the first surfactant molecule is a nonionic, anionic, cationic, or zwitterionic surfactant.

165. The method of any of claims 159-163 wherein the first surfactant molecule is a fluorosurfactant.

166. The method of any of claims 159-165 comprising covalently attaching the linkage moiety to the first surfactant molecule.

167. The method of any of claims 159-166 wherein the linkage moiety is configured to bind to an intermediate moiety under suitable conditions and not to another linkage moiety.

168. The method of claim 167 wherein the linkage moiety comprises biotin.

169. The method of claim 168 wherein the intermediate moiety comprises a biotin-binding moiety.

170. A kit comprising
(i) a container comprising a plurality of surfactant molecules for use in forming an emulsion, wherein the surfactant molecules comprise a plurality of linkage moieties for cross-linking to other surfactant molecules;
(ii) packaging that contains the container of (i).
